(19) United States
(12) Patent Application Publication
(10) Pub. No.: US 2023/0323433 A1
(43) Pub. Date: Oct. 12, 2023

(54) Methods of Assaying a Biological Cell

(71) Applicant: Berkeley Lights, Inc., Emeryville, CA (US)

(72) Inventors: Matthew Asuka Kubit, Berkeley, CA (US); Joshua David Mast, Hayward, CA (US); John Junyeon Kim, Berkeley, CA (US); Alexander Gerald Olson, Hayward, CA (US); Preston Lock Ng, Mountain View, CA (US); Arlvin Louis Ellefson, San Francisco, CA (US); Shruthi Sreedhar Kubatur, Alameda, CA (US); Vincent Haw Tien Pai, Berkeley, CA (US); Minha Park, Brisbane, CA (US); Po-Yuan Tung, South San Francisco, CA (US); Jason C. Briggs, Pleasanton, CA (US); Patrick N. Ingram, Emeryville, CA (US); Katrine Elise Dailey, Albany, CA (US); Maryam Shansab, Oakland, CA (US); Jason M. McEwen, El Cerrito, CA (US); Adrienne T. Higa, San Francisco, CA (US); Hongye Zhou, Alameda, CA (US); Zhen Hu, Redwood City, CA (US); John A. Tenney, Piedmont, CA (US)

(73) Assignee: Berkeley Lights, Inc., Emeryville, CA (US)

(21) Appl. No.: 18/178,927

(22) Filed: Mar. 6, 2023

Related U.S. Application Data
(63) Continuation of application No. PCT/US2021/048976, filed on Sep. 3, 2021.
(60) Provisional application No. 63/211,337, filed on Jun. 16, 2021.
(60) Provisional application No. 63/080,960, filed on Sep. 21, 2020.
(60) Provisional application No. 63/075,269, filed on Sep. 7, 2020.

(51) Int. Cl. C12Q 1/6834; C12Q 1/6844; C12Q 1/6804

(52) U.S. Cl. CPC C12Q 1/6834; C12Q 1/6804; C12Q 1/6844; C12Q 2600/16

(57) ABSTRACT

Disclosed herein are methods for performing assays, including general functional assays, on a biological cell. Also disclosed herein are methods of barcoding the 5′ ends of RNA from a biological cell and methods of preparation of expression constructs from the barcoded RNA. The barcoded RNA can encode proteins of interest, such as B cell receptor (BCR) heavy and light chain sequences. The expression constructs can be generated individually or in a paired/multiplexed manner, allowing rapid re-expression of individual proteins or protein complexes.

This application is a continuation of International Application No. PCT/US2021/048976, filed Sep. 3, 2021, which claims the benefit under 35 U.S.C. 119(e) of U.S. Provisional Application No. 63/080,960, filed on Sep. 21, 2020; U.S. Provisional Application No. 63/075,269, filed on Sep. 7, 2020; and U.S. Provisional Application No. 63/211,337, filed on Jun. 16, 2021, each of which disclosures is herein incorporated by reference in its entirety.

The present application contains a Sequence Listing which has been submitted electronically in XML format. Said XML copy, created on Feb. 27, 2023, is named “2023-02-27_01149-0018-00US_ST26.xml” and is 104,832 bytes in size. The information in the electronic format of the sequence listing is incorporated herein by reference in its entirety.

### INTRODUCTION AND SUMMARY

This application relates to methods of assaying a biological cell. This application also relates to methods of barcoding the 5′ ends of RNA from a biological cell and methods of preparation of expression constructs from the barcoded RNA.

Over the past three decades, antibody therapies have been developed for a host of different diseases, ranging from autoimmune disorders to infectious diseases and cancer. Cell-based assays enable screening against native antigens and, therefore, may accelerate therapeutic antibody lead candidate selection. However, the time it takes to screen cells for lead candidates using a typical workflow significantly adds to the drug development timeline. For example, after immunizing the animal and harvesting the antibody-producing B lymphocytes (or B cells) from the spleen, bone marrow, or lymph node, it can take at least 12 weeks to produce a hybridoma and screen through all of the potential hits, prolonging the development process.

Recent development of on-chip screening systems allows more rapid selection of lead candidates. For example, several tens of thousands of cells can be cloned in parallel in chambers of the microfluidic device, and multiple assays can be performed for thorough characterization of promising lead candidates. Automated cell lysis and reverse transcription can be performed on chip to generate stable cDNA molecules, which can be subsequently recovered for paired heavy/light chain amplification and sequencing. Due to the short life span of antibody producing cells (especially plasma cells), however, the total number of sequences that can be recovered is limited by export capacity within that time frame. Further, validating antibody sequences obtained requires cloning of the exported cDNAs, re-expression of antibodies in culture, and off-chip assays. Accomplishing this work using traditional cloning and re-expression methods can be slow and labor intensive. Accordingly, a need exists for antibody discovery workflows that allow for rapid selection and/or re-expression of antibodies.

Disclosed herein are methods for providing one or more barcoded cDNA sequences from a biological cell. Also, disclosed herein are methods of preparing an expression construct for protein expression from the captured barcoded cDNA sequences.

In some embodiments, a method of assaying for inhibition of a specific binding interaction between a first molecule and a second molecule is provided. In some embodiments, the method is performed within a microfluidic device having a chamber, the method comprising: introducing a micro-object into the chamber of the microfluidic device, wherein the micro-object comprises a plurality of first molecules; introducing a cell into the chamber, wherein the cell is capable of producing a molecule of interest; incubating the cell in the chamber, in the presence of the micro-object, and under conditions conducive to production and secretion of the molecule of interest; after incubating the cell in the chamber, introducing the second molecule into the chamber, wherein the second molecule is bound to a detectable label; and monitoring an accumulation of the second molecule on the micro-object, wherein an absence or diminishment of accumulation of the second molecule on the micro-object indicates that the molecule of interest inhibits binding of the first molecule to the second molecule.

In some embodiments, introducing the micro-object into the chamber may further include selecting the single micro-object based on detecting a condition of viability for the micro-object. Detecting the condition of viability may further include employing a machine-learning algorithm to assign a probability of viability to the micro-object.

In some embodiments, a method of providing one or more barcoded cDNA sequences from a biological cell is provided. In some embodiments, the method includes providing the biological cell within a chamber; providing a capture object in the chamber, the capture object comprising a label, a plurality of first oligonucleotides, and a plurality of second oligonucleotides, wherein each first oligonucleotide of the plurality comprises a barcode sequence, and a sequence comprising at least three consecutive guanine nucleotides at a 3′ end, wherein each second oligonucleotide of the plurality comprises a capture sequence, lysing the biological cell and allowing RNA released from the lysed biological cell to be captured by the capture sequences of the plurality of second oligonucleotides, thereby forming captured RNA; and reverse transcribing the captured RNA, thereby producing one or more barcoded cDNA sequences, each comprising an oligonucleotide sequence complementary to a corresponding one captured RNA covalently linked to the reverse complement of the barcode sequence of the first oligonucleotide.

In some embodiments, introducing the biological cell into the chamber may further include selecting the biological cell based on detecting a condition of viability for the biological cell. Detecting the condition of viability may further include employing a machine-learning algorithm to assign a probability of viability to the biological cell.

In some embodiments, a capture object is provided, the capture object comprising a label, a plurality of first and second oligonucleotides wherein each first oligonucleotide of the plurality comprises a barcode sequence, and a sequence comprising at least three consecutive guanine nucleotides at a 3′ end and wherein each second oligonucleotide of the plurality comprises a capture sequence. In some embodiments, a kit is provided, including a plurality of capture objects described herein. In some embodiments, a kit is provided, including a microfluidic device having a plurality of chambers, and a plurality of capture objects, each having a plurality of first and second oligonucleotides, according to any of the capture objects described herein.

In some embodiments, a method is provided for introducing a micro-object into a chamber of a microfluidic device, including: introducing one or more micro-objects into a flow region of a microfluidic device; determining a condition of viability of the one or more micro-objects; selecting at least one micro-object having viability from the one or more micro-objects; and introducing the at least one micro-object into a chamber of the microfluidic device. In some embodiments, the determining the condition of viability is performed without labelling the one or more micro-objects, e.g., the micro-object are label-free. In some embodiments, determining the condition of viability may further include employing a machine-learning algorithm to assign a probability of viability to each of the one or more micro-objects. In some embodiments, the machine-learning algorithm may include a trained machine-learning algorithm, where the training may include imaging micro-objects having a label demarking a condition of viability. The micro-objects having the label form a training set of molecules, and may be micro-objects of the same kind as the one or more micro-objects introduced to the flow channel of the microfluidic device.

These and other features and advantages of the disclosed methods will be set forth or will become more fully apparent in the description that follows and in the appended claims. The features and advantages may be realized and obtained by means of the objects and combinations particularly pointed out in the appended examples, partial listing of embodiments, and claims. Furthermore, the features and advantages of the described methods may be learned by the practice or will be obvious from the description, as set forth hereinafter.

It is to be understood that both the foregoing general description and the following detailed description are exemplary and explanatory only and are not restrictive of the claims. The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 illustrates a workflow for antibody discovery according to some embodiments of the disclosure.

FIG. 7 illustrates RNA capture and reverse transcription to generate a barcoded cDNA sequence according to certain embodiments of the present disclosure.

FIG. 8 shows formation of an expression construct for an antibody heavy chain using transcriptionally-active PCR (TAP) according to certain embodiments of the present disclosure.

FIG. 9 illustrates a schematic representation of demultiplexing barcoded cDNA sequences according to certain embodiments of the present disclosure.

FIG. 10 is a schematic representation of an embodiment of a capture object of the present disclosure.

FIG. 11 is a schematic representation of a method for aligning sequence fragments to provide a V(D)J sequence of a plasma cell according to some embodiments of the disclosure.

FIG. 12A is a graphical illustration of sequence alignment in a reference-based assembly algorithm according to some embodiments of the disclosure.

FIG. 12B is a graphical illustration of sequence alignment in a reference-based assembly algorithm according to some embodiments of the disclosure.

FIG. 12C is a graphical illustration of sequence alignment in a reference-based assembly algorithm according to some embodiments of the disclosure.

FIG. 13 is a schematic representation of a method for aligning sequence fragments to provide oligonucleotide sequences of the heavy and light chains of a B cell receptor sequence.

FIGS. 14A-14B are graphical illustrations of sequence alignment in a reference-based assembly algorithm according to some embodiments of the disclosure.

FIG. 15 is a schematic representation of a Sanger sequencing-based model for sequence recognition.

FIGS. 16A-16C show multiple recombinant PD-L1 bead binding assays, performed simultaneously or in parallel. The recombinant PD-L1 bead binding assay performed in-channel (FIGS. 16A-16C, top row) down-selects for antibodies that bind to the PD-L1 coated beads. In the examples shown, both the blocking and non-blocking antibodies bind the PD-L1 coated beads. The cell binding assay performed in-pen (FIGS. 10A-10C, middle row) was performed at the same time as the recombinant PD-L1 bead binding assay and identifies antibodies that bind to native PD-L1 expressed by a reporter cell. In the examples shown, both the blocking and non-blocking antibodies bound the reporter cell. The ligand/receptor-blocking assay identifies antibodies with the ability to block the PD-1/PD-L1 interaction (FIGS. 16A-16C, bottom row). In the examples shown, the blocking antibodies are detected by non-fluorescent reporter cells, while the non-blocking antibodies result in fluorescent reporter cells.

FIG. 17 shows that deeper characterization enables down-selection of high quality lead candidates. Fewer than 2% of screened plasma B cells secreted antibodies that bound recombinant PD-L1. Of these 598 antibodies, only 273 antibodies (fewer than 1% of plasma B cells screened) bound to the cell-based PD-L1 (as shown in CHO-K1 cell binding assay). Further screening with the ligand/receptor-blocking assay down-selected 46 lead candidates (0.1% of plasma B cells screened).

FIG. 18 shows a large number of functionally-active lead candidates are identified by screening B cells from multiple organs using the methods according to certain embodiments of the present disclosure. Three times (3x) more ligand/receptor blocking antibodies were identified from plasma B cells in the bone marrow as compared to the spleen (34 of the 46 candidates, or 74%).

FIGS. 19A-19D show that re-expressed antibodies exhibited the expected functional behavior when evaluated using conventional well-plate-based assays. 20 out of 24 of the lead candidates that were cloned and re-expressed exhibited binding affinity to the PD-L1 extracellular domain (ECD) in an ELISA (FIG. 19A) and to the full-length PD-L1 protein expressed by CHO-K1 cells in a FACS assay (FIG. 19B). The same 20 antibodies also bound to the cynomolgus PD-L1 protein that would most likely be used in obligate animal studies during the pre-clinical phase of drug development (FIG. 19C). Finally, 20 of the purified antibodies effectively blocked the PD-1/PD-L1 interaction (FIG. 19D). 20% of these antibodies had IC50 values comparable to PD-1/PD-L1 blocking antibodies currently in the clinic.

FIG. 20 is a photographic representation of stained cells disposed within the microfluidic device imaged at brightfield (top), FITC (calcein) and DAPI (Zombie) (middle), and CY5 (CD138) (bottom) cube channels (filter cubes). There are cells located both in channel and in chambers, which may be difficult to determine in the brightfield image (top). As examples, circle 2010 circles three cells that are calcein-positive as shown in the middle image; circle 2020 circles another four cells, among which, three of them are Zombie-positive and one of them is calcein-positive as shown in the middle image.

FIG. 21 shows three boxplots illustrating the fluorescence levels (brightness) of cells stained with calcein (top), Zombie (middle), and CD138 (bottom) inside the microfluidic device respectively. The thresholds for each channel to determine whether cells are stained positive are based on the 2 standard deviations (stdev) above the average for each channel. n = 5837 cells.

FIG. 22 shows boxplots comparing the fluorescence levels (brightness) of cells stained with Zombie (top), calcein (middle), and CD138 (bottom) in-channel and in-pen. Data collected from three microfluidic devices (chips) are presented: D70161, n = 4403 in channel, n = 3179 in cells; D70163, n = 4698 cells in channel, n = 3561 cells in pen; D70169, n = 4523 cells in channel, 3563 cells in pen. Outliers were excluded by gating cell diameter (10 microns), and cell debris/clump verified in Image Analyzer 2.1 were also excluded. Each dot represents a plasma cell in channels. Whiskers extend to data within 1.5 times the IQR.

FIG. 23 shows a graph illustrating the subpopulation frequency differences between in-channel and in-pen cells stained with CD138 (top), Zombie (middle), and calcein (bottom) based on the threshold from unstained cells (328.9 AFU for calcein, 4101.7 AFU for Zombie, 2024.6 AFU for CD138).

FIG. 24 shows density scatter plots illustrating the relationship of CD138, calcein, Zombie expression levels of cells comparing in-channel and in-pen locations. The data are shown in log scale. From the plots showing the calcein and Zombie expression levels, two subpopulations can be clearly observed; while a major subpopulation was observed from the comparison between Zombie and CD138 expression levels. The density scatter plots demonstrate that calcein separates the live and dead subpopulations with the largest fluorescence separation.

FIGS. 25A-25B show graphs illustrating the data from an off-chip FACS analysis showing the signal intensities of lives cells (FIG. 25A) or dead cells (FIG. 25B) (the scatter plot) and the backgating analysis (the three plots on the right of each panel). The graphs verify that the on-chip data match very well with the off-chip flow cytometry data. The analysis was performed on a BD FACS Celesta Cell Analyzer, and the data was analyzed using the FlowJo v10 software.

FIGS. 26A-26B shows the scatter plots illustrating the data from an off-chip FACS analysis. Those scatter plots demonstrate the correlation between Zombie (DAPI) vs. Calcein (FITC) (FIG. 26A) and Zombie (DAPI) vs. CD138 (AF647) (FIG. 26B).

FIG. 27 demonstrates three typical morphologies of cells observed under brightfield that may be used to correlate with assigned values of viability of the cells.

FIG. 28 shows the correlation between calcein intensity and the morphologies of cells.

FIG. 29 shows a combined image taken at brightfield and FITC channel (calcein).

FIG. 30 shows an image of B cells (denoted with “+”) detected in FIG. 29, which was used as the input to the live/dead classification model.

FIG. 31 shows an expected output for the live/dead classification model. Each live cell is denoted with a solid circle; while each dead cell is denoted with a ‘+’ .

FIG. 32 shows the detection of a stain-free sample performed by a trained live/dead classification model. The image in left shows the live cells (in solid white circle) and dead cells (in solid black circle) recognized by the algorithm. The image in right is a brightfield image annotated by human eyes verifying the algorithm was accurate.

FIG. 33 shows a combined image taken at brightfield and FITC channel (calcein), which demonstrates that the live/dead classification model is properly classifying detected B cells as live/dead based on only an OEP image. Each live cell is denoted with a solid circle; while each dead cell is denoted with a ‘+’ .

FIG. 34 shows the same image as FIG. 33 but with the OEP channel turned off. Each live cell is denoted with a solid circle ; while each dead cell is denoted with a ‘+’.

FIGS. 35A-35B show two plots demonstrating how the setting of threshold is affecting the precision (FIG. 35A) and recall (FIG. 35B) rate of the live/dead detection.

FIG. 36 shows a plot illustrating the F1 score, which is the harmonic mean calculated from the precision and recall data in FIGS. 35A-35B.

FIG. 37 is a graphical illustration of the frequency of amplicons with the expected barcode from PCR reactions using barcode specific forward primers to amplify cDNA according to some embodiments of the disclosure.

FIG. 38 shows on-chip images of channels filled by Jurkat cells at a density of 1.7×10^8 (upper) and by K562 cells at a density of 1×10^8 (lower) respectively.

FIG. 39 illustrates a generalized schematic of a receptor blocking assay.

FIG. 40 illustrates a generalized schematic of a ligand blocking assay.

FIG. 41 illustrates a receptor blocking assay on chip. Secreting B cells are shown as “B” circles. Reporter cells are shown as “R” circles. Dye-labeled ligands are shown as “L” rectangles. The upper panel demonstrates the case where the secreted antibodies bind the reporter and block ligand binding. The lower panel demonstrates the case where the secreted antibodies are non-blocking, allowing ligand to bind to the reporter.

FIG. 42 illustrates a ligand blocking assay on chip. Antibody-secreting B cells are shown as “B” circles. Reporter cells are shown as “R” circles. Dye-labeled ligands are shown as “L” rectangles. The top panel demonstrates the case where the secreted antibodies bind the ligand and block binding to the reporter. The middle panel demonstrates the case where the secreted antibodies are non-blocking, allowing the ligand to bind to the reporter. The bottom panel demonstrates the case where the secreted antibodies bind and block the ligand, but because the ligand concentration significantly exceeds the secreted antibody concentration, some of the ligand may reach and bind to the reporter.

FIG. 43 illustrates the design of a receptor blocking assay. CD3 is endogenously expressed on the surface of the Jurkat reporter cell and will bind both secreted OKT3 antibody as well as the dye-labeled HIT3a (ligand). Pens with OKT3 secreting hybridoma cells should block HIT3a binding and the reporter cells will appear dark in the ligand imaging channel. Pens lacking OKT3 secreting cells will be non-blocking, and HIT3 can freely bind to the reporter cells, which will appear bright in the ligand imaging channel.

FIG. 44A shows the intensity distribution of background (MeanBackgroundBrightness) and reporter cells (MaxBrightness) as a function of ligand concentration.

FIG. 44B shows the median, 75th and 95th percentile of the background subtracted reporter cell intensity (Max - BG) as a function of ligand concentration.

FIG. 45A shows the intensity distribution of background (MeanBackgroundBrightness) and reporter cells (MaxBrightness) as a function of time.

FIG. 45B shows the median of the background subtracted reporter cell intensity (Max BG) as a function of ligand concentration.

FIGS. 46A-46B show the distribution of Mean Background Brightness ( “BG”) and MaxBrightness ( “Max”) just before (FIG. 46A) and 5 min after flushing (FIG. 46B) the chip with media. The black vertical line (“Threshold”) indicates a cell detection threshold defined by the average background signal plus 2 standard deviations.

FIG. 47A shows background subtracted reporter cell intensity histograms just before and 5 min after flushing with media .

FIG. 47B shows background (MeanBackgroundBrightness) and the fraction of reporter cells above detection threshold () as a function of time.

FIG. 48 is a heatmap showing that pen-based false positive hit rates as a function of reporter detection rate and reporter cells loaded per pen. The original heatmap was shown in color and the black and white version is shown in FIG. 48.

FIGS. 49A-49B show the distribution of background fluorescence per pen (MeanBackgroundBrightness), brightest reporter cell fluorescence per pen from IgG-secreting OKT3-loaded pens (OKT3 MaxBrightness), and brightest reporter cell fluorescence per pen from IgG-secreting OKT8-loaded pens (OKT8 MaxBrightness). FIG. 49B is a zoomed in view of the fluorescence distributions.

FIGS. 50A-50C show that OKT3 hits, OKT8 hits, and false positive hit rate as a function of signal threshold for pens with >=1 Jurkat reporter cells (FIG. 50A), >=3 Jurkat reporter cells (FIG. 50B), and >=5 Jurkat reporter cells (FIG. 50C) per pen.

### DETAILED DESCRIPTION OF CERTAIN EMBODIMENTS

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

### I. Definitions

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/-5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: µm means micrometer, µm³ means cubic micrometer, pL means picoliter, nL means nanoliter, and µL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25%, or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 µL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 µL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 µL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. The length of the channel is generally defined by the flow path of the channel. In the case of a straight channel, the length would be the “longitudinal axis” of the channel. The “horizontal dimension” or “width” of the channel is the horizontal dimension as observed in a transverse section oriented perpendicular to the longitudinal axis of the channel (or, if the channel is curved, perpendicular to an axis tangential to the flow path of the channel at the plane of the transverse section). The “vertical dimension” or “height” of the channel is the vertical dimension as observed in a transverse section oriented perpendicular to the longitudinal axis of the channel (or, if the channel is curved, perpendicular to an axis tangential to the flow path of the channel at the plane of the transverse section).

For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pats. 6,408,878 and 9,227,200, each of which is herein incorporated by reference in its entirety.

For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pats. 6,408,878 and 9,227,200, each of which is herein incorporated by reference in its entirety.

The direction of fluid flow through the flow region (e.g., channel), or other circuit element (e.g., a chamber), dictates an “upstream” and a “downstream” orientation of the flow region or circuit element. Accordingly, an inlet will be located at an upstream position, and an outlet will be generally located at a downstream position. It will be appreciated by a person of skill in the art, that the designation of an “inlet” or an “outlet” may be changed by reversing the flow within the device or by opening one or more alternative aperture(s).

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated microbeads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or non-selectively. In one non-limiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind non-selectively when binding of structurally different but physicochemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g. perfusing).

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

The defined area can be, for example, a chamber. As used herein, a “chamber” is a region within a microfluidic device (e.g., a circuit element) that allows one or more micro-object(s) to be isolated from other micro-objects located within the microfluidic device. Examples of chambers include microwells, which may be regions etched out of a substrate (e.g., a planar substrate), as described in U.S. Pat. Application Publication Nos. 2013/0130232 (Weibel et al.) and 2013/0204076 (Han et al.), or a region formed in a multi-layer device, such as the microfluidic devices described in WO 2010/040851 (Dimov et al.) or U.S. Pat. Application No. 2012/0009671 (Hansen et al.). Other examples of chambers include valved chambers, such as described in WO 2004/089810 (McBride et al.) and U.S. Pat. Application Publication No. 2012/0015347 (Singhal et al.). Other examples of chambers include the chambers described in: Somaweera et al. (2013), “Generation of a Chemical Gradient Across an Array of 256 Cell Cultures in a Single Chip”, Analyst., Vol. 138(19), pp 5566-5571; U.S. Pat. Application Publication No. 2011/0053151 (Hansen et al.); and U.S. Pat. Application Publication No. 2006/0154361 (Wikswo et al.). Still other examples of chambers include the sequestration pens described in detail herein. In certain embodiments, the chamber can be configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, the chamber can be configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

As used herein, “pen” or “penning” specifically refers to disposing micro-obj ects within a sequestration pen within the microfluidic device. Forces used to pen a micro-object may be any suitable force as described herein such as dielectrophoresis (DEP), e.g., an optically actuated dielectrophoretic force (OEP); gravity; magnetic forces; locally actuated fluid flow; or tilting. In some embodiments, penning a plurality of micro-objects may reposition substantially all the micro-objects. In some other embodiments, a selected number of the plurality of micro-obj ects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and thereafter introduced into a chamber by penning.

As used herein, “unpen” or “unpenning” refers to repositioning micro-objects from within a sequestration pen to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; locally actuated fluid flow; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” can include, consist of, or consist essentially of repositioning micro-objects from a location within a microfluidic device, e.g., a flow region, a microfluidic channel, a chamber, etc., to a location outside of the microfluidic device, such as a well plate, a tube, or other receiving vessel. In some embodiments, exporting a micro-object comprises withdrawing (e.g., micro-pipetting) a volume of medium containing the micro-object from within the microfluidic device and depositing the volume of medium in or upon the location outside of the microfluidic device. In some related embodiments, withdrawing the volume of medium is preceded by disassembling the microfluidic device (e.g., removing an upper layer, such as a cover or lid, of the microfluidic device from a lower layer, such as a base or substrate, of the microfluidic device) to facilitate access (e.g., of a micro-pipetted) to the internal regions of the microfluidic device. In other embodiments, exporting a micro-object comprises flowing a volume of fluid containing the micro-object through the flow region (including, e.g., a microfluidic channel) of the microfluidic device, out through an outlet of the microfluidic device, and depositing the volume of medium in or upon the location outside of the microfluidic device. In such embodiments, micro-object(s) within the microfluidic channel may be exported without requiring disassembly (e.g., removal of the cover of the device) or insertion of a tool into an interior region of the microfluidic device to remove micro-objects for further processing. “Export” or “exporting” may further comprise repositioning micro-objects from within a chamber, which may include a sequestration pen, to a new location within a flow region, such as a microfluidic channel, as described above with regard to “unpenning”. A planar orientation of the chamber(s) with respect to the microfluidic channel, such that the chamber(s) opens laterally from the microfluidic channel, as described herein with regard to sequestration pens, permits easy export of micro-objects that have been positioned or repositioned (e.g., unpenned from a chamber) to be disposed within the microfluidic channel.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

As used herein, an “isolation region” refers to a region within a microfluidic device that is configured to hold a micro-object such that the micro-object is not drawn away from the region as a result of fluid flowing through the microfluidic device. Depending upon context, the term “isolation region” can further refer to the structures that define the region, which can include a base/substrate, walls (e.g., made from microfluidic circuit material), and a cover.

As used herein, “antibody” refers to an immunoglobulin (Ig) and includes both polyclonal and monoclonal antibodies; multichain antibodies, such as IgG, IgM, IgA, IgE, and IgD antibodies; single chain antibodies, such as camelid antibodies; mammalian antibodies, including primate antibodies (e.g., human), rodent antibodies (e.g., mouse, rat, guinea pig, hamster, and the like), lagomorph antibodies (e.g., rabbit), ungulate antibodies (e.g., cow, pig, horse, donkey, camel, and the like), and canidae antibodies (e.g., dog); primatized (e.g., humanized) antibodies; chimeric antibodies, such as mouse-human, mouse-primate antibodies, or the like; and may be an intact molecule or a fragment thereof (such as a light chain variable region (VL), heavy chain variable region (VH), scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen. In some embodiments, antibody fragments may be derivatized to exhibit structural features that facilitate clearance and uptake, e.g., by the incorporation of galactose residues. The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

An antigen, as referred to herein, is a molecule or portion thereof that can bind with specificity to another molecule, such as an Ag-specific receptor. An antigen may be any portion of a molecule, such as a conformational epitope or a linear molecular fragment, and often can be recognized by highly variable antigen receptors (B-cell receptor or T-cell receptor) of the adaptive immune system. An antigen may include a peptide, polysaccharide, or lipid. An antigen may be characterized by its ability to bind to an antibody’s variable Fab region. Different antibodies have the potential to discriminate among different epitopes present on the antigen surface, the structure of which may be modulated by the presence of a hapten, which may be a small molecule.

In some embodiments, an antigen is a cancer cell- associated antigen. The cancer cell-associated antigen can be simple or complex; the antigen can be an epitope on a protein, a carbohydrate group or chain, a biological or chemical agent other than a protein or carbohydrate, or any combination thereof; the epitope may be linear or conformational.

The cancer cell-associated antigen can be an antigen that uniquely identifies cancer cells (e.g., one or more particular types of cancer cells) or is upregulated on cancer cells as compared to its expression on normal cells. Typically, the cancer cell-associated antigen is present on the surface of the cancer cell, thus ensuring that it can be recognized by an antibody. The antigen can be associated with any type of cancer cell, including any type of cancer cell that can be found in a tumor known in the art or described herein. In particular, the antigen can be associated with lung cancer, breast cancer, melanoma, and the like. As used herein, the term “associated with a cancer cells,” when used in reference to an antigen, means that the antigen is produced directly by the cancer cell or results from an interaction between the cancer cell and normal cells.

The terms “nucleic acid molecule”, “nucleic acid” and “polynucleotide” may be used interchangeably and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

As used herein, “B” used to denote a single nucleotide, is a nucleotide selected from G (guanosine), C (cytidine) and T (thymidine) nucleotides but does not include A (adenine).

As used herein, “H” used to denote a single nucleotide, is a nucleotide selected from A, C and T, but does not include G.

As used herein, “D” used to denote a single nucleotide, is a nucleotide selected from A, G, and T, but does not include C.

As used herein, “K” used to denote a single nucleotide, is a nucleotide selected from G and T.

As used herein, “M” used to denote a single nucleotide, is a nucleotide selected from A or C.

As used herein, “N” used to denote a single nucleotide, is a nucleotide selected from A, C, G, and T.

As used herein, “R” used to denote a single nucleotide, is a nucleotide selected from A and G.

As used herein, “S” used to denote a single nucleotide, is a nucleotide selected from G and C.

As used herein, “V” used to denote a single nucleotide, is a nucleotide selected from A, G, and C, and does not include T.

As used herein, “Y” used to denote a single nucleotide, is a nucleotide selected from C and T.

As used herein, “I” used to denote a single nucleotide is inosine.

As used herein, A, C, T, G followed by “*” indicates phosophorothioate substitution in the phosphate linkage of that nucleotide.

As used herein, IsoG is isoguanosine; IsoC is isocytidine; IsodG is an isoguanosine deoxyribonucleotide and IsodC is a isocytidine deoxyribonucleotide. Each of the isoguanosine and isocytidine ribo- or deoxyribo- nucleotides contain a nucleobase that is isomeric to guanine nucleobase or cytosine nucleobase, respectively, usually incorporated within RNA or DNA.

As used herein, rG denotes a ribonucleotide included within a nucleic acid otherwise containing deoxyribonucleotides. A nucleic acid containing all ribonucleotides may not include labeling to indicate that each nucleotide is a ribonucleotide, but is made clear by context.

As used herein, a “priming sequence” is an oligonucleotide sequence which can be part of a larger oligonucleotide but, when separated from the larger oligonucleotide such that the priming sequence includes a free 3′ end, can function as a primer in a DNA (or RNA) polymerization reaction.

### II. Methods for Antibody Discovery

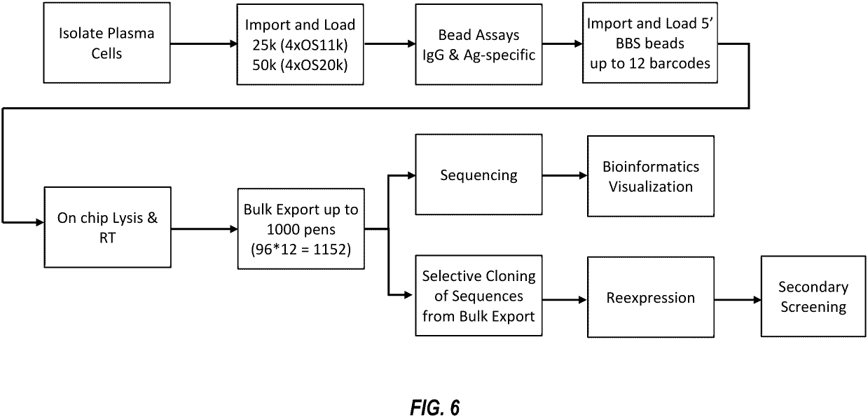

As mentioned above, the time needed in screening cells for lead candidates using macroscale workflows that are typically currently used, significantly adds to the drug development timeline. Thus, it is urgently needed to reduce the time needed for screening cells capable of secreting a desired antibody, to thereby accelerate antibody discovery. FIG. 6 shows a general workflow which is directed to providing acceleration of antibody discovery campaigns. The method includes isolating plasma B cells and importing the cells in a microfluidic device, preferably the microfluidic device as disclosed in the following sections. The cells can be loaded into the channel or chamber of the microfluidic device and cultured individually. In some embodiments, up to 50k single plasma B cells may be loaded. In some embodiments, cells that are determined to be healthy (e.g., viable), substantially healthy, or enriched in a proportion of cells that are healthy, may be introduced preferentially to the chamber(s) of the microfluidic device.

The method may also include conducting binding or functional assays, which can be, but is not limited to bead-based analyses for testing the IgG-antigen specificity of the antibodies secreted in each pen. The method may further include loading nucleic acid capture objects, which may be any nucleic acid capture object at described herein, and performing on-chip lysis, nucleic acid capture and reverse transcription. As explained in more detail in the following sections, barcoded cDNA sequences are generated through these steps by using the capture objects of the present disclosure. The nucleic acid capture objects additionally are labelled, to permit correlation of the binding/functional assay results with the specific nucleic acid isolated from the cell(s) responsible for the assay results. Detection of the labels may be performed at any point during the workflow to identify the label for each capture object in each chamber.

Subsequently, the barcoded cDNA sequences, which are captured on the capture objects and comprise the BCR sequence (i.e., barcoded BCR beads), may be exported to an off-chip culture plate. In some embodiments, barcoded BCR beads from over 1000 pens can be unloaded to a single 96-well plate and permit multiplexing of subsequent processes.

As explained in more detail in the following sections, the capture objects of the present disclosure enable the identification of the origin of the barcoded BCR beads on the 96-well plate. Last, subsequent analyses including sequencing and/or selective cloning of BCR sequences, performing bioinformatics visualization or re-expression of BCR sequences may be performed. Further, in some embodiments, secondary screenings can be conducted. In some embodiments, the method of the present disclosure aims at increasing screening throughput to up to 50k single plasma B cells and over 1000 exports of target B cell receptor (BCR) sequences. Overall, this workflow provides for high throughput antibody discovery methods.

### III. Methods for Identification of Healthy Cells Prior to Importation Into a Chamber.

Identifying healthy cells before importing the cells into a chamber can offer benefits in the methods of the present disclosure. As referred to herein, a healthy cell is a cell demonstrating characteristics of viability, e.g., is a viable cell and has the ability to continue to grow and optionally, produce either biomolecules of interest and/or produce daughter cells having the same capabilities. Disposing into the chambers, e.g., sequestration pens, of the microfluidic device only, substantially only or an increased proportion of healthy cells out of an imported population can increase the likelihood of identifying useful cells/clonal populations thereof. Further, resources used during a biomolecule production development/identification campaign are not expended upon non-viable cells, reducing waste and preserving the use of the pre-defined number of chambers for cells that have some possibility of expressing the biomolecule of interest.

Thus, another aspect of the present disclosure is to identify healthy cells before importing them into the chamber of the microfluidic device. However, identifying healthy cells within a microfluidic device can be difficult, due to the very nature of the small scale of the microfluidic device. Furthermore, for a single cell culture scheme, only a relatively small number of cells may be imported into the device, and staining of such small number of cells may not be able to generate a fluorescent intensity sufficient for meaningful detection. Additionally, for some biomolecule production methods, it may be desirable to not include any sort of dyes or stains to the cells themselves, depending on the downstream uses of the cells. Therefore, it is useful to develop a method of identifying and importing healthy cells which does not rely upon staining every batch of cells to be imported into sequestration pens.

In some embodiments, a staining method can be combined with a brightfield image observation for the purposes of identifying healthy cells.

In some embodiments, identifying a healthy cell can involve the use of a machine learning algorithm to process image data. In some embodiment, the machine learning algorithm is capable of identifying healthy cells without staining. The machine learning algorithm can include a neural network, such as a convolutional neural network. A convolutional neural network (CNN) generally accomplishes an advanced form of image processing and classification/detection by first looking for low level features such as, for example, edges and curves, and then advancing to more abstract (e.g., unique to the type of images being classified) concepts through a series of convolutional layers. A CNN can do this by passing an image through a series of convolutional, nonlinear, pooling (or downsampling, as will be discussed in more detail below), and fully connected layers, and get an output. The output can be a single class or a probability of classes that best describes the image or detects objects on the image. Some examples of CNNs useful in these methods include have been described, for example, International Application Publication No. WO 2019/232473, entitled “Automated Detection and Characterization of Micro-Object in Microfluidic Devices”, filed on May 31, 2019; and in International Application Publication No. WO2018102748, entitled “Automated Detection and Characterization of Micro-Obj ect in Microfluidic Devices”, filed on Dec. 1, 2017, each of which disclosures are incorporated herein by reference.

In some embodiments, a training data used in establishing the CNN model of the present disclosure may include a fluorescent image having the cells of interest stained, a brightfield image having the cells of interest annotated, or combinations thereof. The dyes suitable in the present disclosure may include but are not limited to calcein, zombie violet stain, annexin, acridine orange, propidium iodide, or combinations thereof. Any suitable stain that discriminates between a healthy cell and a dead/dying and/or non-viable cell, as is known to one of skill in the art may be used. In some embodiments, other dyes that are specific to a marker of interest can also be used, for instance, Alexa Fluor® 647 anti-mouse CD138 (Syndecan-1) Antibody (BioLegend), which is highly specific for terminally differentiated live plasma cells and stains CD138 presented on the surface. In some embodiments, two or more dyes can be used in staining a sample to provide cross-reference or verification.

In a particular embodiment, a training data includes images of cells stained with a fluorescent dye in combination with images of the cells under brightfield. A healthy cell can be identified, for example, by observing the morphologies of the cells under brightfield. In some embodiments, a healthy cell, e.g., a viable cell, can be characterized as having a clear cell boundary, good contrast, round shape, or combinations thereof. In some embodiment, a healthy cell can be determined by identifying the unhealthy ones. For instance, an unhealthy cell can be characterized as having debris-like appearance, unclear or different contrast, or combinations thereof. In many embodiments, assessment of viability can be made in a relative manner by comparing cells in a sample. For instance, a healthy cell can have a larger diameter while other cells having a smaller diameter are more likely to be unhealthy/dead or merely cell debris.

In the training regime, cells may be first detected under brightfield and then labeled as live/dead based on the fluorescent intensity. In some embodiment, labeling of live/dead cells is based on a cutoff value of the fluorescent intensity, which can be selected in accordance with the user’s likings or needs.

After training is accomplished with the type of cell under investigation, the method of penning healthy cells using the trained machine learning algorithm may be employed to increase the penning efficiency of healthy cells, and decrease the numbers of non-viable cells penned. In some embodiments, the percentage of healthy cells relative to non-viable cells imported into the chambers, e.g., sequestration pens, after identification by the algorithm, may be improved by about 10%, 15%, 20%, 25%, 30%, 35%, 40%, 50%, 60%, 70%, 80%, 90%, 100%, or more.

### IV. A Method of Assaying For a Specific Binding Interaction Between a First Molecule and a Second Molecule

Binding interactions between a first molecule and a second molecule can be measured in a chamber of a microfluidic chip. The chamber can be any of the chambers described or referenced herein, including a microwell or a sequestration pen, and the assay formats can vary widely. For example, the assay can be a “sandwich” assay in which a surface, such as a bead or an internal surface of a wall of the microfluidic device, is configured to capture and/or present the first molecule; binding of the second molecule is detected via a third molecule that is labeled and capable of binding to the complex formed by the second molecule binding to the first molecule, thereby associating the label of the third molecule with the surface in a detectable manner. In such assays, the second molecule can be produced by a biological cell. The assay surface can be in the chamber (e.g., as described in U.S. Pat. Application Publication No. 2015/0165436 and PCT International Publication No. WO 2010/040851) or proximal to the chamber, such as in a channel that the chamber connects with (e.g., as described in U.S. Pat. Application Publication No. 2015/0151298). Alternatively, the assay can be a diffusion gradient assay in which the second molecule has a label (which can be linked to the second molecule, or can be an intrinsic property of the second molecule, such as auto-fluorescence) and the diffusive properties of the labeled second molecule in the presence of the first molecule can be monitored, e.g., as described in PCT International Publication No. WO 2017/181135. In such assays, the first molecule can be produced by a biological cell. Still other assays can feature a blocking interaction in which a molecule of interest binds to the first molecule and thereby blocks an interaction of the first molecule with the second molecule. In such assays, the molecule of interest can be produced by a biological cell, and the second molecule can comprise a label. As with the sandwich assays, the blocking assays can feature the first molecule bound to a surface. The surface can be located, for example, in the chamber or a region proximal to the chamber, such as a channel. Examples of blocking assays are described below and elsewhere herein, including in the examples and in the claims.

In-channel Binding Assay. In some embodiments, a method of assay for a specific binding interaction between a first molecule and a second molecule is provided. The method can be performed within a microfluidic device having a channel and a chamber, such as a microwell or a sequestration pen, fluidically connecting to the channel. The method can include: introducing each of a plurality of biological cells into a respective one of a plurality of chambers; incubating the biological cells and allowing the biological cell to produce and/or secrete a molecule of interest; introducing a micro-object including a plurality of first molecules into the channel; and monitoring an accumulation of the molecule of interest on the micro-object.

In some embodiments, monitoring an accumulating of the molecule of interest on the micro-object including introducing a third molecule that is labeled and capable of binding to the complex formed by the molecule of interest binding to the first molecule, thereby associating the label of the third molecule with the accumulation of the molecule of interest on the micro-object. Some aspects of an in-channel assay using micro-objects comprising beads having a plurality of first molecules are further described in an International Application filed on Oct. 22, 2014, and published as International Publication WO2015/061497.

In some embodiments, introducing a micro-object including a plurality of first molecules, e.g., a reporter cell, into the channel including introducing a plurality of the micro-objects and allowing the plurality of the micro-objects fill the channel at a density. In some embodiments, an optimal density is such that nearly the entire channel is filled with the micro-objects. A density that is below optimal might result in a sparse number of micro-objects in the channel and an undersampling of the secreted molecule of interest, making unambiguous identification of the secreting chamber difficult. On the other hand, an overly concentrated density might lead to higher risk of channel blockages, poor uniformity across the chip, and might lead to the micro-objects getting pushed into the chambers. In some embodiments, the optimal density can vary depending on the size of the micro-objects introduced. In certain embodiments, the micro-objects are biological cells, and the density can be from about 10⁷ to 10⁹, or about 10⁸ to 2×10⁸ cells/mL. In some embodiments, the micro-object including the plurality of first molecules, e.g., reporter cells, may be cells that may be cells that culture in suspension. In other embodiments, adherent cell types may be used as reporter cells when detachment protocols are used. For example, adherent CHO cells may be successfully used when a detachment protocol may include: culturing to confluence prior to importation, e.g. not exceeding confluence; and treating with a detachment reagent such as Accutase (ThermoFisher Scientific, A1110501), TrypLE or the like, e.g. treatment at about 22° C. for 10 min with no agitation. The adherent CHO cells were then successfully importable as monodisperse cells and the target cell densities were achieved. Specific detachment protocols may be determined as needed for other cell types. The preparatory culture density may be varied, e.g., less than about 100% confluent, less than about 90% confluent, less than about 80% confluent, less than about 60% confluent, or less than about 50% confluent. The detachment reagent may be varied. The duration of the detachment treatment may be varied, e.g., from about 5 min to about 1h, about 10 min, about 15 min, about 20 min, about 30 min, about 45 min, about 60 min or any value therebetween. In some embodiments, agitation is not employed. In yet other embodiments, the cells may be agitated during the detachment treatment. Temperature may be varied in order to successfully detach the cells, and may be varied from about 15° C. to about 36° C., about 10° C. to about 40° C., or any temperature therebetween. Filtration through a cell strainer may be useful to remove cell clumps or other large debris, and may be performed before concentrating the cells to target import concentration. The cells may be concentrated by centrifuging at 400 x g for 5 min, and resuspended to the desired concentration. The third molecule, which is labeled and capable of binding to the complex formed by the molecule of interest binding to the first molecule, e.g., a labelled antibody, may be added to the media when resuspending the cells.

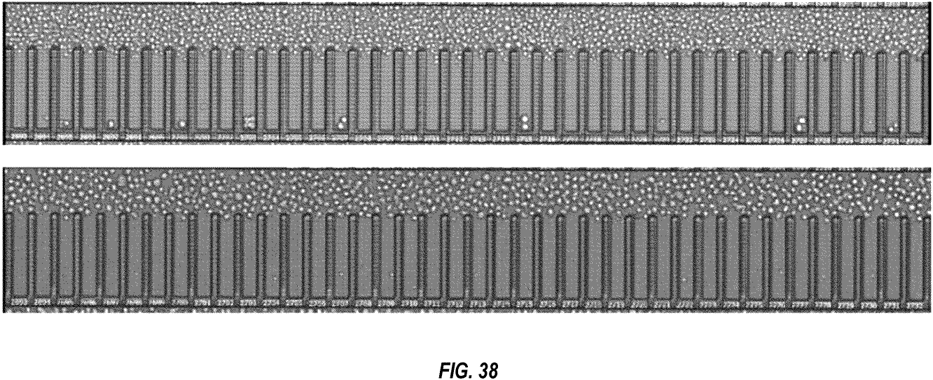

In a specific embodiment, as shown in FIG. 38, Jurkat cells (upper) and K562 cells (lower) were used as the micro-objects at a density of 1.7×10^8 cells/mL and 1×10^8 cells/mL respectively. The figures show that the channels were nearly filled by the cells at an acceptable extent for the in-channel binding assays.

In some embodiments, the first molecule and/or the molecule of interest can be a protein. The protein can be, for example, a cell surface protein or an extracellular protein. The protein can be a modified protein, such as a glycosylated protein, a lipid-anchored protein, or the like. In some embodiments, the molecule of interest can specifically bind to the first molecule. In certain embodiments, the first molecule and molecule of interest can be an antigen-antibody pair. For example, the biological cell can be a B cell producing an antibody of interest (i.e., molecule of interest) and the first molecule presented on the surface of the micro-objects can be an antigen or epitope of the produced antibody. In some embodiments, the third molecule can be a secondary antibody binding to the produced antibodies (i.e., the secreted second molecule), and the detection thereof is associated with the binding of the first molecule and the molecule of interest.

In certain embodiments, the micro-object can be one or more beads or cells that express the first molecule. If a cell, the cell can express the first molecule naturally or can be genetically modified (e.g., stably or transiently transfected) to express the first molecule. If a bead, the bead can be created by conjugating the first molecule on its surface.

Blocking Assay. In some embodiments, a method of assaying for inhibition of a specific binding interaction between a first molecule and a second molecule is provided. The method can be performed within a microfluidic device having a chamber, such as a microwell or a sequestration pen, and can include: introducing each of a biological cell and a micro-object that comprises a plurality of first molecules into the chamber of the microfluidic device; incubating the biological cell in the presence of the micro-object and allowing the biological cell to produce and/or secrete a molecule of interest; introducing the second molecule into the chamber, wherein the second molecule is bound to a detectable label (or intrinsically produces a signal, such as auto-fluorescence); and monitoring an accumulation of the second molecule on the micro-object. One or more micro-objects can be loaded with the cell into the chamber. An absence or diminishment of accumulation of the second molecule on the one or more micro-objects indicates that the molecule of interest produced by the cell inhibits binding of the first molecule to the second molecule.

In some embodiments, monitoring an accumulation of the second molecule on the micro-object comprises comparing the accumulation to that observed on a control micro-object in the presence of a positive control molecule of interest and/or a negative control molecule of interest. In other embodiments, monitoring an accumulation of the second molecule on the micro-object comprises comparing the accumulation to that observed on a control micro-object in the absence of a control molecule of interest.

As used herein, the term “diminishment” indicates lower accumulation compared to that observed in one or more control chambers. In some embodiments, the control chamber can be a negative control chamber. Examples of negative control chambers may include, but are not limited to, chambers containing a control micro-object and a negative control cell. The negative control cell can be a cell producing a molecule that is known not to bind the first molecule or the second molecule, or a cell known not to produce a molecule of interest. Other examples of negative control chambers include chambers containing a control micro-object by itself (i.e., with no control cell present). Accordingly, in some embodiments, monitoring an accumulation of the second molecule on the micro-object comprises comparing the accumulation to that observed on a control micro-object incubated in the presence of one or more or no negative control cells.

In some embodiments, the control chamber can be a positive control chamber. Examples of positive control chambers may include, but are not limited to, chambers containing a control micro-object and a positive control cell. The positive control cell can be a cell producing a molecule that is known to bind the first molecule or the second molecule and thereby inhibit binding of the first molecule to the second molecule.

Control cells (e.g., positive or negative control cells) may be introduced into the same microfluidic device as the biological cell that is capable of producing a protein of interest, or into a different microfluidic device. Control cells may be introduced into the microfluidic device at a same period or a different time period.

In some embodiments, the method is performed within a microfluidic device having a plurality of chambers, and monitoring an accumulation of the second molecule on the micro-object comprises comparing the accumulation to that observed in one or more other chambers of the plurality where one or more control cells are introduced. A control cell in another chamber may be introduced intentionally (i.e., when the control cell is known to be a positive or negative control cell) or control cells may be identified from the pool of introduced cells based on the fact that a user will expect that not all of the introduced cells will produce a molecule of interest capable of impacting the accumulation of the second molecule on the micro-object. In some embodiments, monitoring an accumulation of the second molecule on the micro-object comprises comparing the accumulation to that observed in one or more other chambers of the plurality where no cell is introduced.

In some embodiments, comparison to a single control chamber is sufficient (e.g., a control chamber having a single well-characterized control cell or no control cell at all). In other embodiments, the method includes comparison to a plurality of control chambers. For example, the comparison can comprise comparing accumulation of the second molecule on the micro-object with a statistical measure of the accumulation of the second molecule on the control micro-objects in the plurality of control chambers (e.g., an average accumulation or a level of accumulation that is one, two, or three standard deviations below the average accumulation on the control micro-objects in the plurality of control chambers). Alternatively, the comparison can comprise comparing accumulation of the second molecule on the micro-object with the minimum accumulation (or maximum accumulation) of the second molecule on the control micro-objects in the plurality of control chambers.

In some embodiments, the method is performed within a microfluidic device having a microfluidic channel and a plurality of chambers, and monitoring an accumulation of the second molecule on the micro-object comprises comparing the accumulation to that observed in an area outside the chamber where the cell is introduced (e.g., in the microfluidic channel), in one or more other chambers of the plurality where one or more control cells are introduced, or in one or more chambers where no cell is introduced.

In certain embodiments, the first molecule and/or the second molecule can be a protein. The protein can be, for example, a cell surface protein or an extracellular protein. The protein can be a modified protein, such as a glycosylated protein, a lipid-anchored protein, or the like. In certain embodiments, the first molecule and second molecules can be a receptor-ligand pair. The “ligand” used herein refers to a molecule that has a region, structure, or motif that can be recognized and bound specifically by a receptor at a certain level of affinity. In some embodiments, the level of affinity is high enough to form and maintain a receptor-ligand complex during the operation of the blocking assay of the present disclosure but is lower than the level of affinity of the molecule of interest to the ligand or the receptor. In some embodiments, the first molecule is a receptor molecule, and wherein the second molecule is a ligand that specifically binds to the receptor molecule. For example, the first molecule can be a growth factor receptor, a cytokine receptor, a chemokine receptor, an adhesion receptor (e.g., an integrin or a cell adhesion molecule (CAM)), an ion channel, a G protein-coupled receptor (GPCR), or a fragment retaining activity of its respective full length biomolecule of any of the foregoing; and the ligand can be a growth factor, a cytokine, a chemokine, an adhesive ligand, an ion channel ligand, a GPCR ligand, a viral protein (e.g., a viral coat or capsid protein, such as a fusion protein), or a fragment retaining activity of its respective full length biomolecule of any of the foregoing. In some embodiments, the first molecule is a ligand, and the second molecule is a receptor that is specifically bound by the ligand as exemplified above. In certain embodiments that the second molecule is a receptor, the receptor can be a receptor molecule anchored on an object such as a cell, a bead, a lipid particle. Alternatively, when the second molecule is a receptor, the receptor molecule may be a soluble receptor molecule. The receptor molecule can be manufactured by chemical synthesis or a semisynthetic process.

In certain embodiments, the one or more micro-objects can be one or more beads or cells that expresses the first molecule. If a cell, the cell can express the first molecule naturally or can be genetically modified (e.g., stably or transiently transfected) to express the first molecule.

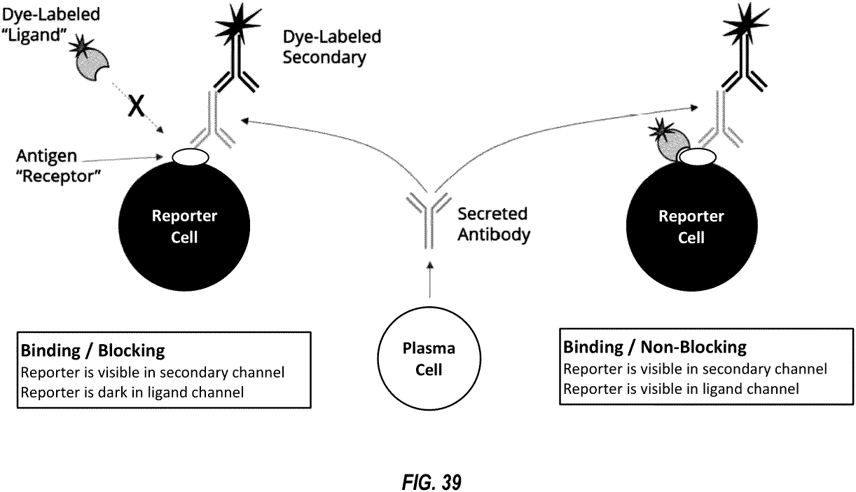

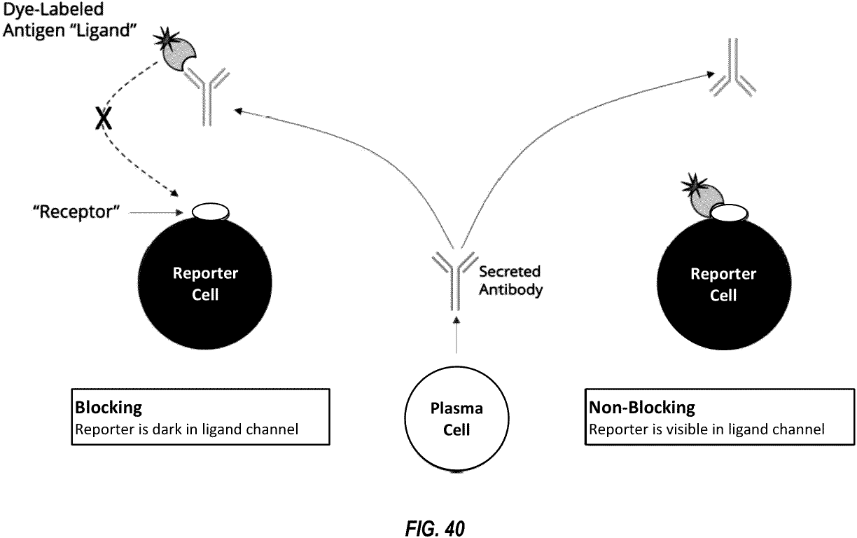

The blocking assays described herein can be a receptor blocking assay or a ligand blocking assay. In an exemplary receptor blocking assay (FIG. 39), the targeted antigen (i.e., the first molecule) may be located on the surface of the reporter cell (i.e., the micro-object), and the secreted antibody binds to this surface-bound antigen “receptor”, potentially blocking the binding of the dye-labeled, soluble “ligand” (i.e., the second molecule). Conversely, in a ligand blocking assay (FIG. 40), the targeted antigen (i.e., the second molecule) may be in solution, and the secreted antibody binds to this antigen “ligand”, potentially blocking its binding to the receptor (i.e., the first molecule) of the reporter cell. In either design, if the secreted antibody is an effective blocker, little or no dye-labeled ligand (i.e., second molecule) will bind to the reporter surface, and the reporter cell will be dark in the fluorescence channel associated with the ligand (the “ligand channel”). If the secreted antibody is non-blocking, the dye-labeled ligand will bind to and accumulate on the reporter surface, and the reporter will be visible in the fluorescence channel associated with the ligand.

In some embodiments of receptor blocking assays, an optional secondary antibody (i.e., the third molecule), labeled with a dye different than that of the ligand, may be included to confirm binding of the secreted antibody to the reporter (FIG. 39). In this design, if the secreted antibody both binds to the receptor and blocks ligand binding, the reporter will be visible in the secondary channel and dark in the ligand channel. However, if the secreted antibody binds the receptor but does not block ligand binding, the reporter cell will be visible in both the secondary and ligand channels. In order to determine binding in the ligand blocking assay design, a separate in-channel assay should be performed.

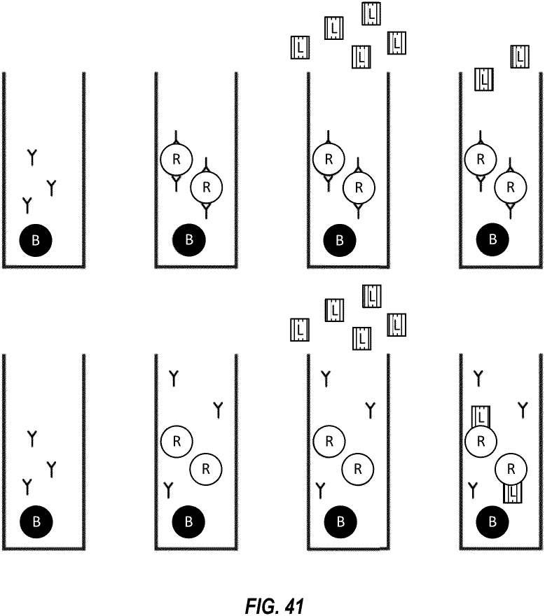

In some embodiments of receptor blocking assays (FIG. 41), the biological cells, producing a molecule of interest, labeled as “B” may be penned first, followed by introduction of micro-objects including first molecules, e.g., reporter micro-objects, which may be beads or cells. The upper and low rows of FIG. 41 represent timepoints, e.g., each chamber, left to right, along each row, for two different types of receptor blocking assays. As shown in the upper row of FIG. 41, an exemplary embodiment is shown where the molecules of interest, e.g., an antibody produced by cell “B”, bind to the reporter micro-objects and block the ligands “L” from binding to the reporter micro-objects “R”. Upon introduction to the chamber, the cells (“B”) producing the molecule of interest, e.g., an antibody in this instance, are shown in the first (left-hand side of the upper row of FIG. 41) exemplary chamber. After introduction of the reporter micro-objects, labelled “R” (second from left, upper row of FIG. 41), the micro-objects may then be incubated with the secreting biological cells, permitting binding of the molecule of interest to the reporter micro-object. In the third chamber of the upper row of FIG. 41 is shown introduction of the dye labeled ligands, labelled “L” (i.e., the second molecule). In this embodiment, the secreted antibody is capable of binding to and saturating the receptor (i.e., the first molecule, which may include an antigen binding site) on the micro-objects, blocking the ligand from binding. The labelled ligand therefore does not label the reporter molecule and accumulation of signal on the reporter micro-object is diminished or eliminated.

In the lower row of FIG. 41, is shown a different embodiment. The secreting biological cell “B” is introduced to the chamber (first chamber, left hand of lower row of FIG. 41) and produces the molecule of interest, e.g., an antibody. In the second chamber of lower row of FIG. 41, the reporter micro-objects “R” including the first molecules are introduced, as before. In the third chamber of the lower row of FIG. 41, the ligand “L” is introduced and is capable of binding to the reporter micro-object and blocking the molecule of interest from binding (or from stably binding) to the first molecules of reporter micro-object “R”, and in the fourth chamber, is shown the time point, where the ligand “L” (e.g., the second molecule) has accumulated on the reporter micro-object “R”, binding to the first molecule associated thereto, and accumulation of signal is observed.

The two cell types may be cultured together using a Pulse Culture operation consisting of alternating intervals of zero-flow incubation and short periods of chip flushing designed to allow binding of secreted antibodies and minimize pen-to-pen diffusion. Flush volume, flush rate, and incubation duration are tunable parameters and can be adjusted based on user selections. After this period of pulsed culture, typically 30 min, a solution containing dye-labeled ligand is imported and allowed to diffuse into the pens, where it can bind to unblocked reporter cells. Finally, flushes are performed to wash out unbound ligands and images are acquired to assess blocking.

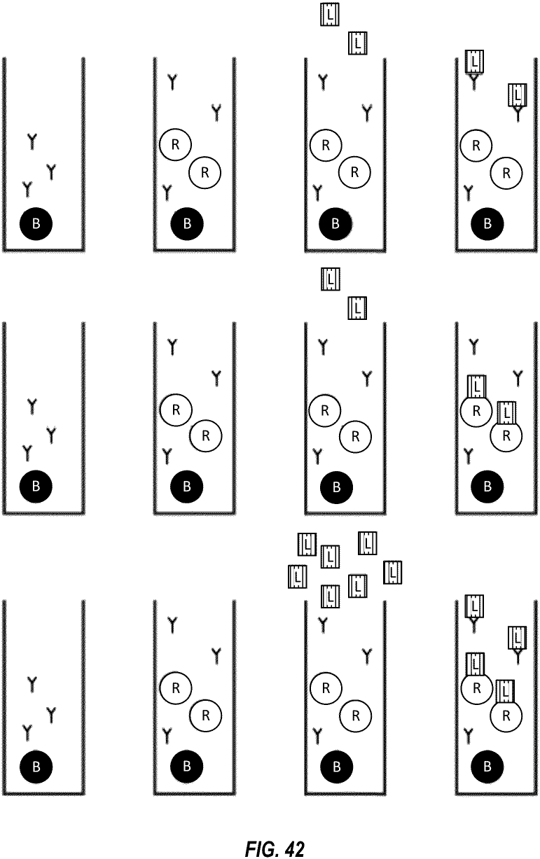

In some embodiments of ligand blocking assays (FIG. 42), the biological cells and the micro-objects may be penned sequentially. In this type of assay, rather than ensuring that reporter cell receptors, e.g., first molecules, are saturated, since the ligand is, for instance an antigen, a Pulse Culture incubation period can be performed to let the micro-objects producing a molecule of interest “B”, e.g., plasma cells, recover and resume secreting prior to importing the antigen “ligand”. Once the B cells recover and resume secreting antibodies of interest, dye-labeled ligand (i.e., the second molecule, which is an antigen in this embodiment) is introduced and allowed to diffuse into the pens. If not blocked by the secreted antibody, the ligand can bind to the reporter cells “B”. Blocking of the ligand by the secreted antibody (molecule of interest) prevents ligand binding to the reporters (e.g., the micro-objects including the first molecule). In FIG. 42, the chambers, left to right of each row, demonstrate successive timepoints during a particular version of a ligand blocking assay. Labels are as for FIG. 41. In the top row, a ligand blocking assay is shown where the secreted molecules of interest, e.g., an antibody as shown here, bind to the introduced ligand, e.g., second molecule. Thus, accumulation of signal on the first molecules, e.g., a receptor molecule, of reporter micro-object “R” is diminished or inhibited. In the second embodiment, shown in the second row of FIG. 42, the secreted molecules of interest, e.g., antibody, are non-blocking and do not prevent accumulation of signal on the first molecules of the reporter micro-object “R”. Signal is therefore observed to accumulate on the reporter micro-objects including the first molecules. Thus, the secreted molecules of interest, e.g., an antibody, does not bind to the first molecules, e.g., a receptor of the reporter micro-object, stably or with sufficient affinity to prevent displacement by the ligand, e.g., second molecule. The bottom row of FIG. 42 represents an embodiment, where, while the secreted molecule of interest, e.g., an antibody, may be capable of binding to the first molecule of the reporter micro-objects “R”, ligand “L” is introduced in a high concentration, sufficiently high that its concentration exceeds that of the secreted molecule of interest, and thus can both bind to the secreted molecule of interest and well as first molecules of the reporter micro-objects. Thus, signal may accumulate on the reporter micro-objects, and may lead to a false negative result.

Ligand titration and incubation timing. In some embodiments, ligand concentration (i.e., the concentration of the second molecule) can be optimized prior to running a blocking assay of the present disclosure. Too little ligand can result in low signal accumulation on the reporter cell, making discrimination between blocking and non-blocking antibodies difficult. Too much ligand can result in a large fraction of unbound ligand, resulting in higher background and potentially lower blocking assay sensitivity. In the case of the receptor blocking assay, excessively high ligand concentration can result in otherwise blocking antibodies being displaced by the concentrated ligand due to competition. In the ligand blocking design, there may not be enough secreted antibody to block all of a high concentration of ligand. In certain embodiments, a concentration of the second molecule is at least 5 nM, from about 5 to about 30 nM, at least 6 nM, or from about 6 nM to about 30 nM.

Ligand Binding Specificity. In some embodiments, confirmation that ligand binding is specific to the surface-expressed receptor (i.e., the first molecule of the micro-object), with minimal non-specific binding to the reporter may be performed. In the case of endogenously expressing reporter cells, a knockout cell line, where receptor expression is eliminated, can serves as a useful negative control to confirm specificity of ligand binding. Similarly, for transfected reporter cell lines, both the parental and transfected cells can be screened to confirm that ligand binding is specific to transfection, with minimal binding to the parental cell line. This measurement of specificity can be performed off-chip using standard flow cytometry methods, or on-chip by importing and penning reporter cells and negative control reporter cells into different regions of the chip, followed by importing and incubating with the dye-labeled ligand. During incubation, the chip may be regularly imaged in the fluorescence channel of the ligand to detect signal accumulation on the reporter cell populations. The intensity difference between the two reporter populations may be clearly discernible, with little or no detectable signal on the negative control reporters.

Reporter heterogeneity. In some embodiments, heterogeneity of the reporter cell (e.g., the micro-object) may be performed for in-pen blocking assays since only a few reporter cells are introduced into any single pen. Consistent with cell-binding assays, an ideal reporter cell population would have a high surface expression of receptor and low receptor expression variation such that each cell has nearly the same level of expression. In such a case, all the reporter cells would bind the same amount of dye-labeled ligand in the absence of non-blocking antibodies, and all cells would be equally bright in the imaging channel of the ligand. In the presence of blocking antibodies, little to no dye-labeled ligand would bind the reporter, and it would appear “dark” in the ligand imaging channel. However, if the reporter population has a large fraction of cells with low receptor surface concentration, this subpopulation may appear “dark” even in the absence of blocking antibodies, leading to an increase in false positive blocking hits, especially with few reporter cells in each pen.

In certain embodiments, the micro-object is a cell, which is from a transfected cell line. In some embodiments, the transfected cell line may be stably or transiently transfected to express the plurality of first molecules. In certain embodiments, at least 60%, at least 65%, at least 70%, at least 75%, at least 80%, at least 85%, at least 90%, at least 95% or more of the cells in the transfected cell line may express the first molecules at detectable levels. In certain embodiments, in order to reduce the false positive rate, each pen is introduced with at least two or at least three micro-objects, where each micro-object includes the first molecule.

The blocking assays described herein can be coupled with any of the other assays described herein, including the sandwich and/or diffusion gradient assays. Cells identified as producing a molecule of interest that blocks the interaction between the first and second molecules can be further analyzed, for example, using the 5′ barcoding methods described herein.

### V. Methods of Capturing the 5′ Ends of RNA and Barcode Identification

Provided herein are methods of capturing the 5′ ends of RNA. Also provided here are methods of providing one or more 5′ barcoded cDNA sequences by reverse transcribing RNA captured from a biological cell.

In some embodiments, the methods comprise providing a biological cell within a chamber. The cell may be provided within a microwell of a microfluidic device. The cell may be provided within a sequestration pen located within an enclosure of a microfluidic device. In some embodiments, the methods comprise disposing the biological cell within a sequestration pen located within an enclosure of a microfluidic device. In some embodiments, a single capture object is provided in the chamber. The biological cell, reagents, time period (and optionally other conditions), sequestration pens, and microfluidic devices may be any of those described herein.

In some embodiments, the methods comprise providing a capture object within the chamber. Further embodiments of disposing one or more biological cells and/or capture objects within the chamber (e.g., microwell or sequestration pens of the microfluidic device) are described in the section entitled “Microfluidic Device and System”.

A capture object described herein comprises a label, a plurality of first oligonucleotides, and a plurality of second oligonucleotides. In some embodiments, each first oligonucleotide of the plurality comprises a barcode sequence and a sequence comprising at least three consecutive guanine nucleotides at a 3′ end. In some embodiments, each first oligonucleotide of the plurality comprises a barcode sequence and a sequence comprising three consecutive guanine nucleotides at a 3′ end. In some embodiments, each first oligonucleotide of the plurality comprises a barcode sequence and a sequence comprising at least three consecutive guanine nucleotides at a 3′ end, and each second oligonucleotide of the plurality comprises a capture sequence. In some embodiments, each first oligonucleotide of the plurality further comprises a priming sequence that corresponds to a first primer sequence. In some embodiments, each second oligonucleotide of the plurality further comprises a priming sequence that corresponds to a second primer sequence. In some embodiments, the first oligonucleotide comprises a first priming sequence that corresponds to a first primer sequence and wherein the second oligonucleotide comprises a second priming sequence that corresponds to a second primer sequence. In some embodiments, the first and second primer sequences are the same. In some embodiments, the first oligonucleotide and the second oligonucleotide are individually linked to the capture object, e.g., the first oligonucleotide is part or all of a first molecule linked to the capture object and the second oligonucleotide is part or all of a second molecule linked to the capture object, where the first and second molecules are different and independently attached to the capture object.

In some embodiments, the methods comprise lysing the biological cell. In some embodiments, the methods comprise allowing RNA molecules released from the lysed biological cell to be captured by the capture sequences of the plurality of second oligonucleotides, e.g., comprised by a capture object. In some embodiments, the methods comprise lysing the biological cell and allowing RNA released from the lysed biological cell to be captured by the capture sequences of the plurality of second oligonucleotides, thereby forming captured RNA. The capture object, capture sequences, priming sequences, and lysis procedures may be any of those described herein.

In some embodiments, lysing the biological cell is performed such that a plasma membrane of the biological cell is degraded, releasing cytoplasmic RNA from the biological cell. In some embodiments, the lysing reagent may include at least one ribonuclease inhibitor. An exemplary lysis reagent is commercially available in the Single Cell Lysis Kit, Ambion Catalog No. 4458235. This reagent can be flowed into the microfluidic channel of a microfluidic device and permitted to diffuse into sequestration pens, followed by a suitable exposure period (e.g., 10 minutes; shorter or longer periods may be appropriate depending on cell type, temperature, etc.). Lysis can be stopped by flowing in an appropriate stop lysis buffer, e.g., from the Single Cell Lysis Kit, Ambion Catalog No. 4458235 and incubating for an appropriate time. Similar results can be obtained using other lysis buffers, including but not limited to Clontech lysis buffer, Cat #635013, which does not require a stop lysis treatment step. Released mRNA can be captured by a capture object present within the same sequestration pen.

In some embodiments, the methods comprise reverse transcribing captured RNA. In some embodiments, one or more barcoded cDNA sequences is produced. In some embodiments, each cDNA sequence comprises an oligonucleotide sequence complementary to a corresponding one captured RNA covalently linked to the reverse complement of the barcode sequence of the first oligonucleotide. In some embodiments, the methods comprise reverse transcribing the captured RNA, thereby producing one or more barcoded cDNA sequences, each comprises an oligonucleotide sequence complementary to a corresponding one captured RNA covalently linked to the reverse complement of the barcode sequence of the first oligonucleotide. Reverse transcribing RNA molecules may be performed according to any appropriate procedure described herein. In some embodiments, the capture sequence binds to, and thereby captures, RNA and primes transcription from the captured RNA. In some embodiments, a reverse transcription (RT) polymerase transcribes the captured RNA.

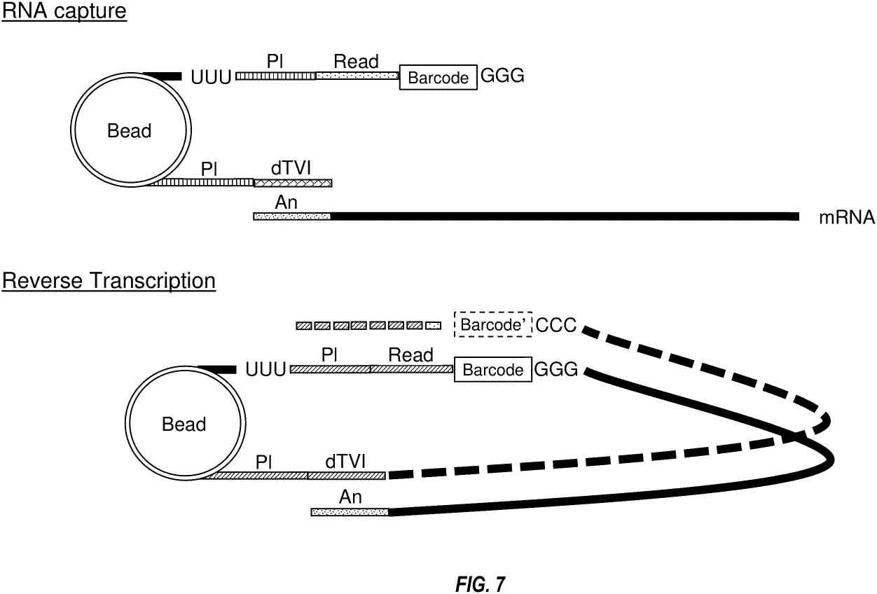

FIG. 7 shows a schematic representation of an exemplary process. A biological cell may be placed within a sequestration pen within a microfluidic device. A capture object, which may be configured as any capture object described herein, may be disposed into the same sequestration pen, which may be performed before or after disposing the cell into the sequestration pen. The cell may be lysed using a lysis reagent which lyses the outer cell membrane of cell but not the nuclear membrane. A lysed cell results from this process and releases RNA. The second oligonucleotide of capture object includes a priming sequence, which has a priming sequence (e.g., corresponding to P1 primer) and a capture sequence, which in this case includes a PolyT sequence which can capture the released nucleic acid having a Poly A sequence at its 3′ end. The capture sequence captures the released nucleic acid. Next, the second oligonucleotide is extended through reverse transcription from the released nucleic acid while in the presence of template switching oligonucleotide. As the captured RNA is transcribed, the transcript is extended to include several C (cytosine) nucleotides, which brings the end of the RNA distal to the PolyA tail into alignment with the rGrGrG terminus of the oligonucleotide bearing the barcode (including a TSO). Identification of the barcode may be performed, using any of the methods described herein either before RNA capture to the barcoded beads; before reverse transcription of the RNA captured to the beads, or after reverse transcription of the RNA on the bead. In some embodiments, identification of the cell specific barcode may be performed after reverse transcription of RNA captured to the bead. After both reverse transcription and identification of the barcode of the capture object has been achieved, the cDNA captured by the capture object is exported out of the chamber into e.g., a common receptacle. A plurality of cDNA capture objects may be exported at the same time and the amplification may be performed, using a common amplification primer (e.g., P1 primer).

In some embodiments, the methods further comprise identifying the barcode sequence of the plurality of first oligonucleotides while the capture object is located within the chamber. Identifying can include detecting the barcode with one or more labeled antisense oligos (e.g., as described in U.S. Pat. Application Publication No. 2019/0345488).

In some embodiments, identifying the barcode comprises detecting fluorescence emitted from the label, which may be an integral part of the capture object or an extrinsic label capable of binding to another molecule (e.g., an oligonucleotide) on the surface of the capture object. In some embodiments, the label comprises one or more fluorophores. In some embodiments, the label comprises a single fluorophore. In some embodiments, the label comprises multiple fluorophores, with each fluorophore present at one or more levels, resulting in unique combinations of fluorophores and fluorophore levels that constitute unique labels. The detectable label can be, for example, a fluorescent label, such as, but not limited to a fluorescein, a cyanine, a rhodamine, a phenyl indole, a coumarin, or an acridine dye. Some non-limiting examples include Alexa Fluor dyes such as Alexa Fluor® 647, Alexa Fluor® 405, Alexa Fluor® 488; Cyanine dyes such as Cy® 5 or Cy® 7, or any suitable fluorescent label as known in the art. Any set of distinguishable fluorophores may be selected to be present on hybridization probes flowed into the microfluidic environment for detection of the barcode, as long as each dye’s fluorescent signal is detectably distinguishable. Alternatively, the detectable label can be a luminescent agent such as a luciferase reporter, a lanthanide tag or an inorganic phosphor, or a Quantum Dot, which may be tunable and may include semiconductor materials. Other types of detectable labels may be incorporated such as FRET labels which can include quencher molecules along with fluorophore molecules. FRET labels can include dark quenchers such as Black Hole Quencher® (Biosearch); Iowa Black™ or dabsyl. The FRET labels may be any of TagMan® probes, hairpin probes, Scorpion® probes, Molecular Beacon probes and the like. In some embodiments, barcodes of capture objects may be identified or deconvolved as follows. Capture objects are initially detected by brightfield imaging. Fluorescence is then measured in a plurality of fluorescence channels (e.g., two, three, or four channels, such as channels corresponding to two, three, or four of FITC, Cy5, DAPI, and Texas red (TRED)) with a plurality of measurements being taken in each channel.

In some embodiments, detecting the label of the capture object may include determining a signal observed for the capture object in more than one fluorescence channel, e.g., each distinct label may be determined by observing/imaging a unique signature of intensities across two, three or four fluorescence channels (such as FITC, Cy5, DAPI and TRED). Detecting each distinct label yields the previously paired identity of the barcode associated with that distinguishable label. In some embodiments, the distinguishable label is integral to the capture object as described above. No matter what type of label of the capture object is, determining the identity of the label permits determining and correlating the origin pen of the cell with the sequencing results obtained after nucleic acid capture and sequencing, which may be performed via any suitable method, including a massively parallel sequencing method.

In some embodiments, the barcode sequence of the first oligonucleotide corresponds to the label of the capture object. For example, there can be a one-to-one relationship between the barcode sequence of the first oligonucleotide and the label of the capture object. In one non-limiting example, the barcode sequence of the first oligonucleotide corresponds to the label of the capture object, which is integral to the capture object, e.g., an integral fluorescent, visible or luminescent color of the capture object. In some embodiments, the barcode sequence of the first oligonucleotide is the label of the capture object.

In some embodiments, one or more fluorophores are directly disposed on the capture object itself. In some embodiments, one or more fluorophores are inked via an oligonucleotide that binds to the barcode sequence or the reverse complement of the barcode sequence.

In some embodiments, the first oligonucleotide comprises one or more uridine nucleotides 5′ to the barcode sequence and, if present, the first priming sequence. In some embodiments, the first oligonucleotide comprises three uridine nucleotides 5′ to the barcode sequence and, if present, the first priming sequence. In further embodiments, the one or more uridine nucleotides are adjacent to or comprise the 5′-most nucleotide(s) of the first oligonucleotide. In some embodiments, reverse transcribing the captured RNA is performed in the presence of an enzyme that cleaves a sequence containing one or more uridine nucleotides (e.g., a USER enzyme).

In some embodiments, each of the one or more barcoded cDNA sequences is associated with the capture object. In some embodiments, the one or more barcoded cDNA sequences are produced in the chamber.

In some embodiments, the methods further comprise exporting the capture object from the chamber. Exporting the plurality of the capture objects may include exporting each of the plurality of the capture objects individually. In some embodiments, the method may further include delivering each capture object of the plurality to a separate destination container outside of the microfluidic device. The destination container may be a common receptacle, a cell culture flask, dish, petri dish, multi-well plate, or the like.

In some embodiments, the methods further comprise storing the one or more barcoded cDNA sequences. In some embodiments, the one or more barcoded cDNA sequences are stored at a temperature at about 4° C.

In some embodiments, the methods further comprise amplifying the one or more barcoded cDNA sequences. In some embodiments, amplifying the one or more barcoded cDNA sequences comprises using a single primer (e.g., a P1 primer). In other embodiments, amplifying the one or more barcoded cDNA sequences comprises using a pair of primers (e.g., P7 and P5 primers).

Where applicable, providing the capture object, providing the biological cell, lysing/transcribing captured RNA, and identifying the barcode sequence, of methods disclosed herein can be performed in the order in which they are written or in other orders, with the limitation that the rearrangement of the order of these activities does not violate logical order (e.g., transcribing before lysing, and so on). As an example, identification of the barcode sequence can be performed after providing the biological cell, after lysing the biological cell, or after transcribing the captured RNA. Likewise, the step of providing the capture object in the chamber can be performed after providing the biological cell in the chamber.

### VI. Methods of Demultiplexing a Pool of Exported cDNA and Preparation of Expression Construct Therefrom

In some embodiments, the methods further comprise performing the method on a plurality of biological cells provided in a corresponding plurality of chambers. In some embodiments, a plurality of capture objects are provided to the plurality of chambers, each capture object of the plurality having (i) a unique label selected from a plurality of unique labels (e.g., at least 12, 14, 16, 18, 20, 25, 30, 40, 50, 60, 70, 80, 90, 100, 150, 200, 250, 500, 1000, or more different labels, or a number of labels falling within a range defined by any two of the foregoing values), and (ii) a plurality of first oligonucleotides having a barcode sequence corresponding to the unique label.

In some embodiments, the methods further comprise exporting the plurality of capture objects into a common receptacle; and amplifying the one or more barcoded cDNA sequences from each capture object of the plurality, thereby producing a plurality of barcoded cDNA sequences, each barcoded cDNA sequence having a barcode sequence corresponding to one of the plurality of unique labels.

In some embodiments, a plurality of barcoded cDNA sequences is produced in the chamber, each barcoded cDNA sequence of the plurality encoding a protein of interest, corresponding to any one of a plurality of different proteins, linked to a corresponding reverse complement barcode sequence. For example, barcoded cDNA sequences corresponding to up to 12 unique labels are pooled in single well. As barcoded cDNA sequences from specific exports can be identified based on a barcode sequence (~10 bp) on the capture object, without amplifying antibody transcripts from individual cells before TAP assembly, it may lead to the expression of non-clonal antibodies and make downstream characterization difficult. Accordingly, in some embodiments, the methods further comprise selectively amplifying the barcoded cDNA sequences to produce an amplified cDNA product (or further amplified cDNA product) encoding the protein of interest or a fragment thereof.

In some embodiments, the methods further comprise:
a. optionally amplifying a plurality of barcoded cDNA sequences;
b. selectively amplifying the plurality of barcoded cDNA sequences (or amplified cDNA sequences) using a barcode-specific forward primer and a reverse primer specific to the protein of interest to produce an amplified cDNA product (or further amplified cDNA product) encoding the protein of interest or a fragment thereof;
c. annealing a 5′ end of the amplified cDNA product (or further amplified cDNA product) to a 5′ corresponding end of a DNA fragment for transcriptionally-active PCR (TAP) to produce an annealed TAP product; and
d. amplifying the annealed TAP product via overlap extension PCR using a TAP adapter primer to produce a construct for expression of the protein of interest.

In some embodiments, the reverse primer specific to the protein of interest comprises a sequence complementary to a sequence encoding a conserved region (e.g., a constant portion) of the protein of interest, or a sequence 3′ to the conserved region (e.g., a 3′ UTR sequence). In some embodiments, a 3′ end of the amplified cDNA product (or further amplified cDNA product) comprises a region overlapping with a 3′ corresponding end of the DNA fragment for TAP.

In some embodiments, each barcoded cDNA sequence of the plurality encoding a heavy chain or a light chain sequence corresponding to any one of a plurality of different antibodies, linked to a corresponding reverse complement barcode sequence. In these embodiments, the method further comprising:
a. optionally amplifying the plurality of barcoded cDNA sequences;
b. selectively amplifying the plurality of barcoded cDNA sequences using a barcode-specific forward primer and a reverse primer targeting a conserved portion of the corresponding constant region sequence (e.g., a 5′ end, or sequence adjacent thereto, of the constant region) to produce an amplified cDNA product (or further amplified cDNA product) encoding the barcode-specific variable region;
c. annealing ends of the amplified cDNA product (or further amplified cDNA product) to corresponding ends of a DNA fragment for TAP to produce an annealed TAP product; and
d. amplifying the annealed TAP product via overlap extension PCR using TAP adapter primers to produce an expression construct for expression of an antibody heavy chain or light chain.

In some embodiments, amplifying the plurality of barcoded cDNA sequences comprises using a single primer (e.g., a P1 primer). In some embodiments, amplifying the plurality of barcoded cDNA sequences comprises using different forward and reverse primers.

In some embodiments, in step b of selectively amplifying, the barcode-specific forward primer may be a sequence comprising one of SEQ ID NO: 13-24. In some embodiments, in step b of selectively amplifying, the reverse primer targeting a conserved portion may be a sequence comprising SEQ ID NO: 54 or 55.

Further, provided herein are methods of preparing a construct for expression of the protein of interest.

In some embodiments, the methods comprise providing a barcoded cDNA sequence and the barcoded cDNA sequence comprises a nucleic acid encoding a protein of interest linked to the reverse complement of the barcode sequence of the first oligonucleotide. In some embodiments, the barcode cDNA sequence is produced by the methods described herein.

In some embodiments, the methods comprise amplifying at least a portion of the barcoded cDNA sequence using a barcode-specific primer and a primer specific to the nucleic acid encoding the protein of interest, thereby producing an amplified cDNA product.

In some embodiments, the methods comprise providing a DNA fragment for transcriptionally-active PCR (TAP) comprising:
i. a promoter sequence,
ii. a nucleic acid sequence complementary to a 5′ end of the nucleic acid encoding the protein of interest (e.g., 5′ end of the amplified cDNA product),
iii. a nucleic acid sequence complementary to a 3′ end of the nucleic acid encoding the protein of interest (e.g., a 3′ end of the amplified cDNA product), and
iv. a terminator sequence.

In some embodiments, the methods comprise incorporating the amplified cDNA product into the DNA fragment for TAP, thereby producing a construct for expression of the protein of interest.

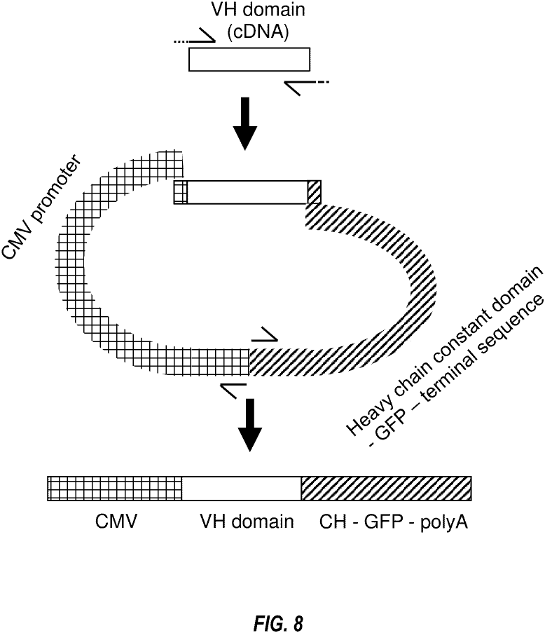

Transcriptionally-active PCR (TAP) as described in Clargo et al., mAbs 6:1, 143-159; January/February 2014 may be used to prepare a construct for antibody expression or, more generally, for expression of a protein complex. With TAP, an expression construct for a protein of interest (e.g., antibody heavy or light chain) can be directly generated without cloning genes into expression vectors or purifying fragments from the PCR reaction. In some embodiments, the transcriptionally-active PCRs (TAP) are used producing pairs of heavy and light chain variable domain genes as shown in FIG. 8, where a variable domain of a heavy chain of an antibody is amplified via PCR using a barcode-specific forward primer to bind to the barcode sequence at the 5′ end and a 3′ reverse primer targeting a conserved portion of the corresponding constant region sequence (e.g., a 5′ end, or sequence adj acent thereto, of the constant region) to produce an amplified cDNA product encoding the barcode-specific variable region (Vh). The amplified cDNA product includes overlap regions (~25 base pair) at the 5′ end overlapping with 3′ end of the promoter sequence (e.g., cytomegalovirus (CMV) promoter) and at the 3′ end with the 5′ end of a heavy or light chain constant domain sequence linked to a terminator sequence (such as a polyadenylation sequence). Then, the annealed TAP product is amplified via overlap extension PCR using TAP adapter primers to produce a linear TAP product, providing an expression construct for expression of an antibody heavy chain or light chain.

Similarly, a TAP product encoding a light chain of the antibody is generated via PCR reactions with the primers specific to the light chain variable domain. The pairs of separate TAP products, one encoding the heavy chain and the other encoding light chain, were subsequently used directly in transfection of cells and production of recombinant antibody.

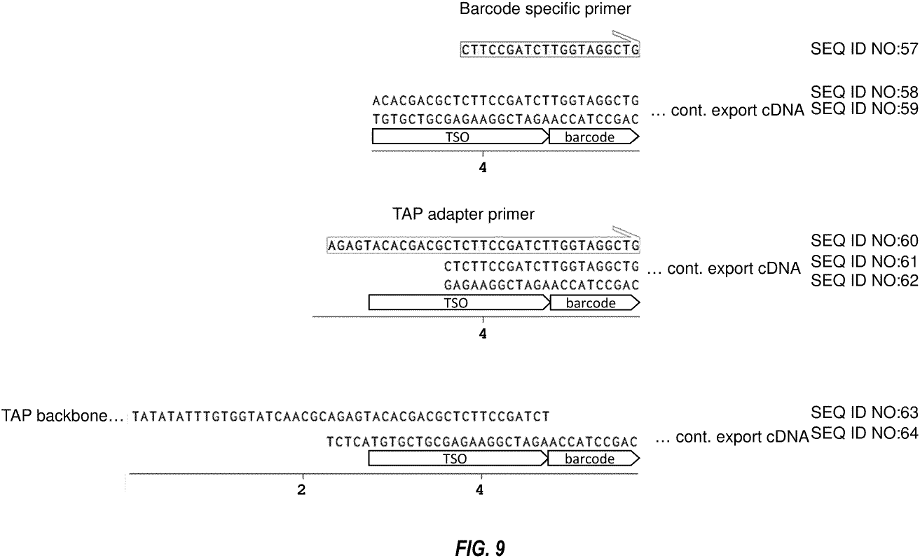

Accordingly, in some embodiments, the methods described herein are provided for preparing a construct for expression an antibody, or a fragment thereof, from the barcoded cDNA sequences, as shown in FIG. 9. In some embodiments, the methods of preparing a construct for antibody expression comprise:
a. providing a barcoded cDNA sequence produced by the method described herein, wherein the barcoded cDNA sequence comprises a nucleic acid encoding a heavy chain or a light chain of an antibody, or a fragment thereof, linked to the reverse complement of the barcode sequence of the first oligonucleotide;
b. amplifying at least a portion of the barcoded cDNA sequence using a barcode-specific primer and a primer specific to the nucleic acid encoding the heavy chain or the light chain of the antibody, thereby producing an amplified cDNA product;
c. providing a DNA fragment for transcriptionally active PCR (TAP), the DNA fragment comprising:
   i. a promoter sequence,
   ii. a nucleic acid sequence complementary to a 5′ end of the nucleic acid encoding the heavy chain or light chain sequence (e.g., 5′ end of the amplified cDNA product),
   iii. a nucleic acid sequence complementary to a 3′ end of the nucleic acid encoding the heavy chain or light chain sequence (e.g., a 3′ end of the amplified cDNA product),
   iv. a heavy or light chain constant domain sequence, and
   v. a terminator sequence;
d. incorporating the amplified cDNA product into the DNA fragment for TAP, thereby producing a construct for expression of the heavy chain or light chain of the antibody comprising a variable domain and a constant domain.

In some embodiments, the barcoded cDNA sequence comprises a nucleic acid encoding a heavy chain or a light chain variable domain of an antibody linked to a barcode sequence at a 5′ end.

In some embodiments, the amplified cDNA product comprises a heavy chain or light chain variable domain sequence.

In some embodiments, the DNA fragment for TAP comprises an antibody sequence encoding a heavy or light chain constant domain sequence 3′ to a respective variable domain.

In some embodiments, incorporating the amplified cDNA product into the DNA fragment for TAP comprises incorporating the amplified cDNA product encoding the variable region into the DNA fragment 3′ to the promoter sequence and 5′ to the sequence encoding the heavy or light chain constant domain sequence.

In some embodiments, the constant region sequence in the DNA fragment for TAP is a heavy chain constant region sequence. In some embodiments, wherein the heavy chain constant region sequence comprises one, two, or three tandem immunoglobulin domains. In some embodiments, the constant region sequence in the DNA fragment for TAP is a light chain constant region sequence.

In some embodiments, the promoter sequence comprises a cytomegalovirus (CMV) promoter sequence. In some embodiments, the promoter sequence provides constitutive gene expression. Any other known promoter suitable for constitutive gene expression may be used.

In some embodiments, the DNA fragment for TAP further comprises a sequence encoding fluorescent reporter protein. In some embodiments, the DNA fragment for TAP further a sequence encoding a self-cleaving peptide 5′ to the sequence encoding fluorescent reporter protein. In some embodiments, the self-cleaving peptide is T2A, P2A, E2A, or F2A. In some embodiments, the self-cleaving peptide is T2A.

In some embodiments, amplifying the barcoded cDNA sequence occurs by performing polymerase chain reaction (PCR) selective for barcoded cDNA sequences using the barcode-specific primer.

In some embodiments, incorporating the amplified barcoded cDNA sequence into the DNA fragment for TAP occurs by using overlap extension PCR. The overlap extension PCR generates overlap regions (~25 base pairs, for example) at the 5′ end with the promoter sequence and at the 3′ end with the constant domain sequence.

In some embodiments, the methods further comprise amplifying the expression construct.

In some embodiments, providing one or more barcoded cDNA sequence comprises providing a mixture of barcoded cDNA sequences, each barcoded cDNA sequence of the mixture encoding a heavy chain or a light chain sequence, corresponding to any one of a plurality of different antibodies, linked to a corresponding reverse complement barcode sequence.

In some embodiments, the methods described herein are provided for preparing producing a pair of expression constructs for the heavy chain and the light chain of an antibody from the barcoded cDNA sequences.

In some embodiments, the methods comprise providing a first barcoded cDNA sequence, comprising a nucleic acid encoding a heavy chain of an antibody, linked to a reverse complement of a first barcode sequence at a 5′ end; and providing a second barcoded cDNA sequence, comprising a nucleic acid encoding a light chain of the same antibody, linked to a reverse complement of a second barcode sequence at a 5′ end. In some embodiments, the first and second barcode sequences are the same. In some embodiments, the first and second barcode sequences are different.

In some embodiments, the methods comprise
a. providing a first DNA fragment for transcriptionally active PCR (TAP), the DNA fragment comprising
   i. a promoter sequence,
   ii. a constant domain sequence 3′ to a respective variable domain of the heavy chain, and
   iii. a terminator sequence;
b. providing a second DNA fragment for transcriptionally active PCR (TAP), the DNA fragment comprising:
   i. a promoter sequence,
   ii. a constant domain sequence 3′ to a respective variable domain of the light chain, and
   iii. a terminator sequence.

In some embodiments, the methods comprise
a. providing a first barcoded cDNA sequence, comprising a nucleic acid encoding a heavy chain of an antibody, linked to a first barcode sequence at a 5′ end;
b. providing a second barcoded cDNA sequence, comprising a nucleic acid encoding a light chain of the same antibody, linked to a second barcode sequence at a 5′ end;
c. amplifying at least a portion of the first barcoded cDNA sequence using a first barcode-specific primer;
d. amplifying at least a portion of the second barcoded cDNA sequence using a second barcode-specific primer;
e. providing a first DNA fragment for transcriptionally active PCR (TAP), the DNA fragment comprising:
   i. a promoter sequence,
   ii. a constant domain sequence 3′ to a respective variable domain of the heavy chain, and
   iii. a terminator sequence;
f. providing a second DNA fragment for transcriptionally active PCR (TAP), the DNA fragment comprising:
   i. a promoter sequence,
   ii. a constant domain sequence 3′ to a respective variable domain of the light chain, and
   iii. a terminator sequence;
g. incorporating the amplified cDNA products encoding the respective variable domain into the DNA fragment 3′ to the promoter sequence and 5′ to the corresponding constant domain sequence,
thereby producing a pair of expression constructs for the heavy chain and the light chain of an antibody.

### VII. Capture Objects

A capture object described herein may comprise a label, a plurality of first and second oligonucleotides. Each of first oligonucleotides includes a barcode sequence and a sequence comprising at least three consecutive guanine nucleotides at a 3′ end. Each of first oligonucleotides includes each second oligonucleotide of the plurality comprises a capture sequence.

Each of the first oligonucleotides of the plurality may include a 5′-most nucleotide and a 3′-most nucleotide, where the priming sequence may be adjacent to or comprise the 5′-most nucleotide, and where the barcode sequence may be located 3′ to the priming sequence and 5′ to the 3′-most nucleotide.

Each of the first oligonucleotides of the plurality may include a 5′-most nucleotide and a 3′-most nucleotide, the priming sequence may be adjacent to or comprise the 5′-most nucleotide, and where capture sequence may be adjacent to or comprise the 3′-most nucleotide.

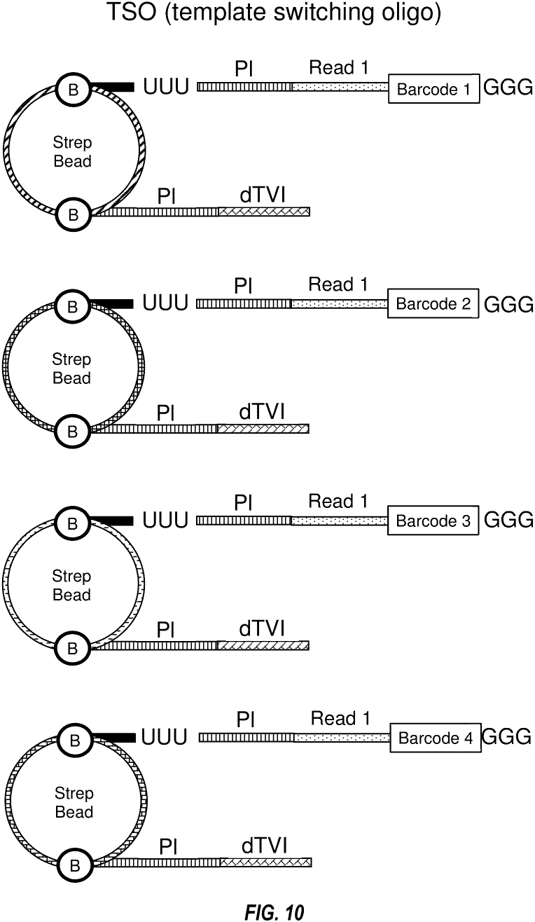

A schematic showing the construction of a plurality of capture objects is shown in FIG. 10. Each capture object has a bead to which first oligonucleotides and second oligonucleotides are attached, for illustrative purpose only one each of first oligonucleotide (top) and second oligonucleotide (bottom) is attached. The 5′ end of the first oligonucleotide, and in particular to the 5′ end of the first priming sequence is linked to the bead. The 5′ end of the second oligonucleotide, and in particular to the 5′ end of the second priming sequence, is attached to the capture object. Priming sequence (shown here as “P1”) are common to all oligonucleotides of all capture objects in this example, but in other embodiments, the linker and/or the priming sequence may be different for different oligonucleotides on a capture object or alternatively the linker and/or the priming sequence may be different for different capture objects in the plurality.

Priming sequence (shown here as “P1”) are common to all second oligonucleotides of all capture objects in this example, but in other embodiments, the linker and/or the priming sequence may be different for different second oligonucleotides on a capture object or alternatively the linker and/or the priming sequence may be different for different capture objects in the plurality.

Capture sequence of the second oligonucleotide is located at or proximal to the 3′ end of the second oligonucleotide. In this non-limiting example, the capture sequence is shown as a PolyT-VN sequence, which generically captures released RNA. In some embodiments, the capture sequence is common to all second oligonucleotides of all of the capture objects of the plurality of capture objects. However, in other pluralities of capture objects, the capture sequence on each second oligonucleotide of the capture object may not necessarily be the same.

Barcode sequence (~10 bp in length) of the first oligonucleotide is 3′ to the priming sequence. Each first oligonucleotide of the plurality on a single capture object has an identical barcode sequence, and the barcode sequence for the plurality of capture objects are different for each of the capture objects of the plurality.

In some embodiments, the ratio of the second oligonucleotide to the first oligonucleotide ranges from 1:10 to 10:1. In some embodiments, the ratio of the capture sequence of the second oligonucleotide to the first oligonucleotide sequence is about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1. In some embodiments, the ratio of the second oligonucleotide to the first oligonucleotide is about 1:1 (e.g., 95:100 to 100:95). The ratio can be measured by methods known in the field; in one nonlimiting example, two labeling molecules binding to the first oligonucleotide and the second oligonucleotide respectively can be introduced to the beads and the ratio can be determined by detecting the labeling molecules.

A plurality of capture objects. A plurality of capture objects is provided for use in multiplex nucleic acid capture. Each capture object of the plurality is a capture object according to any capture object described herein, wherein the barcode sequence of the first oligonucleotide of each capture object of the plurality is different from the barcode sequence of the first oligonucleotide of a capture object of the plurality having a different label. In some embodiments, the plurality of capture objects includes capture objects having at least 4 different types of barcodes (e.g., at least 12, 14, 16, 18, 20, 25, 30, 40, 50, 60, 70, 80, 90, 100, 150, 200, 250, 500, 1000, or more different barcodes). In some embodiments, the plurality comprises at least 4 types of capture objects, at least 8 types of capture objects, at least 12 types of capture objects.

In some embodiments, the plurality of capture objects may include at least 4 different types of capture objects (e.g., at least 12, 14, 16, 18, 20, 25, 30, 40, 50, 60, 70, 80, 90, 100, 150, 200, 250, 500, 1000, or more different capture object types). In other embodiments, the plurality of capture objects may include at least 10,000 capture objects.

### A. Template-switching Oligos (TSO)

During reverse transcription, upon reaching the 5′ end of the RNA, the terminal transferase activity of the reverse transcriptase adds a few additional nucleotides (usually starting with C, e.g., CCC). These additional nucleotides are used for priming the Template Switching Oligo (TSO) including at least three guanine nucleotides (e.g., GGG). In this template switching step, the reverse transcriptase switches from mRNA as a template to TSO as a template, as depicted in FIG. 7.

Accordingly, in some embodiments, the first oligonucleotide comprises at least three guanine nucleotides at a 3′ end. In some embodiments, the first oligonucleotide comprises 3, 4, 5, 6, 7, 8, or more guanine nucleotides at a 3′ end.

### B. Capture Sequence

The second oligonucleotide includes a capture sequence configured to capture RNA. The capture sequence is an oligonucleotide sequence having from about 6 to about 50 nucleotides. In some embodiments, the capture sequence captures RNA by hybridizing to RNA released from a cell of interest. One non-limiting example includes polyT sequences, (having about 30 to about 40 nucleotides) which can capture and hybridize to RNA fragments having Poly A at their 3′ ends. The polyT sequence may further contain two nucleotides VN or VI at its 3′ end. Other examples of capture sequences include random hexamers (“randomers”) which may be used in a mixture to hybridize to and thus capture complementary nucleic acids. Alternatively, complements to gene specific sequences may be used for targeted capture of nucleic acids, such as B cell receptor or T cell receptor sequences.

In various embodiments, the capture sequence of one or more (e.g., all or substantially all) of the plurality of second oligonucleotides may bind to one of the released RNA and primes the released RNA, thereby allowing a polymerase (e.g., reverse transcriptase) to transcribe the captured RNA.

In some embodiments, the capture sequence of the second oligonucleotide of the plurality of capture objects comprises an oligo-dT sequence. For example, the oligo-dT sequence may be a N(T)ₓVN sequence or an (T)ₓVI sequence, wherein X is greater than 10, 15, 20, 25, or 30.

In other embodiments, the capture sequence of one or more (e.g., each) of the plurality of second oligonucleotides may include a gene-specific primer sequence. In some embodiments, the gene-specific primer sequence may target (or may bind to) an mRNA sequence encoding a T cell receptor (TCR) (e.g., a TCR alpha chain or TCR beta chain, particularly a region of the mRNA encoding a variable region or a region of the mRNA located 3′ but proximal to the variable region). In other embodiments, the gene-specific primer sequence may target (or may bind to) an mRNA sequence encoding a B-cell receptor (BCR) (e.g., a BCR light chain or BCR heavy chain, particularly a region of the mRNA encoding a variable region or a region of the mRNA located 3′ but proximal to the variable region).

### C. Priming and Other/additional Sequences

The oligonucleotide of the capture object has a priming sequence, and the priming sequence may be adjacent to or comprises the 5′-most nucleotide of the oligonucleotide(s). The priming sequence may bind to a primer that, upon binding, primes a reverse transcriptase.

In some embodiments, the first oligonucleotide comprises a first priming sequence that corresponds to a first primer sequence and/or wherein the second oligonucleotide comprises a second priming sequence that corresponds to a second primer sequence. In some embodiments, the first and second primer sequences are the same.

The priming sequence may be a generic or a sequence-specific priming sequence.

In some embodiments, the generic priming sequence may correspond to a P1 primer, a P5 primer, or a P7 primer. primer. In some embodiments, the priming sequence of the oligonucleotides described herein may be a sequence comprising one of SEQ ID NOs: 50-53.

In some embodiments, the first oligonucleotide comprises one or more uridine nucleotides 5′ to the barcode sequence and, if present, the first priming sequence. In some embodiments, the first oligonucleotide comprises three uridine nucleotides 5′ to the barcode sequence and, if present, the first priming sequence. In some embodiments, the one or more uridine nucleotides are adjacent to or comprise the 5′-most nucleotide(s) of the first oligonucleotide.

### D. Modifications

The first and/or the second oligonucleotides contained on the capture objects, as described herein, may include modifications. These modifications may afford a wide range of tunable functionality for the first and second oligonucleotides. A modification of the first or second oligonucleotide may include non-natural nucleotide moieties or other small organic molecular moieties which provide for stable connection to a capture object as known in the art. Exemplary modifications include but are not limited to an amine-modified oligonucleotide; thiol-modified oligonucleotide, disulfide- modified oligonucleotide, hydrazide-modified succinate-modified oligonucleotide, or proprietary linker-modified oligonucleotide (commercially available or otherwise) which may be present at the 5′ or 3′ terminus of the first and/or second oligonucleotides, depending on the selected usage. Alternatively, the first and/or the second oligonucleotide may include a biotin, streptavidin, or other biomolecule capable of binding to a respective binding molecule on the capture object. Further the first and/or the second oligonucleotide may include an azidyl-modification or alkynyl-modification, permitting Click coupling to a reaction pair moiety on the capture object. Other modifications may include other non-nucleotide containing moieties, proximal to such terminal modifications to reduce steric interference for priming sequences, capturing sequences, barcoding sequences, labelling sequences, or any other sequence module of the first and second oligonucleotides.

The first and/or the second oligonucleotide may include, within the respective nucleotide sequences, one or more modified nucleotide moieties which may improve the stability of the first and/or the second oligonucleotide to conditions used throughout the methods as described herein. The modifications may increase stability of the first and/or the second oligonucleotide with respect to one or more of melting temperature, affinity for a target nucleotide, resistance to a nuclease, and the like. In some alternative embodiments, modified first and/or second oligonucleotides may provide for enhanced susceptibility to one or more nucleases or selective chemical, photochemical and/or thermal cleavages along its length.

The first and/or second oligonucleotide can have various nucleic acid residues, such as for example, an unmodified nucleotide moiety, a modified nucleotide moiety, or any other feature as long as the polymerizing agent is capable of functioning on the primer as a viable substrate.

The first and/or second oligonucleotide may include one or more modified nucleotides capable of incorporation into a primer in the place of a ribosyl or deoxyribosyl moiety. The modified nucleotides may be modified at the 2′ position of sugar moiety of the nucleoside, which may include substituted, unsubstituted, saturated, unsaturated, aromatic or non-aromatic moieties. Suitable moieties at the 2′ position include, but are not limited to, alkoxy (such as methoxy, ethoxy, propoxy), 2′-oxy-3-deoxy, 2′-t-butyldimethylsilyloxy, furanyl, propyl, pyranosyl, pyrene, acyclic moieties, and the like. In other embodiments, a 2′ modification may include a 2′ fluoro- modified nucleotide, a 2′ alkoxyalkyl (e.g., 2′O- methoxyethyl (MOE), or the like. Further the modified nucleotide may be a locked nucleic acid (LNA), an unlocked nucleic acid or an unnatural nucleotide analog such as, but not limited to, 5-nitroindole, 5-methyl dC, Super T® (IDT), Super G ® (IDT) and the like.

### E. Other Features of Capture Objects

A capture object may be of any suitable size, as long as it is small enough to pass through the flow channel(s) of the flow region and into/out of a sequestration pen of the microfluidic device with which it is being used, e.g., any microfluidic device as described herein. Further, the capture object may be selected to have a sufficiently large number of oligonucleotides linked thereto, such that nucleic acid may be captured in sufficient quantity to generate a nucleic acid library useful for sequencing. In various embodiments, the capture object may be a bead. For example, the capture object can be a bead (or similar object) having a core that includes a paramagnetic material, a polymeric material and/or glass. The polymeric material may be polystyrene or any other plastic material which may be functionalized to link the plurality of oligonucleotides. In some embodiments, the capture object may be a spherical or partially spherical bead and have a diameter greater than about 5 microns and less than about 40 microns. In some embodiments, the spherical or partially spherical bead may have a diameter of about 5, about 7, about 8, about 10, about 12, about 14, about 16, about 18, about 20, about 22, about 24, or about 26 microns, or any range defined by two of the foregoing values.

In some embodiments, the capture object has a composition such that it is amenable to movement using a dielectrophoretic (DEP) force, such as a negative DEP force. For example, the capture object can be a bead (or similar object) having a core that includes a paramagnetic material, a polymeric material and/or glass. The polymeric material may be polystyrene or any other plastic material which may be functionalized to link the oligonucleotides. The core material of the capture object may be coated to provide a suitable material to attach linkers to the oligonucleotides, which may include functionalized polymers, although other arrangements are possible. The linkers used to link the oligonucleotides to the capture object may be any suitable linker as is known in the art. The linker may include hydrocarbon chains, which may be unsubstituted or substituted, or interrupted or non-interrupted with functional groups such as amide, ether or keto- groups, which may provide desirable physicochemical properties. The linker may have sufficient length to permit access by processing enzymes to priming sites near the end of the oligonucleotide linked to the linker. The oligonucleotides may be linked to the linker covalently or non-covalently, as is known in the art. A nonlimiting example of a non-covalent linkage to the linker may be via a biotin/streptavidin pair.

In some embodiments, the first oligonucleotide is linked to the capture object. In some embodiments, the first oligonucleotide is covalently bound to the capture object. In some embodiments, the first oligonucleotide is linked to the capture object by streptavidin-biotin binding.

In some embodiments, the second oligonucleotide is linked to the capture object. In some embodiments, the second oligonucleotide is covalently bound to the capture object. In some embodiments, the second oligonucleotide is linked to the capture object by streptavidin-biotin binding.

Additional priming and/or adaptor sequences. The second oligonucleotide(s) (sometimes referred herein as “capture oligonucleotide”) may optionally have one or more additional priming/adaptor sequences, which either provide a landing site for primer extension or a site for immobilization to complementary hybridizing anchor sites within a massively parallel sequencing array or flow cell.

Optional oligonucleotide sequences. Each capture oligonucleotide of the plurality of capture oligonucleotides may optionally further include a unique molecule identifier (UMI) sequence. Each capture oligonucleotide of the plurality may have a different UMI from the other capture oligonucleotides of a capture object, permitting identification of unique captures as opposed to numbers of amplified sequences. In some embodiments, the UMI may be located 3′ to the priming sequence and 5′ to the capture sequence. The UMI sequence may be an oligonucleotide having about 5 to about 20 nucleotides. In some embodiments, the oligonucleotide sequence of the UMI sequence may have about 10 nucleotides.

In some embodiments, each capture oligonucleotide of the plurality of capture oligonucleotides may also include a Not1 restriction site sequence (GCGGCCGC, SEQ ID NO: 56). The Not1 restriction site sequence may be located 5′ to the capture sequence of the capture oligonucleotide. In some embodiments, the Not1 restriction site sequence may be located 3′ to the barcode sequence of the capture oligonucleotide.

In other embodiments, each capture oligonucleotide of the plurality of capture oligonucleotides may also include additional indicia such as a pool Index sequence. The Index sequence is a sequence of 4 to 10 oligonucleotides which uniquely identify a set of capture objects belonging to one experiment, permitting multiplex sequencing combining sequencing libraries from several different experiments to save on sequencing run cost and time, while still permitting deconvolution of the sequencing data, and correlation back to the correct experiment and capture objects associated therein.

### F. Exemplary Barcode Sequences, First and Second Oligonucleotides

Set of barcode sequences. In various embodiments, the method may further include: selecting each barcode sequence from a set of 12 to 100 non-identical oligonucleotide sequences. In some embodiments, the set of barcode sequences may consist essentially of 2, 3, 4, 5, 6, 7, 8, 9, 10, 11, or 12 barcode sequences.

An exemplary set of barcode sequences is provided in Table 8, which includes 12 non-identical barcode sequences (SEQ ID NOs: 1-12), each barcode sequence of the set having a structure according to any barcode as described herein. Examples of corresponding barcode-specific forward primers are provided in Table 8 (as SEQ ID NOs: 13-24). Examples of corresponding demultiplexing forward primers are provided in Table 8 (as SEQ ID NOs: 25-36).

Some exemplary, but not limiting first oligonucleotides are illustrated in Table 8. In some embodiments, the first oligonucleotides including a first priming sequence, a barcode sequence, and optionally UUU at the 5′ end and at least three guanine nucleotides at the 3′ end may be a sequence comprising one of SEQ ID NOs: 37-48.

Some exemplary, but not limiting second oligonucleotides are illustrated in Table 8. In some embodiments, the second oligonucleotide comprising a second priming sequence and a capture sequence may be a sequence comprising /5Biosg/AAGCAGTGGTATCAACGCAGAGTACTTTTTTTTTTTTTTTTTTTTTTTTTTTTTTVI (SEQ ID NO: 49).

### VIII. Methods of Assembling Full Length V(D)J Sequences From Fragmented NGS (Next Generation Sequencing, Massively Parallel Sequencing) Data

In another aspect, methods are provided to assemble complete V(D)J sequences from fragmented NGS data originating from a single antibody producing cell (e.g., a B-Cell). Antibody producing cells (e.g.,B-Cells) are expected to have one heavy chain and one light chain sequence that together form an antibody. The V(D)J region of the heavy and light chains is also known as the variable region and represent the part of an antibody responsible for binding to a specific antigen.

In some cases, antibody producing cells are known to have more than one heavy and light chain. Additionally, there is always the possibility that single cell NGS data might be contaminated, and an algorithm is needed that can identify all unique variable region sequences in a sample.

The variable region of heavy chains contains a V, D, and J region in that order. The variable region of light chains contains a V and J region in that order. Unlike heavy chains there are 2 types of light chains, Kappa and Lambda. When light chains are formed typically only one type of light chain is retained by the cell, and the V and J will be from the same type of light chain (i.e., V and J from Kappa or Lambda, but no mixing of V and J alleles between Kappa and Lambda). Each of these genes have many possible alleles and all versions of those alleles are well characterized.

When V, D, and J alleles are combined to form a given chain it is not uncommon for them to have inconsistent recombination sites causing the appearance of deletions with regards to the reference alleles or insertions between the alleles in the final assembly. Additionally, antibody producing cells (e.g., B-Cells) can go through a phase of Somatic Hypermutation creating mismatches with respect to the reference alleles. These variations are of critical importance to the function of the antibody that is produced. Therefore, it may not be sufficient to simply identify the reference alleles that the final sequence is constructed from, and the real sequence should be identified with all of its variation relative to the reference.

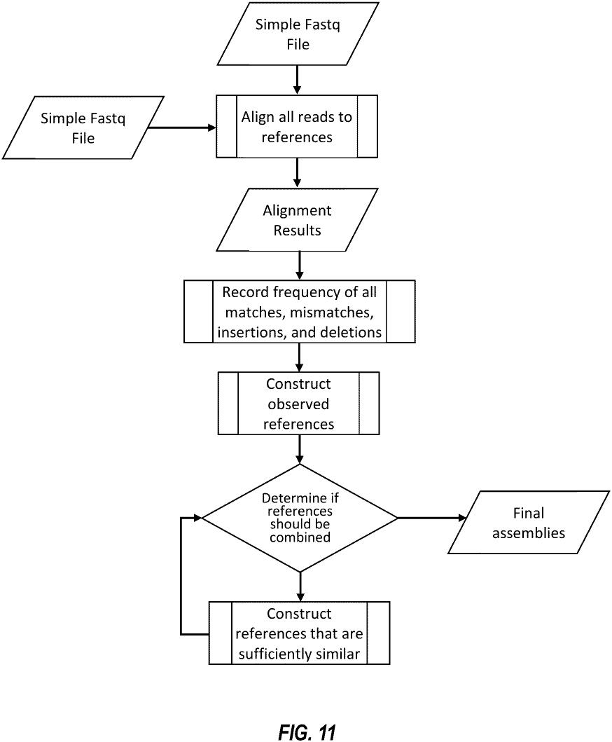

Accordingly, an assembly algorithm is provided to identify and correlate the correct sections of sequence fragment, and the overall approach is shown schematically in FIG. 11.

Reference-Based Assembly. Many steps in the assembly algorithm may include a reference-based assembly. The reference-based assembly performs sequence assemblies by aligning the sequence reads obtained by massively parallel sequencing techniques with a reference sequence. The massively parallel sequencing may be a 75×75 or a 150×150 sequencing experiment. Speed and the accuracy of sequence assembly can be improved using the reference-based assembly methods described herein, and computing demand can be decreased. The reference-based assembly may be conducted as follows:

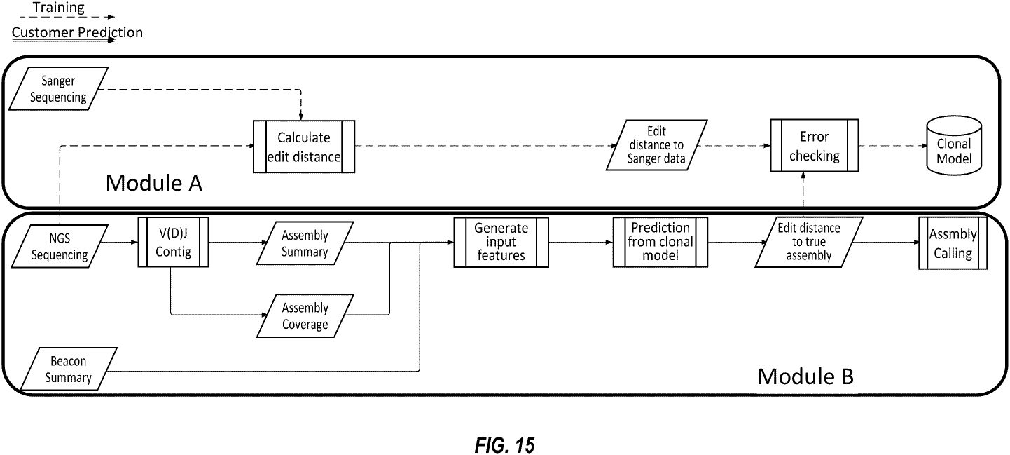

All reads are aligned from the sample to a set of references. The reference set may be provided as described below, and shown schematically in FIG. 15.

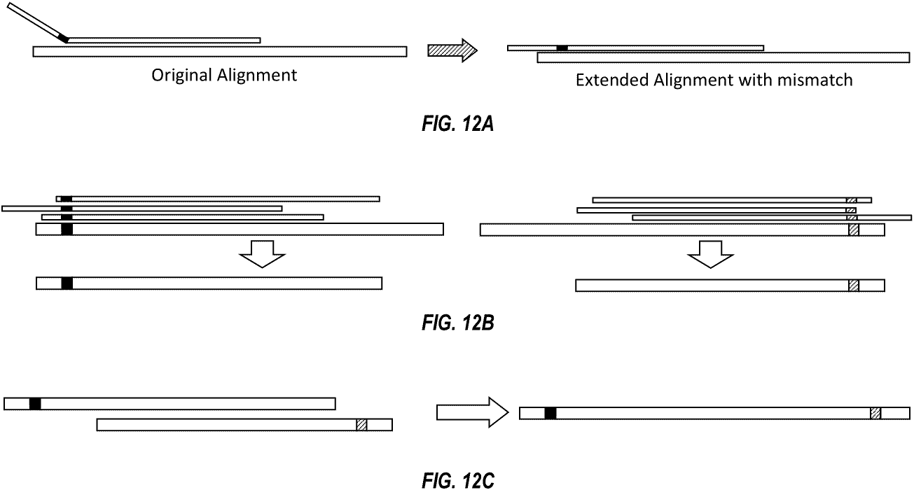

All aligned reads are reviewed and the frequency of each type of base that aligned to each base of the reference is recorded, as well as the frequency and types of insertions and deletions relative to the reference. Alignment algorithms may have difficulty aligning reads to a reference sequence when the reads have mismatches close to the start or end of the reference. When this can be identified, the alignment may be extended to the end of the reference to capture the mismatch. If a read aligns to a reference with the aligned portion of the read starting or ending close to the start or end of the reference and the aligned read has unaligned base pairs that overhang the start or end of the reference, the alignment can be extended to the end of the reference sequence, as shown in FIG. 12A.

A new sequence may be constructed for each reference in the original set by going through each nucleotide of the sequence and adding the most frequently occurring base from the alignment data. The new sequences may be modified based on the insertions and deletions recorded from alignment. To include an insertion, it will preferably occur at least half as frequently as the base before and after the insertion. To include a deletion, it will preferably occur more frequently than any of the bases it is deleting.

Partial references can be constructed if aligned reads do not cover the entire sequence. The example shown in FIG. 12B has two reference sequences that are very similar and have reads that align without mismatches, insertions, or deletions.

Consensus sequences may be built from references that can be combined due to high degree of similarity, as shown in FIG. 12C.

All final sequences that have more than 0.5% of total sample reads supporting the reference sequence may be reported.

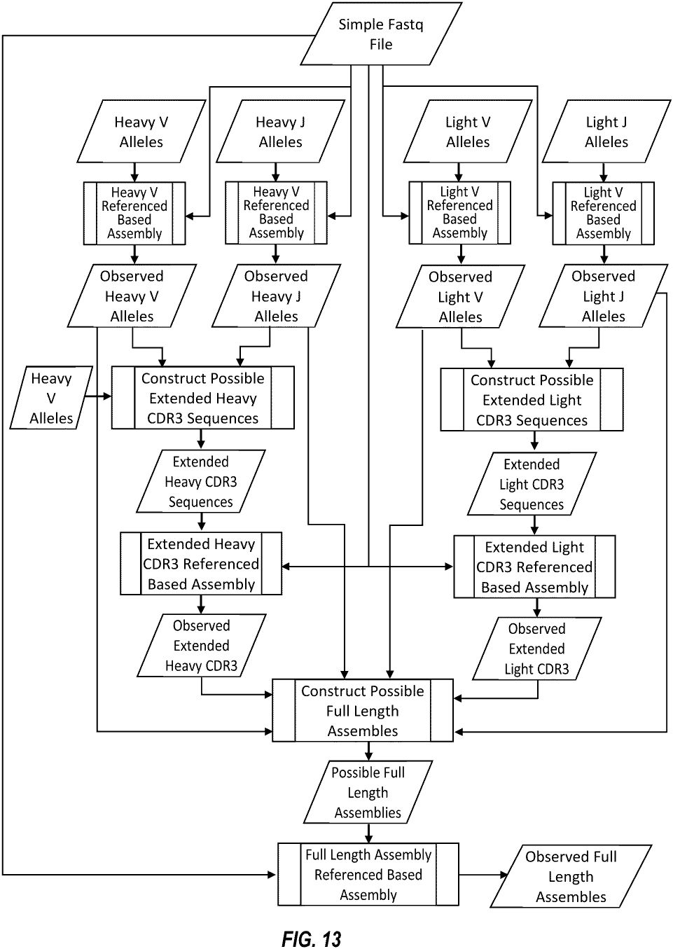

Method of assembling sequences using the reference-based algorithms. FIG. 13 shows how the reference-based assembly of each segment of heavy and light chains may be incorporated into the overall assembly algorithm. In some embodiments, the segments may be assembled from either 75×75 or 150×150 sequence fragments obtained from massively parallel (NGS) sequencing experiments.

The observed V and J sequences for heavy and light chains are identified. This may be performed by performing reference-based assembly on the following reference sets, which may be obtained from the IMGT database (International ImMunoGeneTics information system for immunoglobulins or antbodies): Heavy V alleles, Heavy J alleles, Light V alleles, Light J alleles,

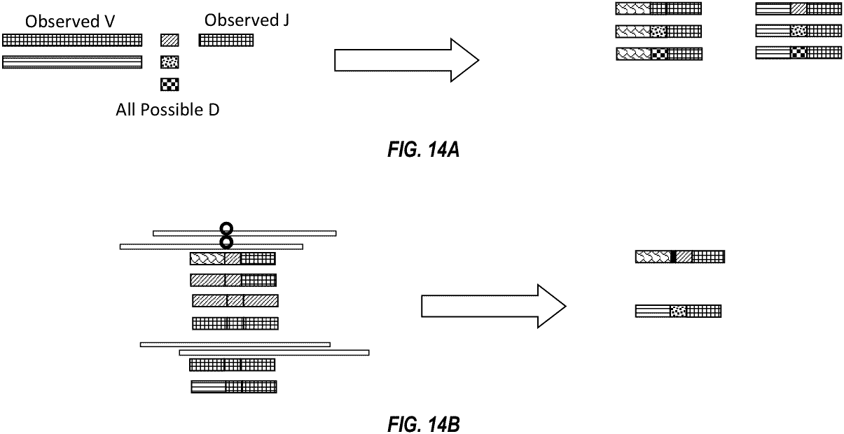

The observed set of “Extended Heavy CDR3 regions” are identified. This may be performed by the following operations: The terminal base pairs (e.g., the last 10, 15, 25, 30, 35, 40, 45, 50, 55, 60 or more base pairs) of all observed Heavy V alleles may be extracted to create a set of Heavy V ends. The initial base pairs (e.g., the first 10, 15, 25, 30, 35, 40, 45, 50, 55, 60 or more base pairs) of all observed Heavy J alleles may be extracted to create a set of Heavy J starts. If the Heavy J allele has fewer than the pre-selected initial base pairs (e.g., 40 bases), the entire sequence may be used to create the set. All known Heavy D alleles are obtained. All possible combinations of Heavy V ends, Heavy D alleles, and Heavy J starts may be constructed, in that order, to create an “Extended Heavy CDR3” reference set, as shown in FIG. 14A.

Reference-based assembly may be performed on this new set to find the observed “Extended Heavy CDR3s”. In the example shown in FIG. 14B, there is an observed sequence between one of the V and D alleles.

The observed set of “Extended Light CDR3 regions” may then be identified. This may be accomplished by the following operations. The terminal base pairs (e.g., the last 10, 25, 30, 35, 40, 45, 50, 55, 60 or more base pairs) of all observed Light V alleles may be extracted to create a set of Light V ends. The initial base pairs (e.g., the first 10, 25, 30, 35, 40, 45, 50, 55, 60 or more base pairs) of all observed Light J alleles may be extracted to create a set of Light J starts. If the Light J allele has fewer than the pre-selected initial base pairs (e.g., 40 bases), take the entire sequence. All possible combinations of Light V ends and Light J starts may be constructed, in that order, to create an “Extended Light CDR3” reference set. Reference-based assembly may be performed on this new set to find the observed “Extended Light CDR3s”

The observed full length variable sequences may then be identified, by the following operations:

Possible full length heavy chain references may be constructed for all observed “Extended Heavy CDR3s”, by:
a. Identifying the observed Heavy V allele having a terminus that most strongly overlaps with the start of the “Extended Heavy CDR3”.
b. Identifying the observed Heavy J allele having a terminus that most strongly overlaps with the end of the “Extended Heavy CDR3”.
c. Constructing possible full length heavy chain variable sequences by using the observed Heavy V allele, the observed Heavy J allele, and the observed extended heavy CDR3 according to the overlapping sequences, giving preference to the CDR3 when resolving mismatches or indels.

Possible full length light chain references may be constructed by the following operations:
a. Identifying the observed Light V allele having a terminus that most strongly overlaps with the start of the “Extended Light CDR3”.
b. Identifying the observed Light J allele having a terminus that most strongly overlaps with the end of the “Extended Light CDR3”.
c. Constructing possible full length light chain variable sequences by using the observed Light V allele, the observed Light J allele, and the observed extended light CDR3 according to the overlapping sequences, giving preference to the CDR3 when resolving mismatches or indels.

A combined reference set may then be created.

Perform reference-based assembly may then be performed to find the observed full length variable sequences. This final reference-based assembly also fixes any possible errors in constructing the reference sequences.

### IX Sanger Sequencing Based Reference

Sanger sequencing results were utilized to train a machine learning algorithm to identify sequences originating from individual pens using NGS sequence results. As shown in FIG. 15, Module A is used to develop a clonal model for experiment. In the operations of the training portion of the algorithm, comparisons including up to 140 features were used to develop the clonal model (e.g., full model (merged model + nullable). While 135-140 features provided excellent accuracy and precision, acceptable accuracy was obtained using as few as 30 features selected from the full set. A compact set of features, having 50 features, provides accuracy and precision meeting, and even exceeding, the accuracy and precision found with the model using 135 features.

Table 1 contains a list of features having a feature importance of greater than 0.008 for the full model (merged model + nullable).

TABLE 1

| No. | Feature | Importance | No. | Feature | Importance |
|---|---|---|---|---|---|
| 1 | percent_assembly reads | 0.13488676 | 16 | cov_113 | 0.01228165 |
| 2 | barcode_color | 0.09732204 | 17 | cov_37 | 0.01140315 |
| 3 | totalChain | 0.05011147 | 18 | cov_87 | 0.01081742 |
| 4 | cov_108 | 0.03178951 | 19 | cov_117 | 0.01079252 |
| 5 | cov_1 | 0.02741755 | 20 | cov_0 | 0.01060491 |
| 6 | total_heavy | 0.02420187 | 21 | cov_50 | 0.01022766 |
| 7 | cov_109 | 0.02208113 | 22 | cov_106 | 0.01001359 |
| 8 | nt_length | 0.01992039 | 23 | cov_107 | 0.00999279 |
| 9 | chimera_score | 0.01975148 | 24 | cov_48 | 0.00998143 |
| 10 | total_light | 0.01974904 | 25 | NumCellsUnpenned | 0.00978322 |
| 11 | cdr2_aa_length | 0.01874339 | 26 | cov_118 | 0.00899648 |
| 12 | chain_type | 0.01513904 | 27 | cov_97 | 0.00834949 |
| 13 | cov_88 | 0.01501483 | 28 | cov_60 | 0.00825807 |
| 14 | cov_51 | 0.01470301 | 29 | cov_119 | 0.00808127 |
| 15 | cdr3_aa_length | 0.01387441 | | | |

Table 2 contains a list of features having a feature importance of less than 0.0008 for the full model (merged model + nullable)

TABLE 2

| No. | Feature | Importance | No. | Feature | Importance |
|---|---|---|---|---|---|
| 30 | cov_112 | 0.00793725 | 83 | cov_7 | 0.00267119 |
| 31 | UnverifiedUnpen Success | 0.00749018 | 84 | cov_28 | 0.00263476 |
| 32 | cov_49 | 0.00717805 | 85 | cov_56 | 0.00259653 |
| 33 | cov_53 | 0.00706491 | 86 | cov_46 | 0.00256055 |
| 34 | cov_59 | 0.00687551 | 87 | cov_6 | 0.00249187 |
| 35 | cov_99 | 0.00680053 | 88 | cov_82 | 0.00242178 |
| 36 | cov_93 | 0.00659014 | 89 | cov_5 | 0.00238974 |
| 37 | cov_98 | 0.00645001 | 90 | cov_11 | 0.00236259 |
| 38 | cov_61 | 0.00641327 | 91 | cov_83 | 0.00236018 |
| 39 | cdr1_aa_length | 0.00636796 | 92 | cov_3 | 0.00233374 |
| 40 | cov_58 | 0.00635875 | 93 | cov_31 | 0.00233178 |
| 41 | cov_100 | 0.00614383 | 94 | cov_13 | 0.00232144 |
| 42 | chain_index | 0.00589706 | 95 | cov_4 | 0.00227171 |
| 43 | cov_25 | 0.00570587 | 96 | cov_81 | 0.00224191 |
| 44 | uniformity95_5 | 0.00566731 | 97 | cov_71 | 0.00222566 |
| 45 | cov_47 | 0.00554503 | 98 | cov_15 | 0.00219491 |
| 46 | cov_17 | 0.00552746 | 99 | cov_54 | 0.00202219 |
| 47 | cov_111 | 0.00546038 | 100 | cov_57 | 0.00198246 |
| 48 | cov_110 | 0.0053381 | 101 | cov_24 | 0.00180943 |
| 49 | cov_16 | 0.00533135 | 102 | cov_19 | 0.00180237 |
| 50 | cov_101 | 0.00531684 | 103 | cov_14 | 0.00175177 |
| 51 | cov_90 | 0.00523051 | 104 | cov_96 | 0.00169785 |
| 52 | cov_29 | 0.00517171 | 105 | cov_44 | 0.00167004 |
| 53 | cov_103 | 0.00502226 | 106 | cov_21 | 0.00166661 |
| 54 | cov_35 | 0.00491194 | 107 | cov_33 | 0.00163053 |
| 55 | cov_36 | 0.00490216 | 108 | cov_79 | 0.00162344 |
| 56 | cov_9 | 0.00453705 | 109 | cov_77 | 0.00161934 |
| 57 | cov_114 | 0.00436248 | 110 | cov_34 | 0.00157118 |
| 58 | cov_116 | 0.00430934 | 111 | cov_22 | 0.0015238 |
| 59 | cov_104 | 0.0042527 | 112 | cov_39 | 0.00143602 |
| 60 | cov_95 | 0.00383903 | 113 | cov_64 | 0.00143543 |
| 61 | cov_73 | 0.00380893 | 114 | cov_12 | 0.00140005 |
| 62 | cov_26 | 0.00377299 | 115 | cov_8 | 0.00139841 |
| 63 | cov_115 | 0.00374886 | 116 | cov_89 | 0.00134276 |
| 64 | cov_91 | 0.00368684 | 117 | cov_52 | 0.00130813 |
| 65 | cov_102 | 0.00367221 | 118 | cov_75 | 0.00130418 |
| 66 | cov_105 | 0.00364628 | 119 | cov_27 | 0.00125696 |
| 67 | cov_74 | 0.00362533 | 120 | cov_80 | 0.00120217 |
| 68 | cov_86 | 0.00350795 | 121 | cov_70 | 0.00113529 |
| 69 | cov_85 | 0.00343147 | 122 | cov_30 | 0.001073 |
| 70 | cov_40 | 0.00337501 | 123 | cov_23 | 0.0010349 |
| 71 | cov_18 | 0.00322563 | 124 | cov_43 | 0.00100188 |
| 72 | cov_38 | 0.00322112 | 125 | cov_78 | 0.00087833 |
| 73 | cov_10 | 0.00313785 | 126 | cov_76 | 0.00085684 |
| 74 | cov_2 | 0.00313284 | 127 | cov_69 | 0.00085654 |
| 75 | cov_32 | 0.00309601 | 128 | cov_68 | 0.00082919 |
| 76 | cov_45 | 0.00308953 | 129 | cov_41 | 0.00079478 |
| 77 | cov_94 | 0.00302882 | 130 | cov_65 | 0.00078142 |
| 78 | cov_55 | 0.00287788 | 131 | cov_42 | 0.00071942 |
| 79 | cov_84 | 0.00285239 | 132 | cov_63 | 0.0007127 |
| 80 | cov_72 | 0.00283859 | 133 | cov_66 | 0.00065801 |
| 81 | cov_92 | 0.00282351 | 134 | cov_20 | 0.00063121 |
| 82 | cov_62 | 0.0026922 | 135 | cov_67 | 0.00059034 |

In Table 3, a set of features is shown for the compact set.

TABLE 3

| No. | Feature | Importance | No. | Feature | Importance |
|---|---|---|---|---|---|
| 1 | percent_assembly reads | 0.14538409 | 26 | cov_48 | 0.01202331 |
| 2 | barcode_color | 0.13838243 | 27 | cov_53 | 0.010517 |
| 3 | totalChain | 0.06649052 | 28 | cov_106 | 0.00989941 |
| 4 | chain_type | 0.05593161 | 29 | cov_49 | 0.00911674 |
| 5 | cov_108 | 0.03687575 | 30 | cov_93 | 0.00873677 |
| 6 | cov_1 | 0.03648595 | 31 | cov_112 | 0.0084324 |
| 7 | cov_109 | 0.03464852 | 32 | cov_100 | 0.00836917 |
| 8 | cov_0 | 0.02953576 | 33 | cov_99 | 0.00802632 |
| 9 | cov_51 | 0.02567839 | 34 | cov_17 | 0.00788566 |
| 10 | cov_25 | 0.02561054 | 35 | cov_101 | 0.00668119 |
| 11 | cov_37 | 0.02497421 | 36 | cov_58 | 0.00607603 |
| 12 | total_light | 0.02326205 | 37 | cdr1_aa_length | 0.00514594 |
| 13 | cdr3_aa_length | 0.01941174 | 38 | cov_118 | 0.00503291 |
| 14 | cov_88 | 0.01816266 | 39 | cov_97 | 0.00446025 |
| 15 | chain_index | 0.01768483 | 40 | cov_60 | 0.00437193 |
| 16 | nt _length | 0.01727029 | 41 | cov_61 | 0.00432937 |
| 17 | cov_107 | 0.01682552 | 42 | cov_111 | 0.00424489 |
| 18 | cdr2_aa_length | 0.01680029 | 43 | cov_59 | 0.00401305 |
| 19 | UnverifiedUnpen Success | 0.016726 | 44 | NumCellsUnpenned | 0.00398344 |
| 20 | cov_50 | 0.01621451 | 45 | uniformity95_5 | 0.00366323 |
| 21 | chimera_score | 0.01477338 | 46 | cov_119 | 0.0034652 |
| 22 | total_heavy | 0.01457163 | 47 | cov_47 | 0.00308524 |
| 23 | cov_110 | 0.01324299 | 48 | cov_16 | 0.003004 |
| 24 | cov_87 | 0.01273379 | 49 | cov_117 | 0.0026794 |
| 25 | cov_113 | 0.01247092 | 50 | cov_98 | 0.00260878 |

Accuracy. Using the full set of 135 features, an accuracy of 83% and an FI score of 87% was obtained. Three sets of data (size equal to 284, 285, 284 respectively) were analyzed using the compact model of 50 features as shown in Table 6, as well as tolerating some null column values. A respective accuracy of 89% (F1-score of 92%); accuracy of 93% (F1-score 96%); and accuracy 91% (F1 score 94%) were obtained, showing excellent, even improved performance for the compact model.

Representative description of features is as shown in Table 4.

TABLE 4

| | |
|---|---|
| Percent _assembly_reads | Percent of reads of a sample aligned to the assembly vs all reads for that sample |
| Barcode_color | Color barcode (CFTD code) |
| totalChain | Total number of assemblies of its chain type (H or L) in a sample |
| total_heavy | Total number of assemblies of heavy chains |
| nt-length | Length of ig vdj nucleotide |
| chimera_score | The maximum coverage^{b} divided by the maximum coverage drop^{d} over an ig assembly |
| total_light | Total number of assemblies of light chains in a sample |
| cdr1_aa_length | The length of cdr1 amino acid of the assembly |
| Cdr2_aa_length | The length of cdr2 amino acid of the assembly |
| Cdr3_aa_length | The length of cdr3 amino acid of the assembly |
| chain_type | Heavy or Light chain of the assembly |
| NumCellsUnpenned | Number of unpenned cells reported by instrument algorithm |
| UnverifiedUnpenSuccess | A Boolean value that indicates whether the ‘NumCellsUnpenned’ is non-zero |
| Chain_index | The Numeric index of assemblies of a certain chain type (H or L) ordered by descending Percent_assembly_reads, starting at 1 |
| Uniformity95_5 | The 95 percentile of uniformity scores^{a} over the 5 percentile of uniformity scores^{a} |
| Cov_108 | The average of the coverage scores of vdj loci 325, 326 and 327 |

### X. Microfluidic Device and System

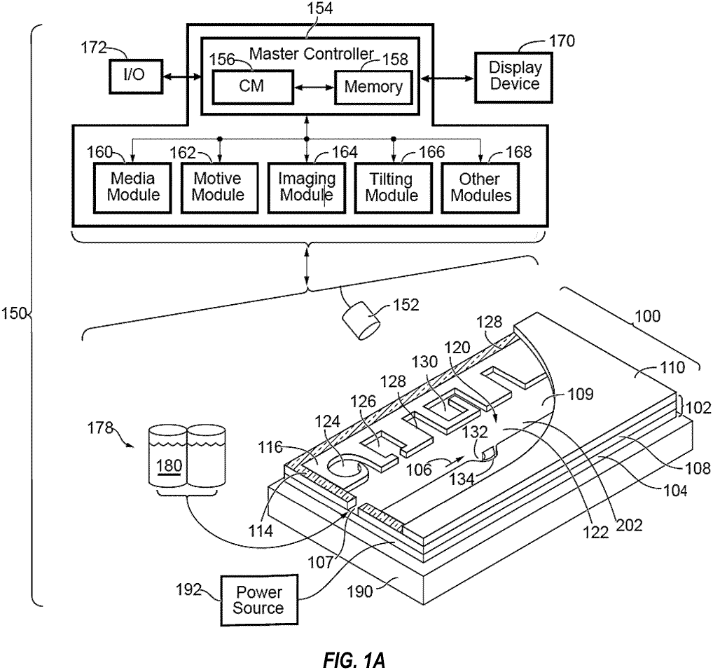

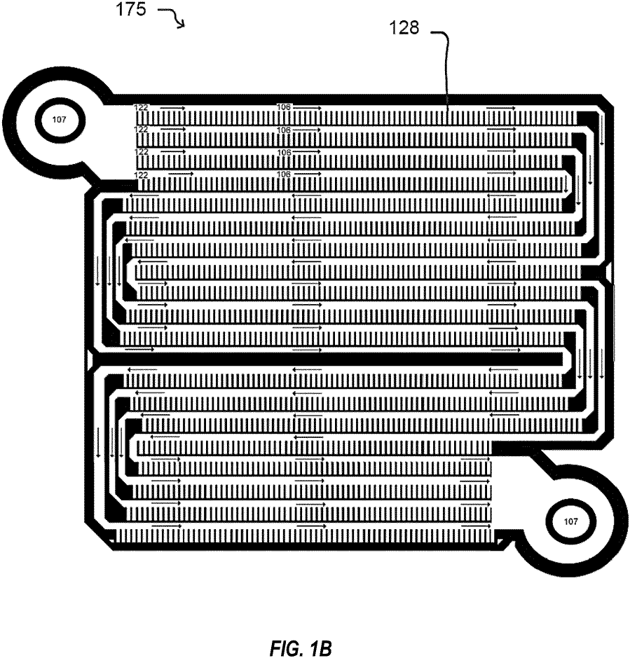

Microfluidic device/system feature cross- applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n-1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

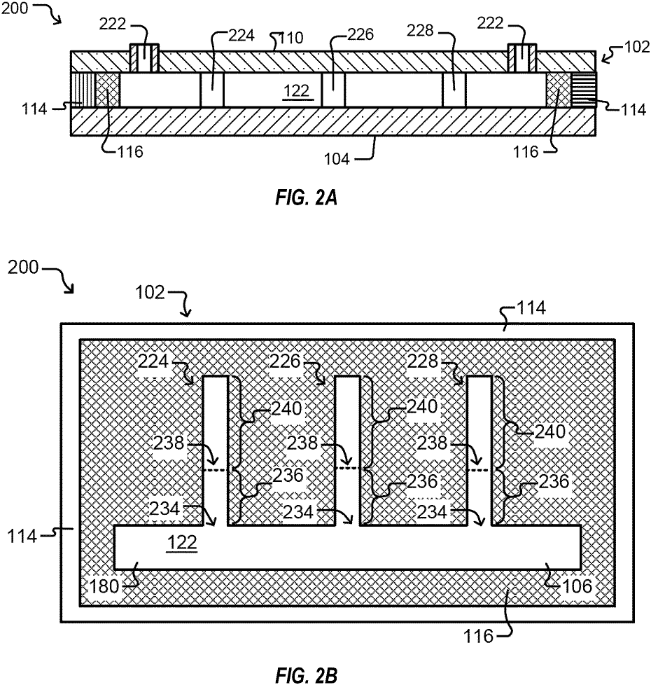

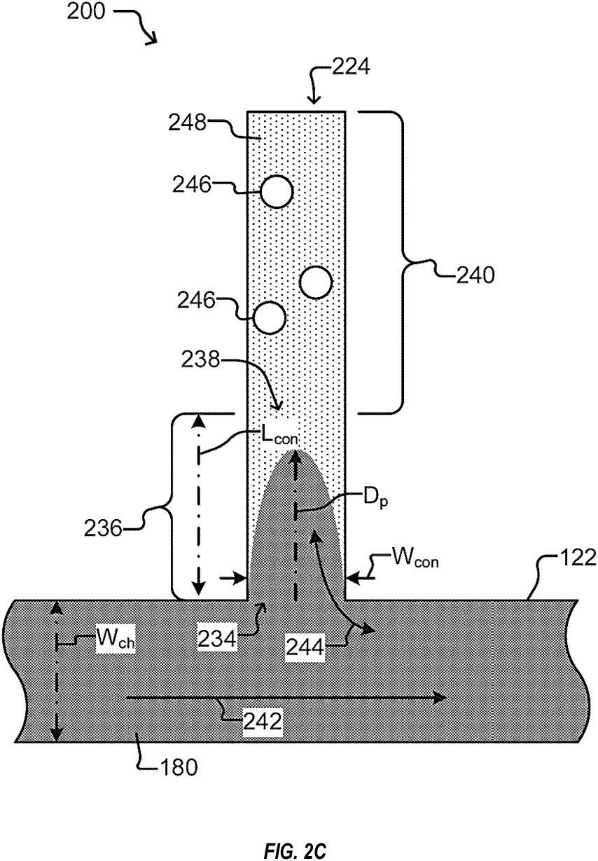

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%. 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-obj ects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

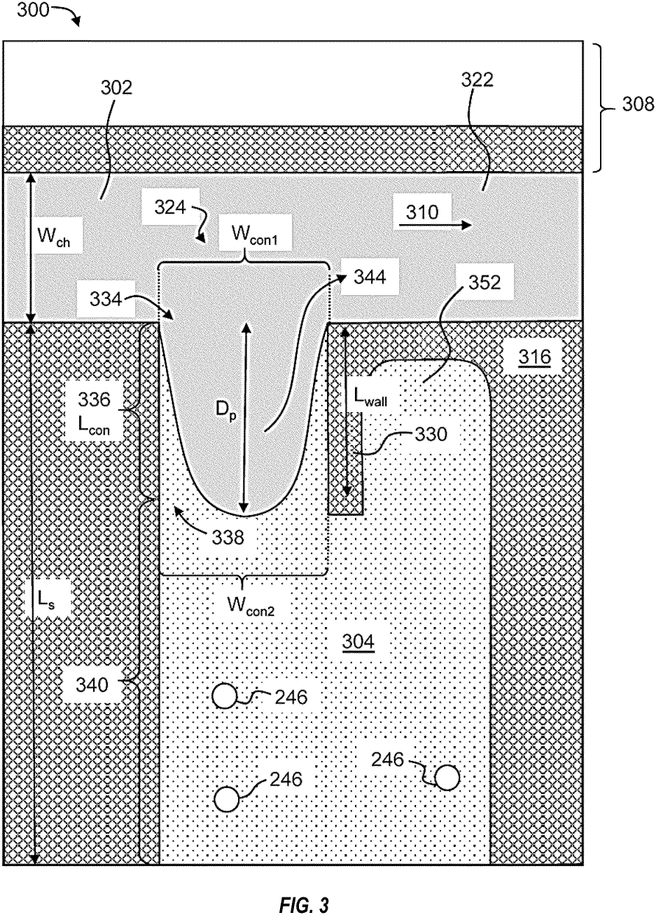

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Lₛ, a connection region 336, and an isolation region 340, where the isolation region 340 contains a second fluidic medium 304. The connection region 336 has a proximal opening 334, having a width Wₑₐₙ₁, which opens to the microfluidic channel 322, and a distal opening 338, having a width Wcon₂, which opens to the isolation region 340. The width Wcon₁ may or may not be the same as W_{con2}, as described herein. The walls of each sequestrationpen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestrationpen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestrationpen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n-1 openings can be valved. When the n-1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/ selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or Wcon1) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or Wcon1) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or Wcon1) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or Wcon₁) of the proximal opening. Thus, for example, the proximal opening of the connection region of a sequestration pen may have a width (e.g., W_{con} or Wcon₁) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or Wcon₁) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or Wcon₁) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or Wcon₁) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or Wcon₁) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or wcon₁) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (Wcon or Wcon₁) of the proximal opening of the connection region of a sequestration pen is less than the width (W_{ch}) of the microfluidic channel. In some embodiments, the width (Wcon or Wcon₁) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (Wcon or Wcon1) of the proximal opening of the connection region of the sequestration pen.

In some embodiments, the size We (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size Wo (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently reentering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion D₀ of the molecule. For example, the D₀ for an IgG antibody in aqueous solution at about 20° C. is about 4.4×10⁻⁷ cm²/sec, while the kinematic viscosity of cell culture medium is about 9×10⁻⁴ m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or Wcon₁) that is 2.0 times or less (e.g., 2.0, 1.9, 1.8, 1.5, 1.3, 1.0, 0.8, 0.5, or 0.1 times) the height (e.g., H_{ch}) of the flow region/ microfluidic channel at the proximal opening, or has a value that lies within a range defined by any two of the foregoing values.

In some embodiments, the width Wcon₁ of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width Wcon₂ of the distal opening, and Wcon₁ and/or Wcon₂ may be selected from any of the values described for W_{con} or Wcon₁. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20 -250 microns, about 20-200 microns, about 20-150 microns, about 20-100 microns, about 30-250 microns, about 30-200 microns, about 30- 150 microns, about 30-100 microns, about 30-80 microns, about 30-50 microns, about 45-250 microns, about 45-200 microns, about 45-100 microns, about 45- 80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or Wcon₁) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10^{8,} 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/ expansion/ movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes ( including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w} <100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules: second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

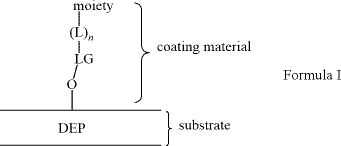

or

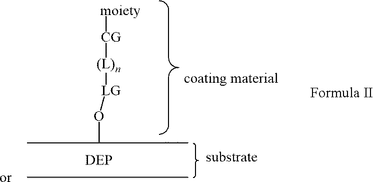

The coating material may be linked covalently to oxides of the surface of a DEP-configured or EW- configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Pat. Application Publication No. US2016/0312165 (Lowe, Jr., et al.), U.S. Pat. Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. Application No. 9,533,306 (Chiou, et al.), each of which is incorporated herein by reference in its entirety. Examples of suitable optically-actuated electrokinetic devices that include combined OET/OEW configured devices can include those illustrated in U.S. Pat. Application Publication No. 2015/0306598 (Khandros, et al.), U.S. Pat. Application Publication No 2015/0306599 (Khandros, et al.), and U.S. Pat. Application Publication No. 2017/0173580 (Lowe, et al.), each of which is incorporated herein by reference in its entirety.

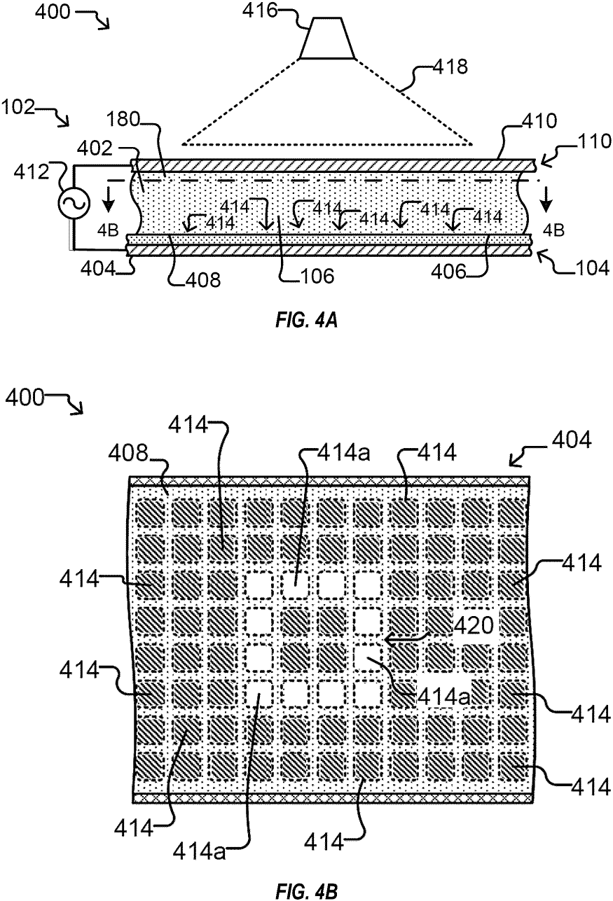

It should be understood that, for purposes of simplicity, the various examples of FIG. 1-5B may illustrate portions of microfluidic devices while not depicting other portions. Further, FIG. 1-5B may be part of, and implemented as, one or more microfluidic systems. In one nonlimiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 200 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100 * the number of hydrogen atoms / the total number of hydrogen and silicon atoms). The layer of a-Si:H can have a thickness of about 500 nm to about 2.0 µm. In such embodiments, the DEP electrode regions 414 can be created anywhere and in any pattern on the inner surface 408 of the electrode activation substrate 406, in accordance with the light pattern 418. The number and pattern of the DEP electrode regions 214 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-obj ect (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Pat. Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2 ° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

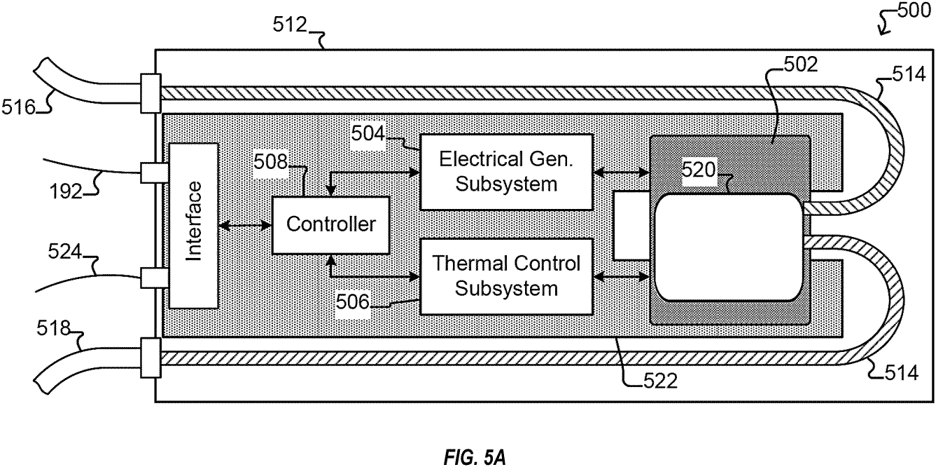

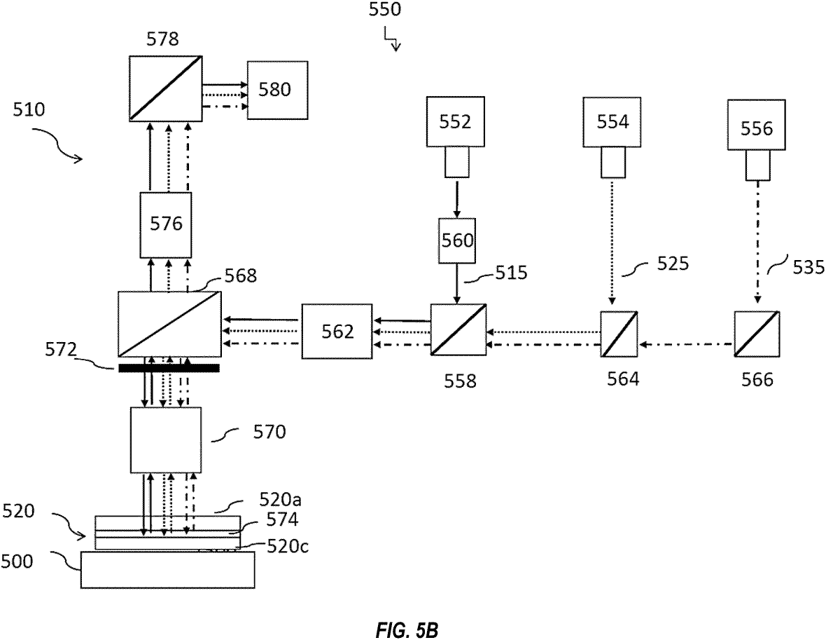

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electrowetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5 V to -6.5 V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 5A the controller 508 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns × 5 microns to about 10 microns ×10 microns, or any values therebetween. The structured light modulator 560 can include an array of mirrors (or pixels) that is 2000 × 1000, 2580 × 1600, 3000 × 2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 5338 and be transmitted therefrom to the first beam splitter 5338, and onward to the first tube lens 5381. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U.S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattem, into each of the plurality of sequestration pens in the sample plane 574 within the field of view. For example, each of the plurality of illumination spots can have a size of about 5 microns × 5 microns; 10 microns × 10 microns; 10 microns × 30 microns, 30 microns × 60 microns, 40 microns × 40 microns, 40 microns × 60 microns, 60 microns × 120 microns, 80 microns × 100 microns, 100 microns × 140 microns and any values there between. The illumination spots may individually have a shape that is circular, square, or rectangular. Altematively, the illumination spots may be grouped within a plurality of illumination spots (e.g., an illumination pattem) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U.S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. Nos. 8,921,055 (Chapman), 10,010,882 (White et al.), and 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### A. Disposing Biological Cells/capture Object Within Chamber

In some embodiments, the method may further include disposing one or more biological cells within the one or more sequestration pens of the microfluidic device. In some embodiments, each one of the one or more biological cells may be disposed in a different one of the one or more sequestration pens. The one or more biological cells may be disposed within the isolation regions of the one or more sequestration pens of the microfluidic device. In some embodiments of the method, at least one of the one or more biological cells may be disposed within a sequestration pen having one of the one or more capture objects disposed therein. In some embodiments, the one or more biological cells may be a plurality of biological cells from a clonal population. In various embodiments of the method, disposing the one or more biological cells may be performed before disposing the one or more capture objects.

In various embodiments, the capture object may be any capture object as described herein. In some embodiments, the capture object may include a magnetic component (e.g., a magnetic bead). Alternatively, the capture object can be non-magnetic.

In some embodiments, a single biological cell is disposed in a sequestration pen. In some embodiments, a plurality of biological cells, for example, 2 or more, 2 to 10, 3 to 8, 4 to 6, or the like, are disposed within said sequestration pen.

In various embodiments, disposing the biological cell may further include marking the biological cell (e.g., with a marker for nucleic acids, such as Dapi or Hoechst stain).

In some embodiments, disposing said biological cell within said sequestration pen is performed before disposing said capture object within said sequestration pen. In some embodiments, disposing said capture object within said sequestration pen is performed before disposing said biological cell within said sequestration pen.

In some embodiments, said enclosure of said microfluidic device comprises at least one coated surface. In some embodiments, the coated surface comprises a covalently linked surface. In some embodiments, the coated surface comprises a hydrophilic or a negatively charged coated surface. The coated surface can be coated with Tris and/or a polymer, such as a PEG-PPG block copolymer. In yet other embodiments, the enclosure of the microfluidic device may include at least one conditioned surface.

The at least one conditioned surface may include a covalently bound hydrophilic moiety or a negatively charged moiety. A covalently bound hydrophilic moiety or negatively charged moiety can be a hydrophilic or negatively charged polymer.

In some embodiments, said enclosure of the microfluidic device further comprises a dielectrophoretic (DEP) configuration, and wherein disposing said biological cell and/or disposing said capture object is performed by applying a dielectrophoretic (DEP) force on or proximal to said biological cell and/or said capture object.

In some embodiments, said microfluidic device further comprises a plurality of sequestration pens. Optionally, the method further comprises disposing a plurality of said biological cells within said plurality of sequestration pens.

A plurality of said biological cells disposed within said plurality of sequestration pens may have substantially only one biological cell disposed within sequestration pens of said plurality. Thus, each sequestration pen of the plurality having a biological cell disposed therein will generally contain a single biological cell. For example, less than 10%, 7%, 5%, 3% or 1% of sequestration pens occupied by a cell may contain more than one biological cell. In some embodiments, the plurality of biological cells may be a clonal population of biological cells.

A plurality of said capture objects disposed within said plurality of sequestration pens may have substantially only one capture object disposed within sequestration pens of said plurality. Thus, each sequestration pen of the plurality having a capture object disposed therein will generally contain a single capture object. For example, less than 10%, 7%, 5%, 3% or 1% of sequestration pens occupied by a capture object may contain more than one capture object.

A plurality of said biological cells and a plurality of capture objects disposed within said plurality of sequestration pens may have substantially only one biological cell and substantially only one capture object disposed within sequestration pens of said plurality. Thus, each sequestration pen of the plurality having a biological cell and a capture object disposed therein will generally contain a single biological cell and a single capture object. For example, less than 10%, 7%, 5%, 3% or 1% of sequestration pens occupied by a cell and a capture object may contain more than one biological cell or more than one capture object. In some embodiments, the plurality of biological cells may be a clonal population of biological cells.

### XI. Biological Cell

In various embodiments, the biological cell may be a single biological cell. Alternatively, the biological cell can be a plurality of biological cells, such as a clonal population. Biological cells include eukaryotic cells, plant cells, animal cells, such as mammalian cells, reptilian cells, avian cells, fish cells, or the like, or prokaryotic cells, bacterial cells, fungal cells, protozoan cells, or the like.

In some embodiments involving first and second biological cells, the first and second biological cells are of the same cell type (e.g., differentiation status). In some embodiments, the first and second biological cells are of the same biological species. In some embodiments, the first and second biological cells are isolated from the same subject, sample, or cell line. In some embodiments, the first and second biological cells are members of the same clonal population.

In some embodiments, the biological cell is from a cell line.

In some embodiments, the biological cell is a primary cell isolated from a tissue, such as blood, muscle, cartilage, fat, skin, liver, lung, neural tissue, and the like.

In some embodiments, the biological cell may be an immune cell, for example a T cell, B cell, NK cell, macrophage, dendritic cell, and the like.

In some embodiments, the biological cell may be a cancer cell, such as a melanoma cancer cell, breast cancer cell, neurological cancer cell, etc.

In other embodiments, the biological cell may be a stem cell (e.g., embryonic stem cell, induced pluripotent (iPS) stem cell, etc.) or a progenitor cell.

In yet other embodiments, the biological cell is an embryo (e.g., a zygote, a 2 to 200 cell embryo, a blastula, etc.), an oocyte, ovum, sperm cell, hybridoma, cultured cell, infected cell, transfected and/or transformed cell, or reporter cell.

### XII. Kits

A kit is also provided for use in methods of assaying a biological cell such as any of those disclosed herein. In some embodiments, the kit includes a plurality of capture objects described herein. In some embodiments, the kit includes: a microfluidic device comprising an enclosure, where the enclosure includes a flow region and a plurality of sequestration pens opening off of the flow region; and capture objects described herein.

In some embodiments, the kit includes: (i) a microfluidic device having a plurality of chambers, and (ii) a plurality of capture objects, each having a plurality of first and second oligonucleotides described herein. In some embodiments, the plurality of capture objects includes capture objects having at least 10 different barcodes (e.g., at least 12, 14, 16, 18, 20, 25, 30, 40, 50, 60, 70, 80, 90, 100, 150, 200, 250, 500, 1000, or more different barcodes).

Further materials that may be included in the kits include reverse transcription enzyme, USER enzyme, a lytic agent (e.g., a lysis buffer), one or more surface conditioning agents (e.g., for conditioning the inner surfaces of the chip), or any combination thereof.

In some embodiments, the plurality of capture objects are in a solution comprising an RNAse inhibitor. In some embodiments, the RNAse inhibitor is a chemical base RNAse inhibitor. In some embodiments, the plurality of capture objects are stored at a temperature of about 4° C.

### XIII. EXAMPLES

### Example 1: Optimization of Receptor Blocking Assays

### Materials and Methods

System and Microfluidic device: The Beacon system and microfluidic device used in the Example were manufactured by Berkeley Lights, Inc. The system included at least a flow controller, temperature controller, fluidic medium conditioning and pump component, light source for light activated DEP configurations, mounting stage for the microfluidic device, and a camera. The microfluidic device was an OptoSelect™ chip configured with OptoElectroPositioning (OEP™) technology. The microfluidic device included a microfluidic channel and a plurality of NanoPen™ chambers fluidically connected thereto.

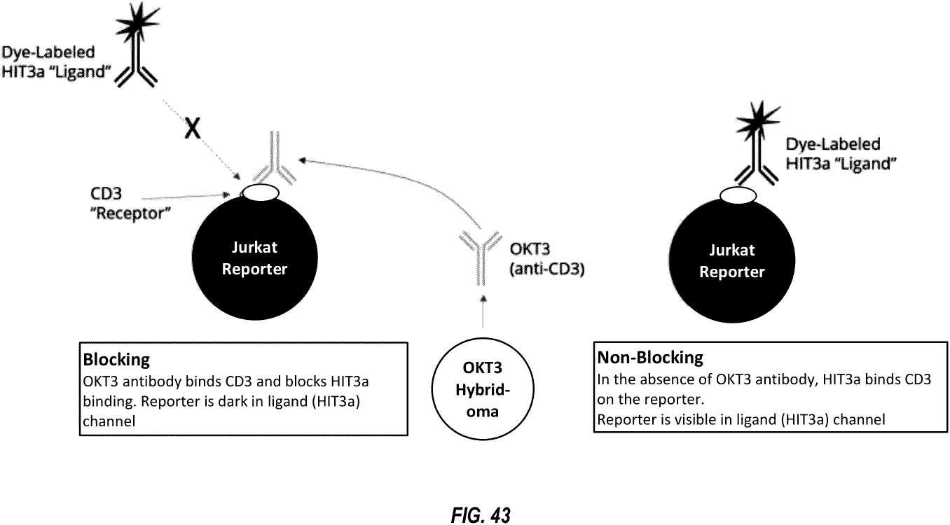

Cell preparation and assay reagents: As shown in FIG. 43, CD3 present on Jurkat cells was used as a model antigen (i.e., the first molecule in this experiment), Jurkat cells (ATCC, TIB-152) were selected as an endogenously expressing reporter cell (i.e., the micro-object, e.g., reporter cells, having the first molecule,in this experiment). OKT3 hybridoma cells (ATCC, CRL-8001) were selected as the anti-CD3 secreting cell and OKT8 hybridoma cells (ATCC, CRL-8014) were used as a negative control cell. A different anti-CD3 antibody clone (HIT3a) (Alexa Fluor 647) was selected as the model ligand (i.e., the second molecule, in this experiment).

Ligand Titration and Incubation Timing. In this experiment, the dye-labeled ligand (AF647 HIT3a) was titrated from low to high concentration and incubated with previously penned Jurkat cells to determine the optimal ligand concentration giving the highest reporter signal with minimal unbound fraction. Briefly, Jurkat cells were imported and penned in selected fields of view (FOVs). Anti-CD3 antibodies (HIT3a) were imported at a concentration of 3.1 nM (0.5 ug/mL), 6.3 nM (0.9 ug/mL), 12.5 nM (1.9 ug/mL), 25 nM (3.8 ug/mL), 50 nM (7.5 ug/mL), and 100 nM (15 ug/mL) respectively in different experiments of this example. Then, time lapse images were taken in the CY5 channel for analyzing the mean pixel intensity of the background area surrounding the detected cells (MeanBackgroundBrightness) and the maximum pixel intensity of the detected cells (MaxBrightness) by using TPS (Target and Pen Selection) analysis of the Beacon system. Additional details of TPS and associated detection methods are described in International Application Publication No. WO2016/094459, filed on Dec. 8, 2015; WO2018/102748, filed on Dec. 1, 2017; and WO2019/232473, filed on Mary 31, 2019, each of which disclosures are herein incorporated by reference in its entirety for any purpose. An additional metric “Max - BG” was calculated as the difference between MaxBrightness and MeanBackgroundBrightness to determine the background subtracted brightness of the reporter cell.

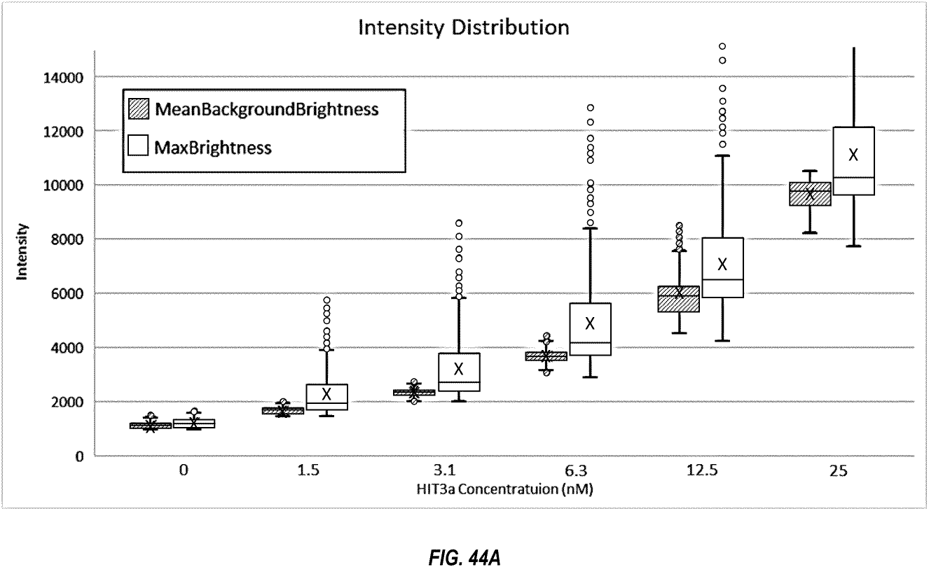

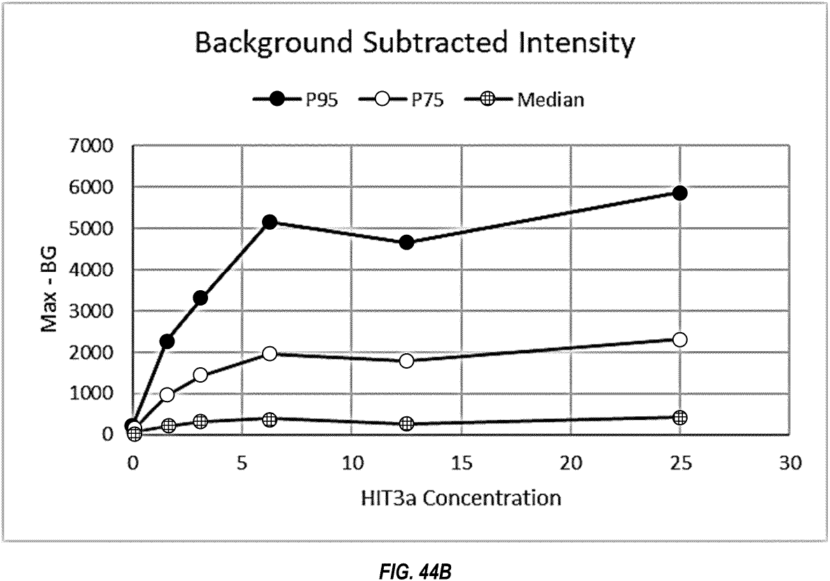

The results are shown in FIGS. 44A-44B. As the concentration of the ligand increases, both the background and reporter cell signals increase (FIG. 44A). However, the background subtracted signal (Max - BG) plateaus when HIT3A concentration was increased above 6 nM (FIG. 44B) . Due to the Jurkat cell heterogeneity observed above, the median, 75th, and 95th percentile of the background subtracted signal were included in this analysis, and all show the same plateau at around 6 nM. This suggests that above 6 nM, the fraction of unbound ligands is increasing, which serves only to decrease signal to noise.

Reporter Expression and Heterogeneity. In this experiment, reporter expression and heterogeneity were explored more thoroughly at the optimized ligand concentration determined as above. Additionally, to increase reporter cell loading density and improve uniformity, the Jurkat cell import density was increased from 5.6×10^6 to 1.1×10^7 cells/ml, and the standard import was replaced with the “Well Import”. The reporter cells were penned en masse, since all FOVs were to receive the same treatment with ligand. After the reporter cells were imported, the chip was placed in a vertical orientation for 5 min such that the cells would passively settle into the pens, and then the chip re-established in a horizontal orientation for the remainder of the experiment. At the 1.7×10^7 cells/mL import density used, 20% (2218) of the pens did not receive a reporter cell, and 45% (5045) of the pens received 2 or more reporter cells.

After loading the reporter cells, the AF647 HIT3a ligand (1 ug/ml, 6.7 nM) was imported and incubated with time lapse imaging as previously described. After 30 minutes of incubation, the chip was flushed with 500 uL of culture media followed immediately by Pulse Culture, described in earlier sections of this application, for an additional 25 minutes. The flush and Pulse Culture were performed to determine if removing unbound ligand would result in decreased background and improved reporter cell signal. Because the ligand is an 1gG antibody, in this model system, the 25 minutes of Pulse Culture allowed the ligand to diffuse out of the pens.

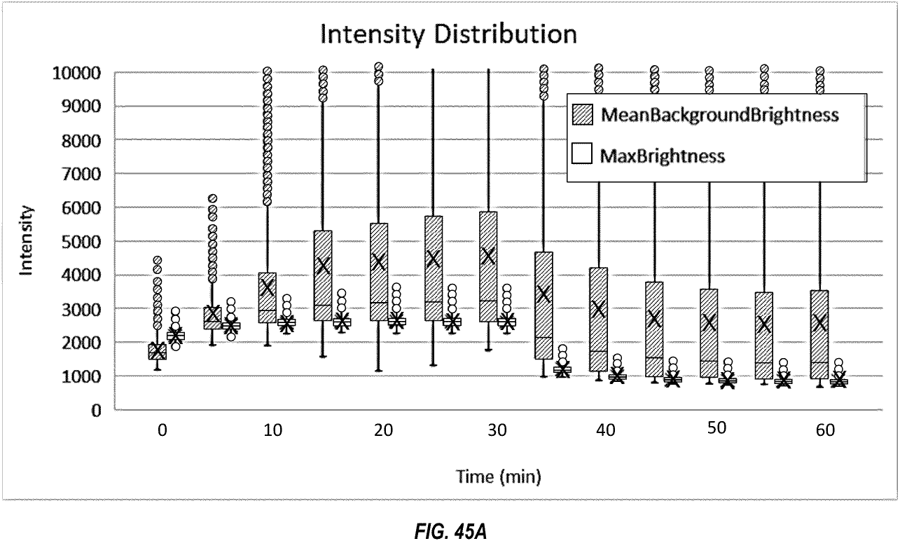

TPS was used to process the image sequences, as described above. FIG. 45A shows a time course of background (MeanBackgroundBrightness) and reporter cell intensity (MaxBrightness). The background subtracted reporter cell signal “BG-Max” was also calculated in FIG. 45B. The background subtracted reporter cell signal increases rapidly in the first 15 minutes, then gradually stabilizes. At 30 minutes the chip was flushed, and the background rapidly dropped off, with only a small temporary increase in background-subtracted cell signal.

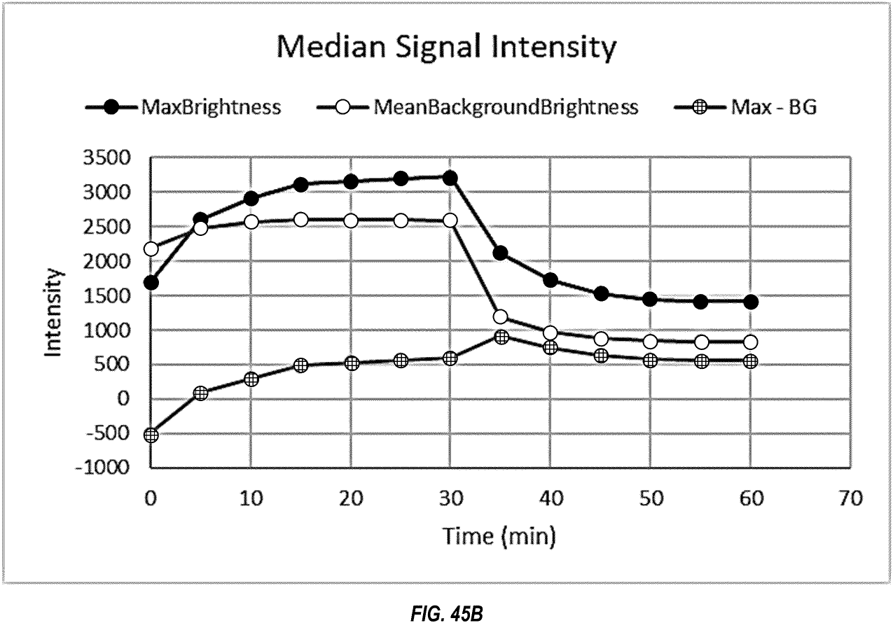

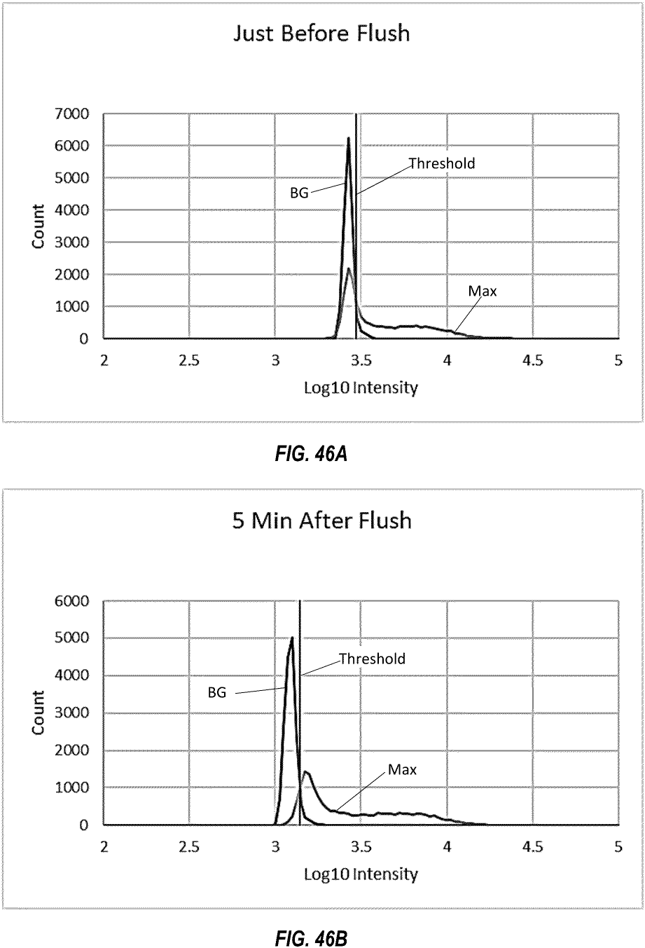

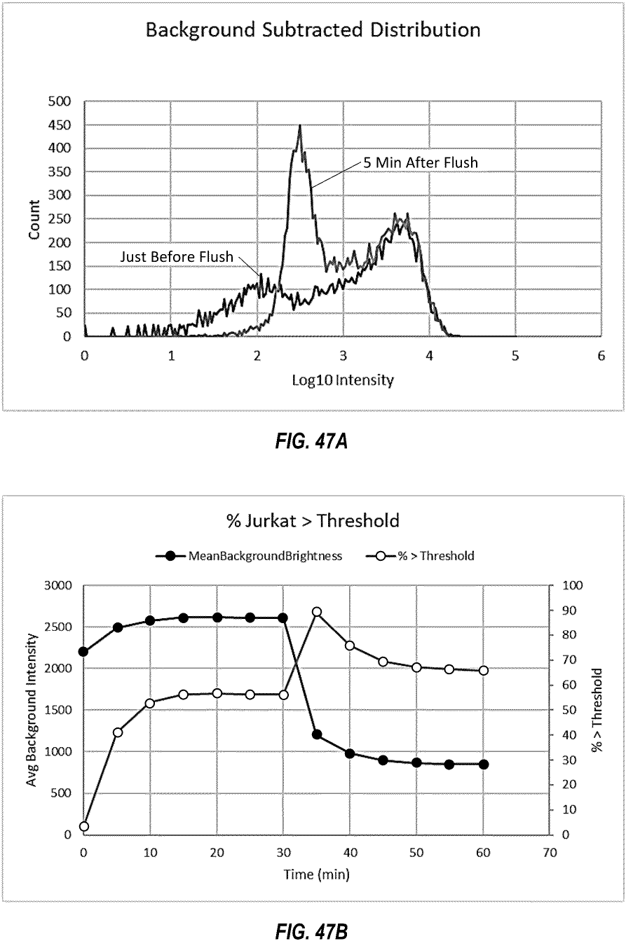

Mean Background Brightness (CY5) and MaxBrightness (CY5) distributions are plotted in FIGS. 46A-46B, just before and just after the flush at the 30 min timepoint. There is significant overlap between the two distributions which indicates a large fraction of the reporter cells is indistinguishable from background. Furthermore, the background subtracted signal distributions from just before and just after the flush are plotted in FIG. 47A. The population looks bimodal, with a sizable fraction likely undetectable above background, in agreement with what was observed visually in FIG. 46 above. A reporter cell detection threshold was set by adding 2 standard deviations to the average background signal at each timepoint. The average background and fraction of detectable cells are plotted as a function of incubation time (FIG. 47B). The fraction of detectable Jurkat cells increased rapidly to 56% during the pre-flush incubation. After flushing, the background dropped, resulting in a transiently high 89% of detectable Jurkat cells. Further flushing resulted in a stabilized detectable fraction of 66%.

As discussed previously, reporter cell populations with a sizable fraction of low or undetectable signal can result in an increased false positive blocking hit rate, since positive blocking is indicated by dark reporter cells. At the stabilized 66% detection rate above, one would expect a 34% false positive blocking hit rate if only 1 reporter cell was added to each pen. Increasing the reporter cells to 2 cells per pen, at a 66% detection rate, reduces the expected false positive rate for each pen from 34% to 12%, since the probability of both cells being below the detection threshold is 0.34² = 0.12, or 12%. Generally, the false positive rate can be described by the formula:

$$F P \text{=} {\left(1 - d\right)}^{n}$$

where FP is the false positive rate, d is the detection rate of a ligand-bound reporter cell, and n is the number of reporter cells per pen.

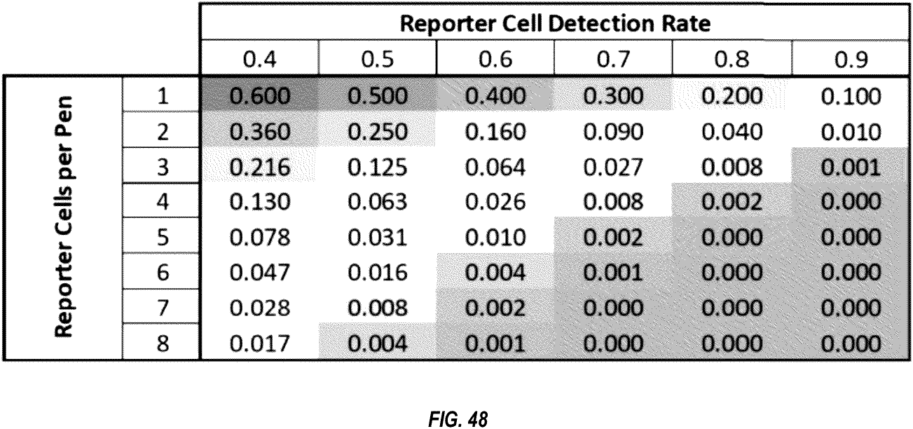

FIG. 48 shows the expected false positive blocking hit rates for a range of reporter cell detection rates and the number of reporter cells per pen. Thus, a reporter cell population with a high detection rate is preferred, while more heterogeneous reporter populations can be used if the in-pen reporter density is increased.

### Example 2: Receptor Blocking Antibody Screening

### System and Microfluidic Device: Same as in Example 1.

Cell preparation and assay reagents: Same as in Example 1, except that the Jurkat cell import density was further increased to 2.5×10^7 cells/ml, to target a greater per-pen load density.

An OptoSelect 11k chip was loaded and primed with hybridoma load media, according to standard procedures. OKT8 (2.3×10^6 cells/ml) and OKT3 (2.0×10^6 cells/ml) hybridoma cells were sequentially imported and penned. For demonstration purposes, the penning parameters were selected such that the two cell types were loaded in alternating pens with an empty pen between all cells. After penning, the chip was primed with hybridoma culture media and cultured overnight at 36° C. to allow for some expansion of single cell loads and increased secretion for the pending assays.

The Well Import and Off Instrument Loading were again used for loading the reporter cells. The chip was imaged in brightfield, and cells counted to assess the reporter cell load distribution on chip. 70% of the pens had 3 or more Jurkat cells per pen, which, assuming the same 66% detection rate measured previously, would result in a predicted false positive blocking rate of 4% or less. 17% of the pens had 2 Jurkat cells, which would have a false positive rate of 12%. 10% of the pens had only 1 Jurkat cell, which would result in a 34% false positive rate.

After loading, the reporter cells were incubated for 30 min with the already penned hybridoma cells, using Pulse Culture at 36° C. to minimize pen-to-pen diffusion of secreted antibodies. Default pulsed-culture settings were used: 36° C. culture temperature and 4 uL flush every 2 min. This incubation period allows secreted antibodies to bind the reporter cells prior to introduction of the dye-labeled ligand.

After the 30 min incubation, the AF647 HIT3a ligand (1 ug/ml, 6.3 nM) was imported and incubated with time lapse imaging as previously described. After an additional 30 min incubation, the chip was flushed with 500 uL of culture media followed immediately by Pulse Culture for 25 min. Upon completion of the blocking assay, an IgG binding assay was performed to confirm hybridoma IgG secretion.

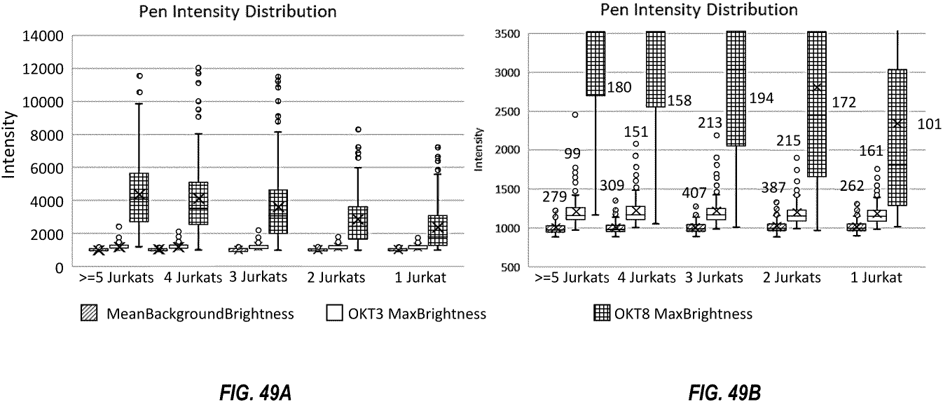

TPS analysis was used to process the image sequences, as described above. Background (MeanBackgroundBrightness) and reporter cell intensity (MaxBrightness) were tabulated, and their distributions plotted as a function of hybridoma type and Jurkat cell load (FIGS. 49A-49B). As shown in FIGS. 49A-49B, the reporter cell signal (MaxBrightness) from secreting OKT3 pens was only slightly higher than background, indicating blocking of HIT3a anti-CD3, as expected. Reporter cell signal from secreting OKT8 pens, on the other hand, was significantly higher than both background and the OKT3 pens, indicating a negative blocking result. A gradual decrease in reporter cell signal was observed in the non-blocking OKT8 pens with decreasing reporter cell load. This is most likely due to the heterogeneity in the reporter cell population, as discussed above. When five Jurkat cells were loaded, the probability of a “low signal” for the pen was less than 1%. However, when only one Jurkat cell was loaded in a pen, the probability of getting a “low signal” for the pen is ~34%, for this reporter cell preparation.

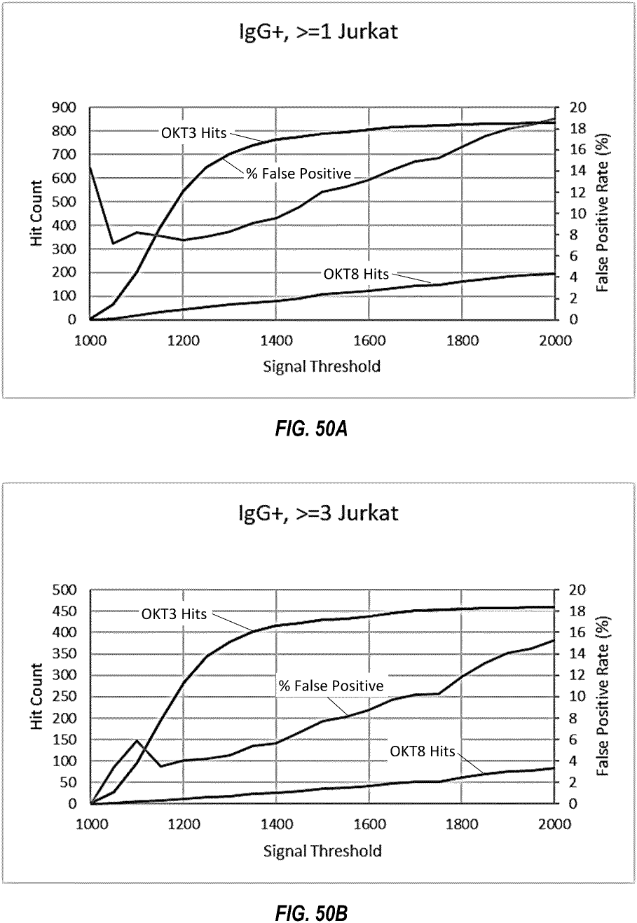

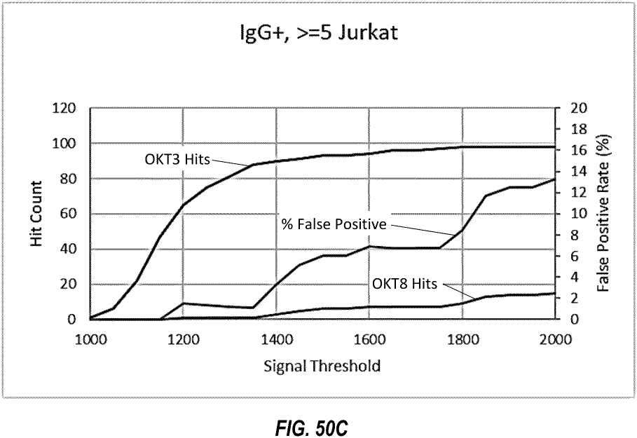

There is no specific method for establishing an exact signal threshold for the highest true positive hit recovery with the lowest possible false positive rate. Lowering the signal threshold and/or limiting the candidate pens to those with a higher number of reporter cells will lower the risk of including false positives. However, this comes with the cost of excluding some true positives. Conversely, raising the signal threshold and/or including pens with fewer reporter cells will increase both the number of true and false positives. This concept is demonstrated for the model blocking assay in FIGS. 50A-50B.

Regardless of reporter cell load, increasing the signal threshold results in an increase in both the number of true positive hits and the false positive hit rate. However, as discussed above, limiting the candidate pens to those with a higher number of reporter cells decreases the false positive rate at the cost of a total number of true positive hits. The following table shows the number of true positives and false positive rate for this data set, at a fixed signal threshold of 1400.

TABLE 5

| Jurkat Cells Per Pen | Total Hits Selected | True Hits | False Positive Rate (%) |
|---|---|---|---|
| >=1 | 843 | 762 | 9.6 |
| >=3 | 440 | 415 | 5.7 |
| >=5 | 93 | 90 | 3.2 |

A general approach to generating a hit list is to sort the pens based on the MaxBrightness of the reporter cells. Pens with lower reporter cell signals are most likely to be true blockers. Pens with higher signals are most likely non-blockers. If the reporter cell characterization has been performed as recommended, the false positive risk as a function of reporter cell load count can be determined beforehand. For an assay with a very low hit rate, it would be reasonable to include pens at higher risk of false positive hits (fewer reporter cells) in order to unload and recover as many real hits as possible. On the other hand, if the assay results in a high hit rate, it would be reasonable to exclude pens with higher risk of being a false positive, since there are plenty of pens from which to choose.

### Example 3. Ligand/Receptor Blocking Antibody Screening

### Materials and Methods

System and Microfluidic device: the system and microfluidic device used in the Example were manufactured by Berkeley Lights, Inc. The system included at least a flow controller, temperature controller, fluidic medium conditioning and pump component, light source for light activated DEP configurations, mounting stage for the microfluidic device, and a camera. The microfluidic device was an OptoSelect™ chip configured with OptoElectroPositioning (OEP™) technology. The microfluidic device included a microfluidic channel and a plurality of NanoPen™ chambers fluidically connected thereto.

Cell preparation and assay reagents: Primary plasma cells were isolated from the bone marrow and spleen of Balb/c mice immunized with Fc-fused PD-L1 extracellular domain (huPD-L1 ECD-FC) using a CD138+ plasma cell isolation kit (Miltenyi Biotech). PD-1-AF488 was prepared by labeling a recombinant PD-1-Fc fusion protein (ChemPartner) using an AF488 labeling kit (Thermo Fisher Scientific). Recombinant PD- L1 beads were prepared by coupling biotinylated PD-L1 (ChemPartner) to streptavidin polystyrene particles (Spherotech Inc.). Finally, CHO-K1 cells were engineered to over-express human PD-L1 (ChemPartner).

Antibody screening assays: Single plasma cells were loaded into individual NanoPen™chambers on OptoSelect™ 11k chips using Berkeley Lights’ OEP™ technology. CHO-K1-PD-L1 cells were then bulk loaded into individual NanoPen chambers so that an average of 4 cells were loaded per pen. An assay mixture of antigen-coated beads and secondary antibody were loaded to simultaneously perform a recombinant PD-L1 bead binding assay (in-channel) and cell binding assay (in- pen). The assay mixture was then flushed out of the chip to perform the ligand/receptor-blocking assay. Cell-based assays were scored by human verification.

Recombinant PD-L1 bead binding assay (in-channel): PD-L1 coated beads, in suspension with a fluorescently labeled anti-mouse secondary antibody (AF568), were imported into the main channel of the OptoSelect 11k chip so that beads were concentrated around the mouth of each NanoPen chamber. Secreted antibodies diffused from the NanoPen chambers into the channel where binding of the secreted antibody was detected optically as in-channel “blooms” in the TRED imaging channel. Blooms observed over the center of the NanoPen indicated positive PD-L1 binding.

Cell binding assay (in-pen): The in-pen cell binding assay was performed by first co-incubating plasma B cells and CHO-K1-PD-L1 cells for 1 hour to allow for secreted antibodies to saturate the receptors. A fluorescently labeled anti-mouse secondary antibody (AF568) was then perfused through the OptoSelect 11k chip and allowed to diffuse into the NanoPen chambers. Anti-PD-L1 cell-binding antibodies were identified by locating pens with fluorescent CHO-K1- PD-L1 cells when imaged on the Beacon system using a TRED filter cube.

Ligand/receptor-blocking assay (in-pen): After completing the in-pen cell binding assay, a fluorescently labeled, soluble PD-1-Fc fusion protein (AF488) was perfused through the OptoSelect 11k chip. PD-1 binding to the reporter cells was detected in the FITC imaging channel. NanoPen chambers containing CHO-K1-PD-L1 cells that are positive in both the TRED and FITC channels confirm the presence of secreted antibodies that have PD-L1 binding, but no blocking activity. NanoPen chambers that contained CHO-K1-PD-L1 cells that were positive in TRED but negative in FITC contained secreted antibodies that had both PD-L1 binding and PD-1/PD-L1 blocking activity.

Sequence recovery and functional confirmation: Cells secreting PD-L1/PD-1 blocking antibodies were exported from specific NanoPen chambers to a 96-well PCR plate. Antibody heavy and light chain sequences were amplified and recovered using components of the Opto™ Plasma B Discovery cDNA Synthesis Kit and the Opto™ Plasma B Discovery Sanger Prep Kit, Mouse (Berkeley Lights). Sample preparation and sequencing was performed as described in International Application Publication No. WO2019191459, entitled “Methods for Preparation of Nucleic Acid Sequencing Libraries, filed on Mar. 28, 2019, the disclosure of which is herein incorporated by reference in its entirety. Recovered sequences were cloned into expression constructs, and antibodies were re- expressed and purified. Antigen binding and blocking activity was confirmed using plate-based ELISA and FACS measurements.

### Results:

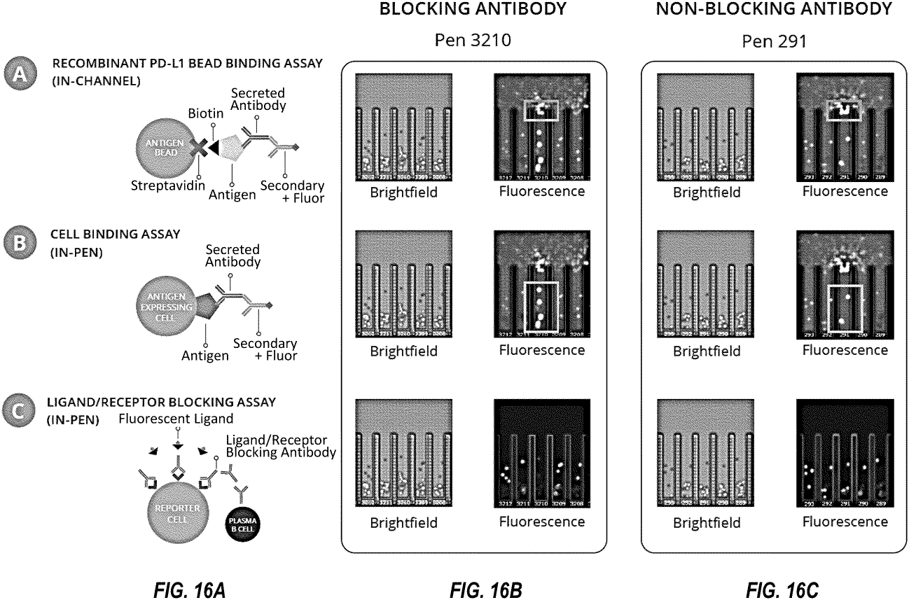

Identifying blocking antibodies using a ligand/receptor blocking assay: An in-channel recombinant protein binding assay (FIGS. 16A-16C, top row) and an in-pen cell binding assay (FIGS. 16A-16C, middle row) were first performed simultaneously to identify antibodies that bound recombinant PD-L1 and native PD-L1 expressed on the cell surface of a reporter cell, respectively. Following the recombinant and cell-based binding assays, a PD-1/PD-L1 ligand/receptor-blocking assay was performed in-pen (FIGS. 16A-16C, bottom row). Fluorescent imaging clearly revealed antibodies that effectively blocked the ability of the fluorescently labeled PD-1 to bind PD-L1 expressed on CHO-K1 cells (FIG. 16B, bottom panel) as well as antibodies that were not effective blockers (FIG. 16C, middle panel) despite binding to PD-L1 in the recombinant and cell-based binding assays (FIGS. 16A-C, top and bottom rows).

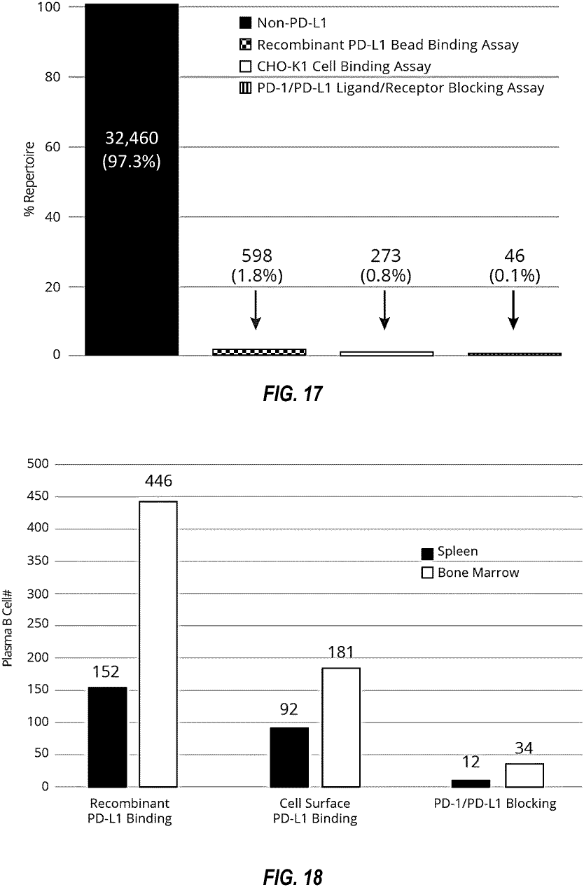

Of the 33,377 mouse plasma B cells screened (16,500 cells from the spleen and 16,877 cells from the bone marrow), 598 (1.8%) cells generated antibodies that bound to the PD-L1 coupled beads. The cell binding assay allowed us to down-select further to 273 (0.8%) cells that secreted antibodies that bound to PD-L1 expressed on the surface of the CHO-K1 cells (FIG. 17). The ligand/receptor- blocking assay identified 46 (0.1%) lead candidates that both bound PD-L1 and were able to block the interaction between fluorescently labeled PD-1 and PD-L1. The ability to down-select to 46 lead candidates eliminated the need to sequence, clone, re-express, and purify nearly 600 antibodies.

Discovering more blocking antibodies by accessing bone marrow plasma B cells: Opto Plasma B Discovery is uniquely capable of accessing plasma B cells from multiple organs, including spleen, bone marrow, and lymph nodes. 3x more blocking antibodies were identified by screening plasma B cells from bone marrow compared to the spleen plasma B cells (FIG. 18), suggesting that this B cell compartment could be an important source for therapeutic molecules. The plasma B cells secreting PD-1/PD-L1 blocking antibodies were unloaded from the chip for cDNA recovery and amplification of antibody heavy/light chain genes for sequencing. Sequencing the PD-1/PD-L1 blocking antibodies confirmed that the lead candidates that were identified using the Beacon instrument were unique antibodies compared to commercially-approved antibodies currently in the clinic (not shown).

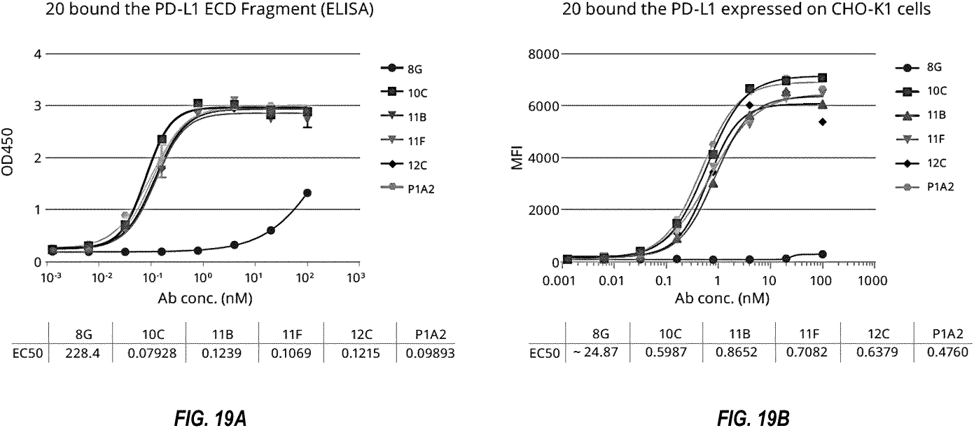

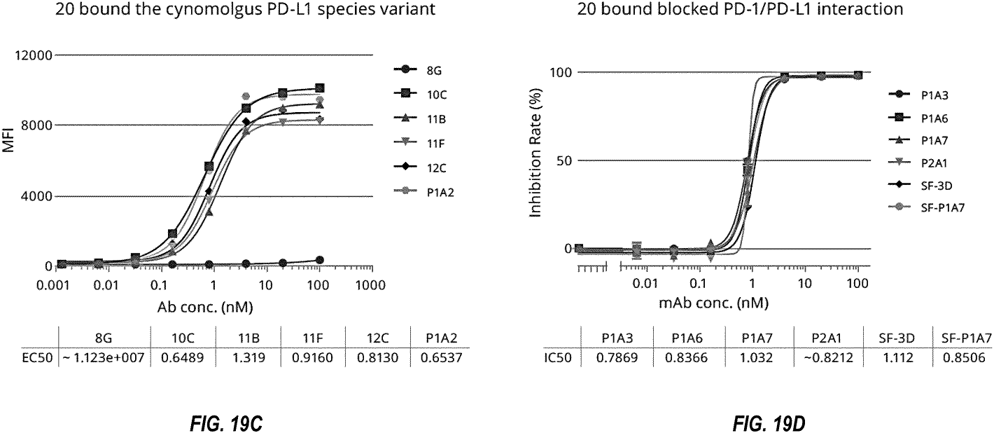

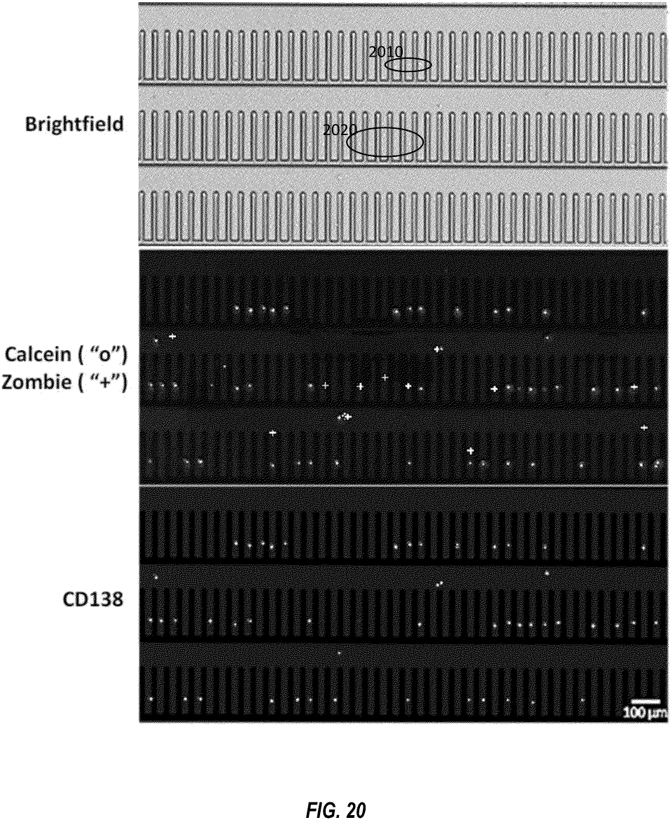

Identifying antibodies with performance comparable to commercially-approved antibodies: Twenty-four (24) blocking antibodies were selected to clone, re-express and purify for characterization using orthogonal assays (FIGS. 19A-19D). Twenty (20) out of 24 antibodies (83%) of antibodies bound the extracellular domain (ECD) of human PD-L1 as confirmed by ELISA (FIG. 19A). This binding was not limited to just recombinant proteins, as 20 of 24 antibodies also bound to CHO-K1 cells expressing the PD-L1 protein (FIG. 19B). It was determined that these candidates bound the cynomolgus PD-L1 variant (FIG. 19C), an important requirement for pre-clinical animal toxicological studies. Finally, it was confirmed the lead candidates had functional ligand/receptor blocking activity in wellplate-based assays (FIG. 19D). Of the 20 antibodies tested, 5 had IC50 values comparable to commercially-available therapeutic antibodies and 2 had sub-nanomolar affinities based on results generated using a Biacore instrument (GE Healthcare, data not shown).

### Example 4: Enhanced Penning of Live Plasma Cells Using Machine Learning Algorithm

A. Cell stain selection to distinguish live and dead cells: Primary plasma cells were isolated from dissected spleens derived from immunized Balb/C mice. Enrichment of the plasma cells from the splenocytes was performed by density gradient centrifugation followed by magnetic-activated cell sorting (MACS) using a commercially available Mouse CD138+ Plasma Cell Isolation Kit (Miltenyi, 130-092-530). The plasma cells were stained with calcein-AM (Bio-Legend, 425201), an esterase activity indicator, as per the manufacturer’s instructions. The cells were also labeled with a PE conjugated anti-mouse CD138 antibody (Miltenyi, 130-120-810) and Zombie Violet Fixable Viability Dye (Biolegend, 423113) at optimized concentrations. The cell staining procedures for each of the stains used were performed as follows:
calcein Stain (Live staining). 1) Resuspend in 50 microliter PBS. 2) Add 0.5 microliter of calcein-AM (calcein: BioLegend 76084, Lot B255562, From Jul. 19, 2020). 3) Incubate at room temperature incubation protected from light, 20 minutes. 4) Pellet cells and resuspend in warm culture media, allow to incubate 10 minutes at RT to ensure optimal retention of calcein-AM. 5) After incubation, calcein-AM labeled cells are ready for downstream applications or analysis.
Zombie Violet Stain for Cells (Dead staining). 1) 1:100 in 50 microliter PBS. 2) In PBS, room temperature incubation for 10 min
CD138 Stain. 1) Dilute AF647 anti-mouse CD138 (Biolegend, 142526) 1:20 in FACS Buffer. 2) Resuspend cells in 200 uL of diluted CD138 stain. 3) Incubate at 4C protected from light, 30 minutes. 4) After incubation, Zombie Violet labeled cells are ready for downstream applications or analysis

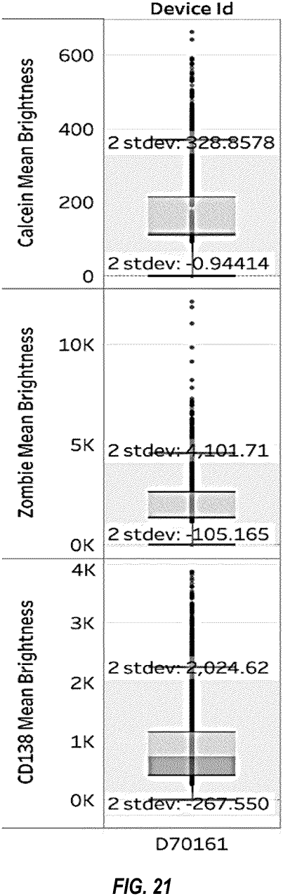

After staining, cells were imported into an OptoSelect™ device (Berkeley Lights, Inc.), configured with OptoElectroPositioning (OEP™)technology operated on a Beacon system (Berkeley Lights, Inc.) and imaged at FITC (calcein), DAPI (Zombie), CY5 (CD138) cube channels. FIG. 20 shows cells stained with calcein, Zombie, CD138. To test whether Beacon system can be able to distinguish live and dead plasma cells as accurately as possible, mean fluorescence levels of cells imported in chip channels were compared between unstained and stained plasma cells. Looking at the negative control (plasma cells without stains), background signals were found in the stains. Out of all three stains, calcein had the most minimal mean background signals (< 1000 AFU). The thresholds for each channel to determine whether cells are stained positive were based on the 2 standard deviations (stdev) above the average for each channel. n = 5837 cells (FIG. 21).

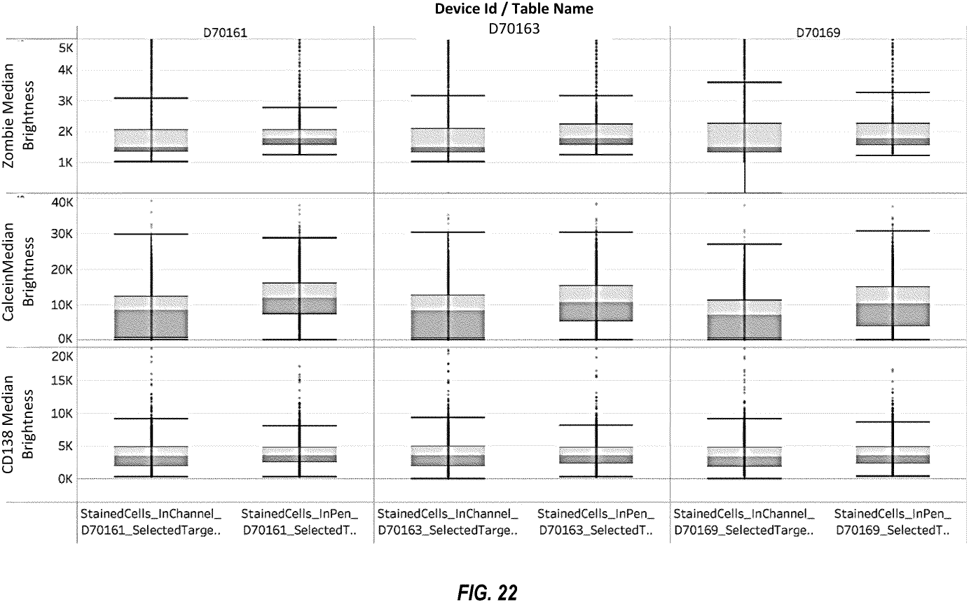

Fluorescence was then examined from the stained plasma cells in channel and in pen after the cell load with OEP. With the same imaging exposure times and setting, boxplots of each stain were examined. Any outliers were eliminated by gating cell diameter (10 micron) and any cell debris/clump verified in Image Analyzer 2.1. Each dot represented a plasma cell in channels. Whiskers extended to data within 1.5 times the IQR. Since dielectrophoretic force from OEP is expected to be higher in live cells than dead cells, cells in the pens would fluoresce high in calcein/CD138 and low in Zombie. Across 3 chips (D70161, n = 4403 in channel, n = 3179 in cells; D70163, n = 4698 cells in channel, n = 3561 cells in pen; D70169, n = 4523 cells in channel, 3563 cells in pen), it was observed that the in-pen cells appear to have higher calcein and Zombie expression levels than the in-channel cells, while CD138 expression level is similar between in-pen and in-channel. FIG. 22 suggests that calcein would be the optimal stain in Beacon system to distinguish live and dead cells.

### Subpopulation Frequency Comparison Between In-pen vs In-channel Cells

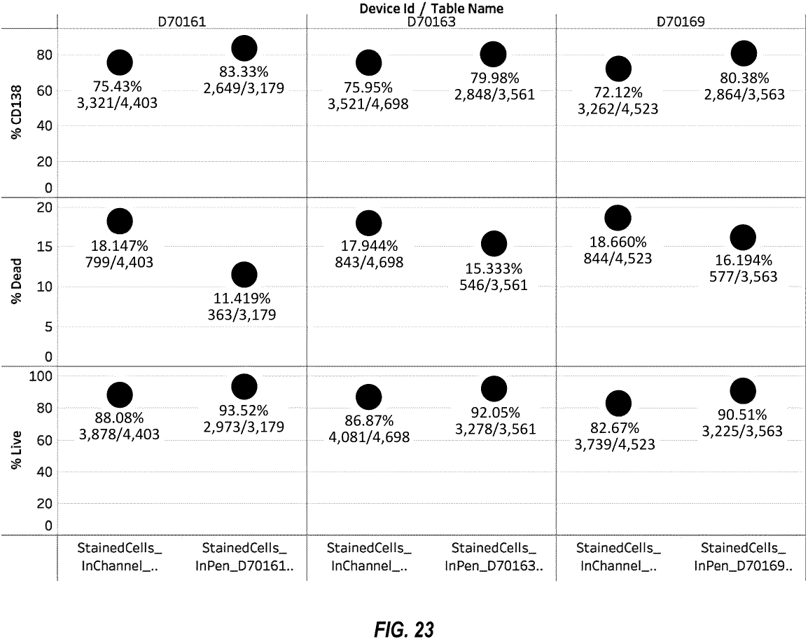

Next, subpopulation frequencies difference between in-channel and in-pen cells were examined. Based on the threshold from unstained cells, as shown in FIG. 23, the CD138+ in-channel subpopulation is lower than the CD138+ in-pen subpopulation. Similarly, the calcein+ (Live) in-channel subpopulation is lower than the calcein+ in-pen subpopulation. Also, Zombie+ (Dead) in-pen subpopulation was seen to be lower than the Zombie+ in-channel subpopulation. The boxplots suggest that enrichment process of live cells by penning cells in pens can be observed with the cell stains with Beacon system.

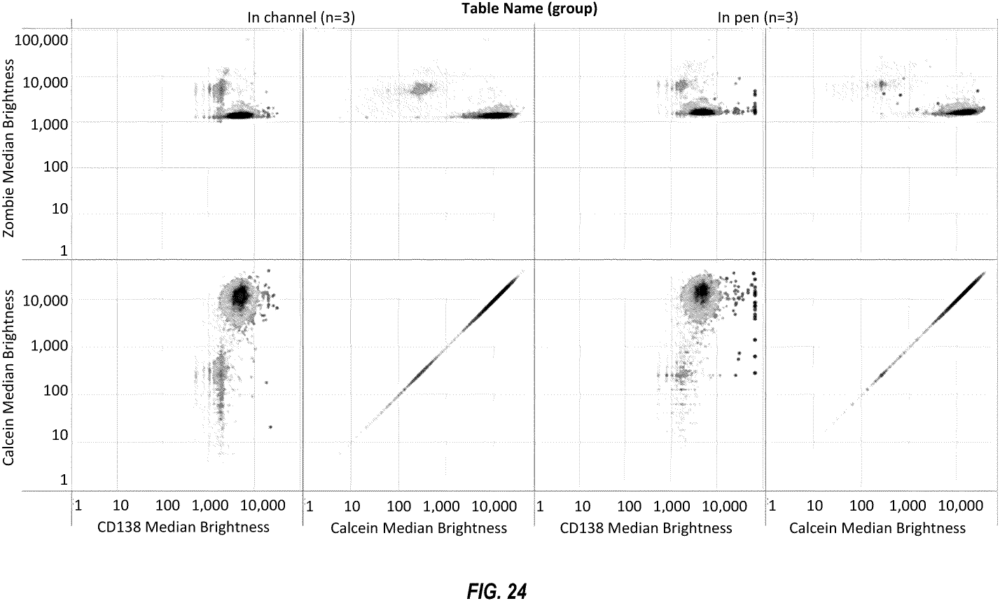

Association among CD138, calcein, Zombie stains: Next, relationships among CD138, calcein, Zombie expression levels were examined. In log scale, it was seen that in the density scatter plot (FIG. 24) that Zombie (dead) and calcein (live) expression levels were separable into 2 subpopulations (one subpopulation with high Zombie and low calcein, the other with low Zombie and high calcein). In addition, comparing between Zombie and CD138, a major subpopulation was observed with high CD138 and low Zombie expression levels. Comparing between calcein and CD138, a major subpopulation with high calcein and high CD138 expression levels was observed. Both in-pen and in-channel samples have similar trends. Within the Beacon system, calcein was seen to separate the live and dead subpopulations with the largest fluorescence separation. The on-chip data match very well with the off-chip flow cytometry data (see the following paragraphs.

Off-Chip FACS analysis of CD138, calcein, Zombie stains: The stained cells were analyzed on a BD FACS Celesta Cell Analyzer, and the data was further analyzed using the FlowJo v10 software. The data from the FACS analysis showed that cells with strong calcein signal had very low to no signal for Zombie Violet, which only stains dead or dying cells. Cells that are expressing CD138, a known plasma cell surface marker, also had strong calcein signal.

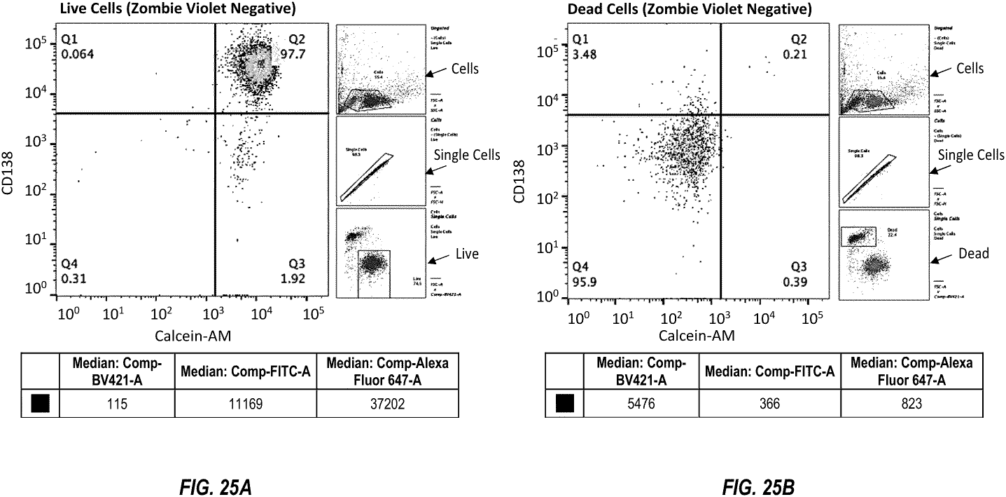

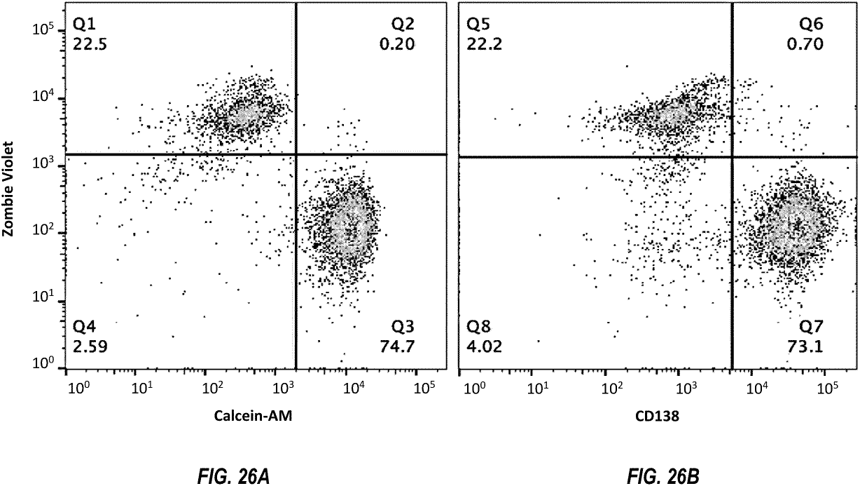

The scatter plots show the signal intensities of live cells or dead cells for CD138 (AF647) and calcein (FITC) (FIGS. 25A-25B). The 3 plots on the right of each panel shows the backgating analysis to show where the target population (enclosed by the solid lines) is located in the parent populations. The table at the bottom shows the Median Fluorescent Intensities (MFI) of Zombie Violet (Comp-BV421-A), calcein (Comp-FITC-A), and CD138 (Comp-AF647-A). FIGS. 26A-26B show the correlation between Zombie Violet (DAPI) vs calcein-AM (FITC) (FIG. 26A) and CD138 (AF647) (FIG. 26B).

### Association Between Brightfield and Fluorescent (calcein Stain) Image Sequences.

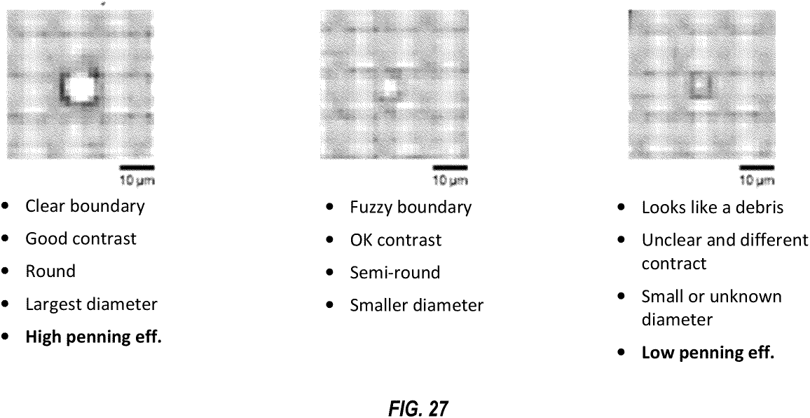

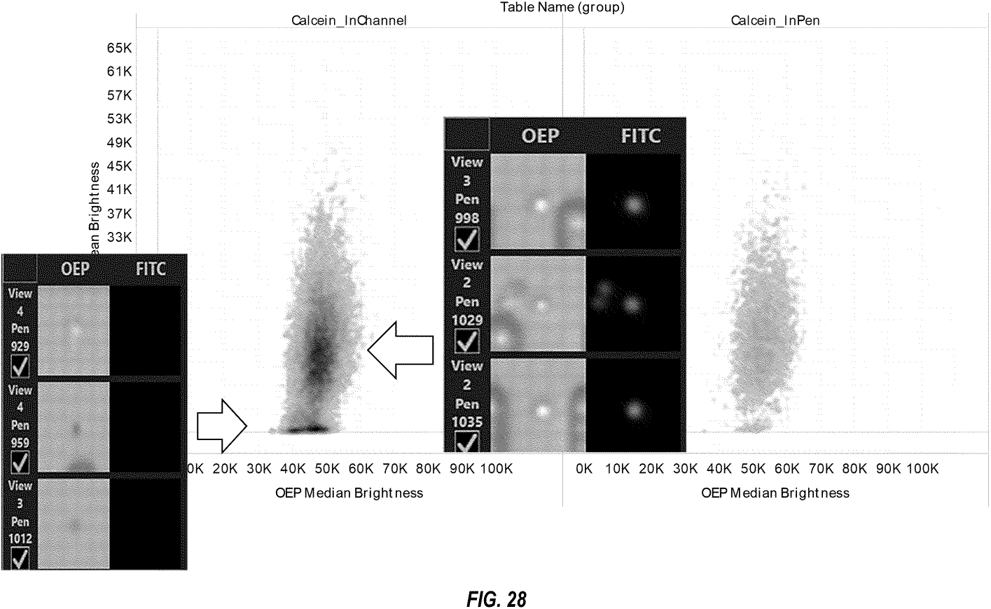

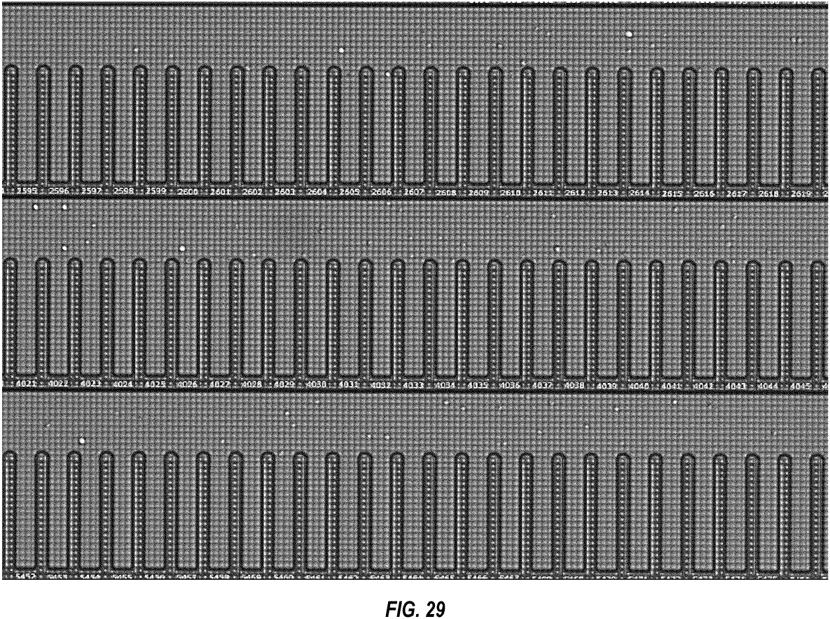

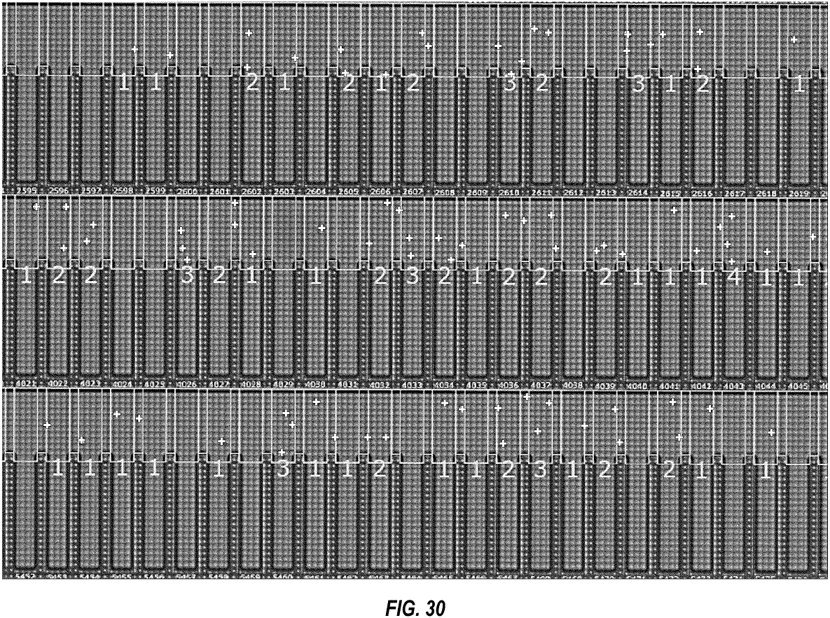

It was then attempted to determine whether the live-stain and brightfield images of plasma cells in Beacon system have good association with each other. In brightfield image sequences, cells with different morphologies (FIG. 27) were seen. Investigation was performed to determine whether these differences in morphologies were relatable with the live cell stain (calcein). With the dataset of cells stained with calcein (FIG. 28), it was validated that cells with the low calcein fluorescence were associated with cells with non-clear boundaries and smaller cell diameter. Fresh samples (a pool of 3 chips): each dot denoted a cell. Although OEP median brightness (and also other TPS (Target and Pen Selection) parameters) is not particularly effective in distinguishing live cells from dead cells, it can be visually observed that live cells have clear outlines, while dead cells have unclear outlines.

In summary, It was demonstrated that that Beacon system can be used to assess live and dead cells based on cell stains (calcein, Zombie). Furthermore, the live-stain is associated with the brightfield images and matches with off-chip flow cytometry data, proving it is a reliable source for training a convolutional neural network.

B. Training of the Convolutional Neural Network. A CNN B cell live/dead classification model was trained and established. The B cell live/dead classification model can be an additional neural network feature that utilizes the output from a B cell detection model of the CNN. Since the live / dead classification model is a separate module from the B cell detection model, the live/dead classification model can be turned on and off without affecting cell detection. Once B cells are detected, new cell images are produced based on centroid locations of cells and are passed into the live/dead classification model. The output of this classification model is a probability of the cell being live.

Training the model: Training data consisted of cells stained with calcein using FITC dye, in combination with images of the cells under OEP (brightfield). Cells were first detected using the B cell detection model under OEP. Afterwards, cells were labeled as live / dead based on fluorescent intensity under the FITC fluorescent cube (via TPS). A classification model was then trained using a combination of cell images under OEP and the labels gathered using the FITC dye (see Generating Input Data and Generating Labels paragraphs below). Table 6 shows six different microfluidic chips were utilized for training a live / dead cell classification model.

TABLE 6

| | | | | |
|---|---|---|---|---|
| Nest # | Device ID | Experiment | Tool ID | Script Revision |
| Nest 1 | D71954 | Fresh Cells: Mouse plasma cells with calcein(live), Zombie (dead), Annexin (dead) staining. IgG bead capture assay afterwards | BSN0025 | CAS1.5 |
| Nest 2 | D71956 | | BSN0025 | CAS1.5 |
| Nest 3 | D71977 | | BSN0025 | CAS1.5 |
| Nest 1 | D71961 | Frozen/Thawed Cells: Mouse plasma cells with calcein (live), Zombie (dead), Annexin (dead) staining. IgG bead capture assay afterwards | BSN0025 | CAS1.5 |
| Nest 2 | D71967 | | BSN0025 | CAS1.5 |
| Nest 3 | D73451 | | BSN0025 | CAS1.5 |

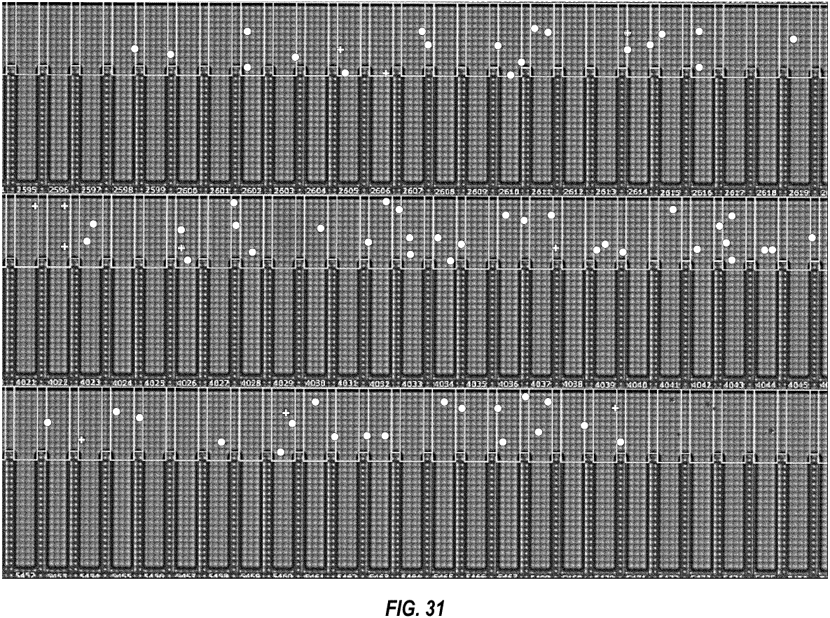

FIG. 29, FIG. 30, FIG. 31 were obtained from the same image, and demonstrated how training data was generated. FIG. 29 shows raw data used for training, with an OEP (brightfield) and a FITC (calcein) channel overlay. Green glowing cells denoted a cell stained with calcein, whereas other cells did not have this stain.

Generating Input Data. FIG. 30 shows cells detected using the B cell detection model on FIG. 29 under brightfield. Each cell was used as the input to the live/dead classification model. Each detected B cell is denoted with a ‘+’. The vertical lines divide the channel into segments, each corresponding to a destination pen. The number indicated at the opening of each pen represents the number of B cells detected in each segment of the channel.

Generating Labels for live/dead cells. Live/dead cell labels for detected B cells from FIG. 31 were gathered based on fluorescent intensities via TPS, using a cutoff based on the FITC channel’s Mean Brightness value of ~10000 (16 bit unsigned int). A solid circle denoted a live cell label. A ‘+’ denoted a dead cell label. FIG. 31 was used as the expected output for the live/dead classification model.

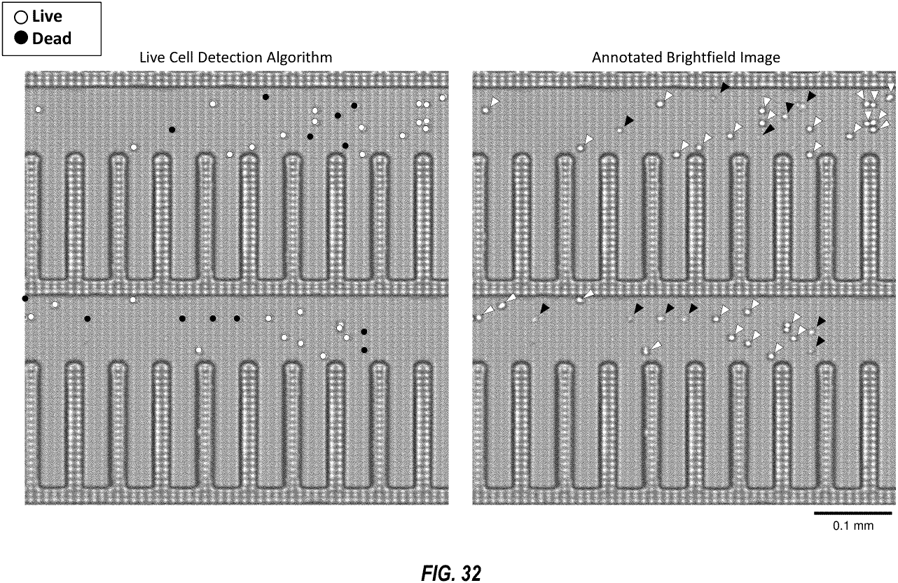

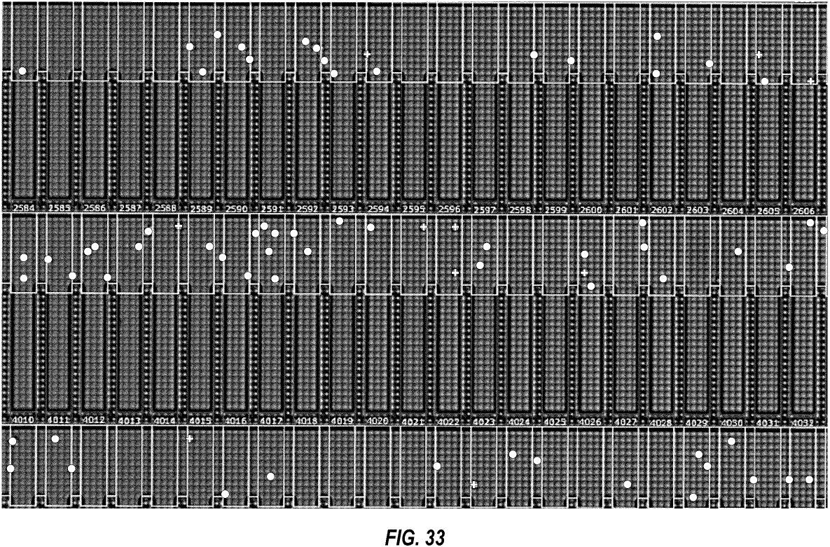

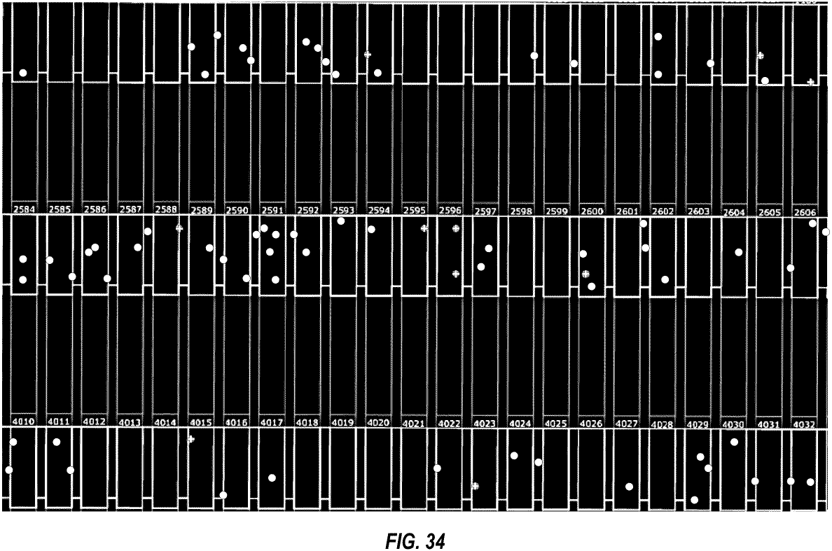

The trained live/dead classification model was used in a stain-free sample to identify live B cells from dead B cells. The result is shown in FIG. 32. The image in left shows the live cells (in solid white) and dead cells (in solid back) recognized by the algorithm. The image in right is a brightfield image annotated by human eyes verifying the algorithm was accurate.

Qualitative Metrics: Six different devices as shown in Table 7 are utilized for evaluation. FIG. 33 and FIG. 34 were obtained from the same image, and demonstrated how evaluation data was analyzed. These images demonstrate that the model properly classified detected B cells as live/dead based on only an OEP image. This was validated by using the calcein stain (FITC channel) to denote where the true live cells were present.

TABLE 7

| Nest # | Device ID | Experiment | Tool ID | Script Revision |
|---|---|---|---|---|
| Nest 1 | D73449 | | BSN0025 | 1.5 |
| Nest 2 | D73720 | Fresh Cells with calcein: Pen FOV 0-13: with calcein stain gating; Pen FOV 14-27: without calcein stain gating. IgG bead capture assay afterwards | BSN0025 | 1.5 |
| Nest 3 | D74778 | Frozen/Thawed Cells with calcein: Pen FOV 0-13: with calcein stain gating; Pen FOV 14-27: without calcein stain gating. IgG bead capture assay afterwards | BSN0025 | 1.5 |
| Nest 4 | D74779 | | BSN0025 | 1.5 |
| Nest 3 | D73474 | Cells with calcein (poor quality cells from a well are added to decrease cell viability): Pen FOV 0-13: with calcein stain gating; Pen FOV 14-27: without calcein stain gating. IgG bead capture assay afterwards | BSN0025 | 1.5 |
| Nest 4 | D73722 | | BSN0025 | 1.5 |

Evaluation data, OEP and FITC (calcein) channel overlay. FIG. 33 shows the output of unseen evaluation data (withheld from training a model, to avoid biasing the output). All B cells detected from the B cell detection model (under OEP) were labeled with a ‘+’ (aqua and red). The green glowing B cells (denoting calcein stain) with a solid circle were predicted as live cells. Cell with a ‘+’ were predicted as dead. These cells have no calcein staining since they do not glow green.

Evaluation data, FITC (calcein) channel only. FIG. 34 is the same as above, but with the OEP channel turned off (no brightfield) to provide another view of the same information. All cells detected from the B cell detection model (under OEP) were labeled with a ‘+’ (aqua and red). The green glowing B cells (denoting calcein stain) with a solid circle were predicted as live cells. Cell with a ‘+’ were predicted as dead and cannot be seen under the FITC cube due to lack of calcein staining.

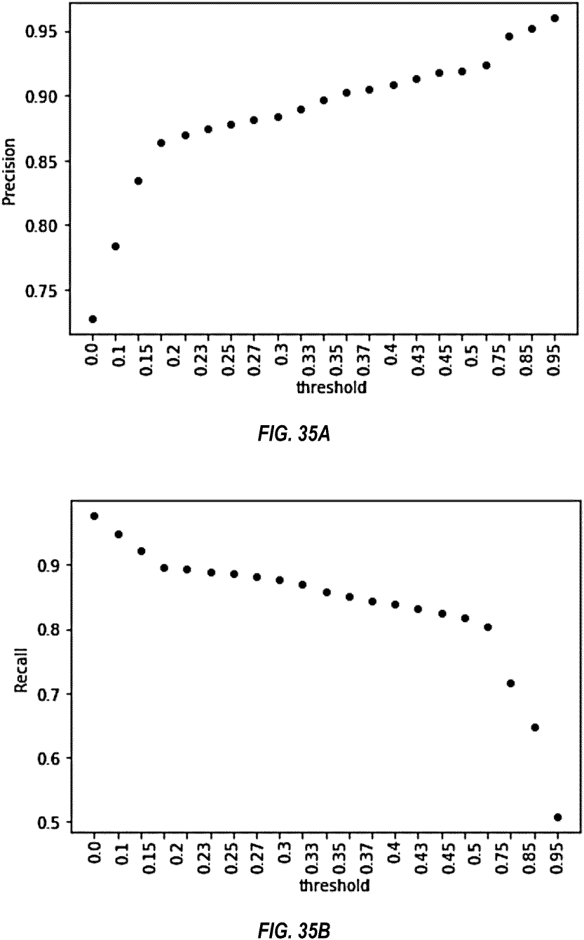

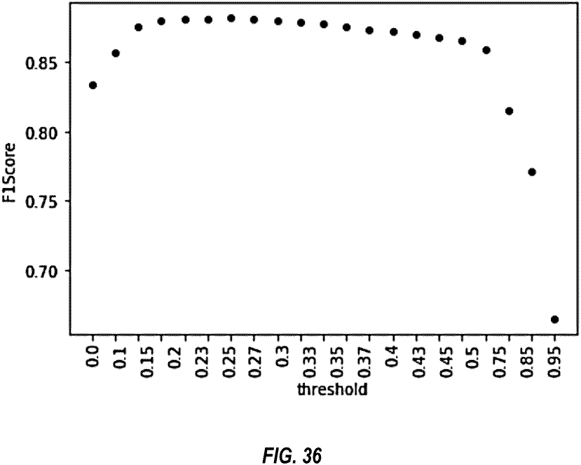

Quantitative Metrics: The following plots provide insight into quantitative metrics for experiment D74779. A threshold set to 0 is the same as turning this live / dead classification feature off. Tuning a proper cutoff can be done by the user to their likings that trades off precision and recall of live/dead cells. As shown in FIGS. 35A-35B, setting a higher threshold cutoff will increase the percentage of truly live cells (increased precision), at the cost of the number of total live cells retrieved (decreased recall). The F1 score (FIG. 36) is a measure of a test’s accuracy, which is the harmonic mean between precision and recall.

### Example 5 On-Chip Lysis, RNA Capture, Label Detection and Export

System, in-pen assay reagents, and cells are similar to the materials in Example 1. Labelled and barcoded nucleic acid capture beads included 12 sets of differently labelled (e.g., detectably distinguishable labelled), barcoded nucleic acid capture objects as described herein. Each set of detectably distinguishable nucleic acid capture beads has an integral bead color (commercially available from Spherotech) that is distinct from any of the other eleven sets of nucleic acid capture beads. Further, each capture bead of each set of detectably distinguishable nucleic acid capture beads includes a barcode sequence (e.g. oligonucleotide sequence) that is paired to that specific integral bead color. The label and the barcode sequence are each the same for each nucleic acid capture bead of each set of detectably distinguishable nucleic acid capture beads. The twelve distinct barcode sequences are the sequences shown in SEQ ID NOs: 1-12 in Table 8.

Label Detection. Bead type/barcode detection used a maximum entropy classification model with stochastic dual coordinate ascent (SDCA). This model used an input of the normalized fluorescent intensity based on 4 filter cubes (scale from 0 to 1.0): Cy5, DAPI, FITC, TRED, and output a probability of the bead belonging to a particular bead barcode (e.g. C0D0F0T1, where C denotes Cy5, D denotes DAPI, F denotes FITC, T denotes TRED; 0 and 1 are on and off binary numbers). During training, the model used the same input features as described above (Cy5, DAPI, FITC, TRED), and an expected bead barcode output ground truth was provided based on bead import data. The ground truth dataset was created by importing each bead type from a well plate via an export/import needle on the instrument controlling the microfluidic chip. Each bead type was penned to specific fields of view and assigned to a specific pen ID. Bead types were spatially separated across the fields of views in a chip. The ground truth dataset of pen ID, field of view number, and fluorescent images of all cubes were used to train and test the accuracy of the bead classification model.

Cells were imported into the microfluidic device, and individual cells were imported using DEP forces into individual sequestration pens. Individual healthy cells were selected for penning based on the trained CNN methods described above in Example 2, but penning can be accomplished in other manners, such as manual penning, cell staining followed by selective importation, and bulk penning. Antibody binding/functional assays were performed as described in Example 1.

On chip lysis, nucleic acid capture and RT. After completion of assays designed to detect antibodies secreted by the cells, the plurality of 12 distinct sets of labelled, barcoded nucleic acid capture beads, described in the previous paragraph, were imported into the flow channel of the microfluidic device. The labelled barcoded nucleic acid beads were imported into sequestration pens containing a single cell or single clonal population, to deliver one labelled barcode nucleic acid bead per sequestration pen. The process of importation of the labelled barcoded beads into the sequestration pen included the use of DEP forces to select a desired labelled barcoded bead for each sequestration pen. Typically, but not required, importation of the labelled barcoded beads was performed to import a different color, and hence, barcode, for a set of adjacent sequestration pens.

After importation of the distinguishably labelled, barcoded nucleic acid capture beads, on-chip cell lysis was then performed by importing a lysis reagent including a detergent-based cell lysis buffer (24 microliters); PBS including magnesium, calcium chloride, F127, and RNase inhibitor (31.8 microliters); PEG 4000 (1.2 microliter) and RNase OUT™ (3 microliter, Invitrogen), at a perfusion rate of 0.1 microliter/sec. The lysis reagent diffused into the sequestration pens, and the cells were exposed to lysis reagent for 10 min at 25° C. The microfluidic chip was then flushed with a wash buffer including saline sodium citrate buffer. During the lysing and flush period, RNA from the lysed cells was captured to the nucleic acid capture object within that individual pen.

On-chip reverse transcription was performed by lowering the temperature of the microfluidic device to 16° C. Reverse transcription reagent (15 microliters), including water; 5x RT Buffer; dNTPs; PEG 4000; and RT enzyme was imported onto the chip, and the reagent diffused into the sequestration pens. On-chip reverse transcription was performed by cycling the microfluidic chip temperature as follows: 10 min at 20° C.; 10 min at 30° C.; 90 min at 42° C.; 10 min at 30° C.; and 10 min at 20° C. The chip was then cooled to 18° C. for bead classification and subsequent export.

For bead barcode/type detection, the beads were imaged in multiple fluorescent channels using the maximum entropy classification model with stochastic dual coordinate ascent (SDCA) described above. The identity of the label was stored with the identity of the sequestration pen. This permitted correlation of the antibody binding/functional assay results for that pen to the nucleic acid capture object imported there, allowing for correlation of binding/functional assays to sequencing results for the cell/clonal population in that sequestration pen.

While in this experiment determination of the identity of the label of the barcoded nucleic acid capture object was performed after reverse transcription of the captured RNA, detection of the label may be performed at other points during any of the processes performed while the nucleic acid capture object is disposed within the sequestration pen.

After nucleic acid capture object classification, beads were selected for export. Exports were performed by selecting one bead of each color type, e.g., one of each distinct set of labelled, barcoded nucleic acid capture objects, and exporting each set of twelve differently labelled capture objects to a single well in a 96 well plate. This was repeated sequentially across the chip. Up to 1152 exports of target BCR sequences were achieved by exporting 12 distinct types of beads as one batch per well.

The exported beads with cDNA were processed for downstream sequencing and, optionally, re-expression as described in Example 1.

### Example 6 Demultiplexing Barcoded Export cDNA

Amplification of a specific antibody variable domain from barcoded export cDNA may be accomplished by preparing a PCR with a single barcoded forward primer matching the desired barcode and a common reverse primer designed to bind to either the Hc or Lc constant region.

Barcoded heavy chain and light chain variable domain amplicons from pooled plasma cell export cDNA were amplified using the KAPA HiFi HotStart ReadyMix. Barcoded amplicons were amplified in independent reactions using barcode specific forward primers, and common reverse primers targeting either the heavy or light constant domains.

The PCR was run with the following conditions:
98° C. 3 min; followed by:
   24 cycles including 98° C. for 20 sec; 70° C. for 15 sec; 72° C. for 45 sec.
After completing the 24 cycles of PCR, the reaction was incubated at 72° C. for a further 3 min; Final Hold at 4° C.

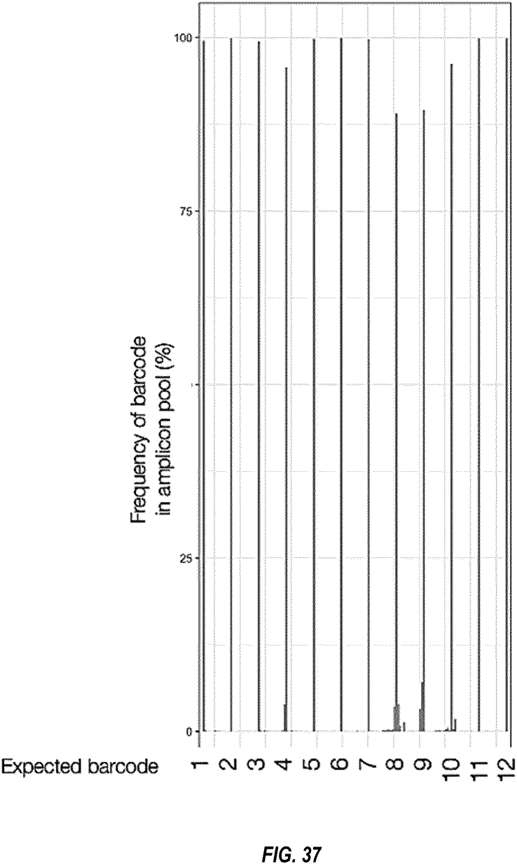

The frequency of amplicons with the expected barcode from PCR reactions using barcode specific forward primers as determined by NGS sequencing is shown in FIG. 37. A series of 12 histograms, one for each expected barcode, depicting the amount of the observed barcodes on amplicons from export cDNA amplified using barcode-specific primers. Template from 12 export wells, each containing cDNA from 12 barcoded-bead exports were amplified as described using barcode specific primers. These variable domain amplicons were indexed and sequenced using standard NGS library using standard protocols. The fraction of reads containing all barcodes, both expected and unexpected, was analyzed for each sample to determine the specificity of barcode primer amplification. Some small fraction of reads for non-expected barcodes in some samples were observed. The original histograms were in color to differentiate frequencies of 12 different barcodes and the black and white version of the histograms are shown in FIG. 37. For example, the histogram shows a small fraction of reads for different barcodes (as represented by bars centered about the major bar) for barcode 8, none of which have a frequency greater than a few percent of the correct barcode read. This demonstrates that there is some non-correct amplification when the bead having barcode 8 is amplified. Amplicons with the expected barcode 8 represent more than 87.5% of reads, demonstrating the specificity of amplification for each barcoded set of cDNA captured from the on-chip lysis and RNA capture. Barcode 9 shows fewer cross-amplification events but one specific non-correct read is present up to about 5% of the total reads, and the expected barcode 9 is present in more than about 88% of the reads captured using beads having barcode 9. Barcode 10 and barcode 4 show very small incidences of cross-reads of incorrect barcoded sequences, providing greater than about 95% of the expected barcode 10 or barcode 4 in reads from the barcoded set of cDNA captured from the respective set of beads having barcode 10 and barcode 4.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner. Furthermore, where reference is made herein to a list of elements (e.g., elements a, b, c), such reference is intended to include any one of the listed elements by itself, any combination of less than all of the listed elements, and/or a combination of all of the listed elements. Also, as used herein, the terms a, an, and one may each be interchangeable with the terms at least one and one or more. It should also be noted, that while the term step is used herein, that term may be used to simply draw attention to different portions of the described methods and is not meant to delineate a starting point or a stopping point for any portion of the methods, or to be limiting in any other way.

### XIV. Additional Embodiments

Embodiment 1. A method of assaying for inhibition of a specific binding interaction between a first molecule and a second molecule, wherein the method is performed within a microfluidic device having a chamber, the method comprising: introducing a micro-object into the chamber of the microfluidic device, wherein the micro-object comprises a plurality of first molecules; introducing a cell into the chamber, wherein the cell is capable of producing a molecule of interest; incubating the cell in the chamber, in the presence of the micro-object, and under conditions conducive to production and secretion of the molecule of interest; after incubating the cell in the chamber, introducing the second molecule into the chamber, wherein the second molecule is bound to a detectable label; and monitoring an accumulation of the second molecule on the micro-object, wherein an absence or diminishment of accumulation of the second molecule on the micro-object indicates that the molecule of interest inhibits binding of the first molecule to the second molecule.

Embodiment 2: The method of embodiment 1, wherein the molecule of interest binds to first molecules on the micro-object and thereby inhibits binding of the second molecule to the micro-object.

Embodiment 3. The method of embodiment 1, wherein the molecule of interest binds to second molecules and thereby inhibits binding of the second molecules to the first molecules on the micro-object.

Embodiment 4. The method of any one of embodiments 1 to 3, wherein the first molecule is a receptor molecule, and wherein the second molecule is a ligand that specifically binds to the receptor molecule.

Embodiment 5. The method of any one of embodiments 1 to 3, wherein the first molecule is a ligand, and wherein the second molecule is a receptor that is specifically bound by the ligand.

Embodiment 6. The method of embodiment 4 or 5, wherein the receptor molecule is a protein, and, optionally, a glycosylated protein.

Embodiment 7. The method of embodiment 6, wherein the receptor is a growth factor receptor, a cytokine receptor, a chemokine receptor, an adhesion receptor (e.g., an integrin or a cell adhesion molecule (CAM)), an ion channel, a G protein-coupled receptor (GPCR), or a fragment retaining activity of its respective full length biomolecule of any of the foregoing.

Embodiment 8. The method of any one of embodiments 4 to 7, wherein the ligand is a protein.

Embodiment 9. The method of any one of embodiments 4 to 8, wherein the ligand is a growth factor, a cytokine, a chemokine, an adhesive ligand, an ion channel ligand, a GPCR ligand, a viral protein (e.g., a viral fusion protein), or a fragment retaining activity of its respective full length biomolecule of any of the foregoing.

Embodiment 10. The method of any one of embodiments 1 to 9, wherein the micro-object comprising the plurality of first molecules is a cell.

Embodiment 11. The method of embodiment 10, wherein the cell comprising the plurality of first molecules is from a transfected cell line (e.g., stably or transiently transfected).

Embodiment 12. The method of embodiment 11, wherein at least 60% (e.g., at least 65%, at least 70%, at least 75%, at least 80%, at least 85%, at least 90%, at least 95%, or more) of the cells in the transfected cell line express the first molecules at detectable levels.

Embodiment 13. The method of any one of embodiments 10 to 12, wherein the cell that comprises the plurality of first molecules comprises an exogenous nucleic acid molecule encoding the first molecule.

Embodiment 14. The method of any one of embodiments 1 to 13, wherein the plurality of first molecules comprised by the micro-object is sufficient to bind at least 50,000 second molecules (e.g., at least 60,000, at least 70,000, at least 80,000, at least 90,000, at least 100,000, at least 110,000, at least 120,000, at least 130,000, at least 140,000, at least 150,000, or more second molecules).

Embodiment 15. The method of any one of embodiments 1 to 14, wherein the molecule of interest is an antibody.

Embodiment 16. The method of embodiment 15, wherein the cell capable of producing the molecule of interest is an antibody producing cell (APC).

Embodiment 17. The method of embodiment 15, wherein the cell capable of producing the molecule of interest is a B cell, and, optionally, a plasma cell.

Embodiment 18. The method of embodiment 15, wherein the cell capable of producing the molecule of interest is a memory B cell and, optionally, wherein incubating the cell capable of producing the molecule of interest under conditions conducive to production and secretion of the molecule of interest comprises contact the cell capable of producing the molecule of interest with one or more memory B cell activating agents.

Embodiment 19. The method of any one of embodiments 1 to 18, wherein introducing the micro-object into the chamber of the microfluidic device comprises introducing a single micro-object into the chamber of the microfluidic device.

Embodiment 20. The method of embodiment 19, wherein the single micro-object is selectively introduced into the chamber, optionally using dielectrophoresis (DEP) force.

Embodiment 21. The method of any one of embodiments 1 to 18, wherein introducing the micro-object into the chamber of the microfluidic device comprises introducing a plurality of micro-objects into the chamber of the microfluidic device.

Embodiment 22. The method of embodiment 21, wherein introducing the plurality of micro-objects into the chamber of the microfluidic device comprises introducing three, four, or five micro-objects into the chamber of the microfluidic device.

Embodiment 23. The method of embodiment 21 or 22, wherein the plurality of micro-objects are introduced into the chamber using DEP force or gravity.

Embodiment 24. The method of any one of embodiments 1-23, wherein introducing the micro-object into the chamber comprises selectively introducing the micro-object based on detecting a condition of viability for the micro-object, optionally using dielectrophoresis (DEP) force.

Embodiment 25. The method of embodiment 24, wherein detecting the condition of viability further comprises employing a machine-learning algorithm to assign a probability of viability to the single micro-object or the plurality of micro-objects.

Embodiment 26. The method of embodiment 25, wherein the machine-learning algorithm comprises a trained machine-learning algorithm, wherein the trained machine-learning algorithm comprises training a machine-learning algorithm by imaging micro-objects comprising a label demarking a condition of viability.

Embodiment 27. The method of embodiment 26, wherein the micro-objects comprising the label demarking viability are a same type of cells as the single micro-object or the plurality of micro-objects to be selected for introduction to the chamber or plurality of chambers.

Embodiment 28. The method of embodiment 26 or 27, wherein the label demarking viability comprises a live/dead stain comprising calcein, zombie violet stain, annexin, acridine orange, propidium iodide, or any combination thereof.

Embodiment 29. The method of any one of embodiments 26 to 28, wherein the training further comprises imaging the micro-objects comprising the label demarking viability under brightfield conditions.

Embodiment 30. The method of any one of embodiments 1 to 29, wherein the chamber is a microwell.

Embodiment 31. The method of any one of embodiments 1 to 29, wherein the chamber is a sequestration pen.

Embodiment 32. The method of embodiment 31, wherein the microfluidic device comprises a microfluidic channel, wherein the sequestration pen comprises an isolation region and a connection region, and wherein the connection region has a proximal opening to the microfluidic channel and a distal opening to the isolation region.

Embodiment 33. The method of embodiment 32, wherein the isolation region comprises a single opening to the connection region.

Embodiment 34. The method of embodiment 32 or 33, wherein the sequestration pen has a single opening to the microfluidic channel.

Embodiment 35. The method of any one of embodiments 1 to 34, wherein the chamber comprises a volume of about 200 pL to about 10 nL (e.g., about 200 pL to about 5 nL, or about 250 pL to about 2 nL).

Embodiment 36. The method of any one of embodiments 1 to 35, wherein introducing the second molecule into the chamber comprises flowing a medium comprising the second molecule into a microfluidic channel which is fluidically connected to the chamber and allowing the second molecule to diffuse into the chamber.

Embodiment 37. The method of any one of embodiments 1 to 36, wherein the microfluidic device comprises a plurality of chambers, and wherein the method further comprises: introducing a micro-object into each chamber of the plurality of chambers, wherein the micro-object comprises a plurality of first molecules; introducing a cell into each chamber of the plurality of chambers, wherein the cell is capable of producing a molecule of interest; incubating the cells in the plurality of chambers, in the presence of the micro-objects, and under conditions conducive to production and secretion of the molecule of interest; after incubating the cells in the plurality of chambers, introducing the second molecule into each chamber of the plurality of chambers, wherein the second molecule is bound to a detectable label; and monitoring an accumulation of the second molecule on the micro-objects.

Embodiment 38. The method of any one of embodiments 1-37, wherein monitoring an accumulation of the second molecule on each of the micro-objects comprises comparing the accumulation to that observed in the presence of a positive control molecule of interest and/or a negative control molecule of interest.

Embodiment 39. A method of providing one or more barcoded cDNA sequences from a biological cell, comprising: providing the biological cell within a chamber; providing a capture object in the chamber, the capture object comprising a label, a plurality of first oligonucleotides, and a plurality of second oligonucleotides, wherein each first oligonucleotide of the plurality comprises a barcode sequence, and a sequence comprising at least three consecutive guanine nucleotides at a 3′ end of each first oligonucleotide, wherein each second oligonucleotide of the plurality comprises a capture sequence, lysing the biological cell and allowing RNA released from the lysed biological cell to be captured by the capture sequences of the plurality of second oligonucleotides, thereby forming captured RNA; and reverse transcribing the captured RNA, thereby producing one or more barcoded cDNA sequences, each comprising an oligonucleotide sequence complementary to a corresponding one of the captured RNA and covalently linked to the reverse complement of the barcode sequence of the first oligonucleotide.

Embodiment 40. The method of embodiment 39, wherein the chamber comprises a microwell.

Embodiment 41. The method of embodiment 39, wherein the chamber comprises a sequestration pen of a microfluidic device.

Embodiment 42. The method of any one of embodiments 39 to 41, wherein a single capture object is provided in the chamber.

Embodiment 43. The method of any one of embodiments 39 to 42, wherein the first oligonucleotide comprises a first priming sequence that corresponds to a first primer sequence and/or wherein the second oligonucleotide comprises a second priming sequence that corresponds to a second primer sequence.

Embodiment 44. The method of embodiment 43, wherein the first and second primer sequences are the same.

Embodiment 45. The method of any one of embodiments 39 to 44, wherein the capture sequence binds to, and thereby, captures RNA and primes transcription from the captured RNA.

Embodiment 46. The method of embodiment 45, wherein a reverse transcription (RT) polymerase transcribes captured RNA.

Embodiment 47. The method of any one of embodiments 39 to 46, wherein the barcode sequence of the first oligonucleotide corresponds to the label of the capture object.

Embodiment 48. The method of embodiment 47, wherein the label is an integral color of the capture object.

Embodiment 49. The method of any one of embodiments 39 to 46, wherein the barcode sequence of the first oligonucleotide is the label of the capture object.

Embodiment 50. The method of any one of embodiments 39 to 49, further comprising identifying the barcode sequence of the plurality of first oligonucleotides while the capture object is located within the chamber.

Embodiment 51. The method of embodiment 50, wherein identifying the barcode comprises detecting fluorescence emitted from the label.

Embodiment 52. The method of any one of embodiments 39 to 51, wherein the label comprises one or more fluorophores.

Embodiment 53. The method of embodiment 52, wherein the label comprises a single fluorophore.

Embodiment 54. The method of embodiment 52, wherein the label comprises multiple fluorophores.

Embodiment 55. The method of any one of embodiments 39 to 54, wherein the first oligonucleotide comprises one or more uridine nucleotides 5′ to the barcode sequence and, if present, the first priming sequence.

Embodiment 56. The method of any one of embodiments 39 to 54, wherein the first oligonucleotide comprises three uridine nucleotides 5′ to the barcode sequence and, if present, the first priming sequence.

Embodiment 57. The method of embodiment 55 or 56, wherein the one or more uridine nucleotides are adjacent to or comprise the 5′-most nucleotide(s) of the first oligonucleotide.

Embodiment 58. The method of any one of embodiments 39 to 57, wherein reverse transcribing the captured RNA is performed in the presence of an enzyme that cleaves a sequence containing one or more uridine nucleotides (e.g., a USER enzyme).

Embodiment 59. The method of any one of embodiments 39 to 58, wherein the first oligonucleotide comprises three guanine nucleotides at a 3′ end.

Embodiment 60. The method of any one of embodiments 39 to 59, wherein the capture sequence of the second oligonucleotide of the plurality of capture objects comprises an oligo-dT sequence (e.g., a (T)x VN sequence or an (T)x VI sequence, wherein X is greater than 10, 15, 20, 25, or 30).

Embodiment 61. The method of any one of embodiments 39 to 60, wherein the ratio of the second oligonucleotide to the first oligonucleotide on the capture object ranges from 1:10 to 10:1.

Embodiment 62. The method of any one of embodiments 39 to 61, wherein the ratio of the second oligonucleotide to the first oligonucleotide on the capture object is about 1:1 (e.g., 95:100 to 100:95).

Embodiment 63. The method of any one of embodiments 39 to 62, wherein the first oligonucleotide comprises, consists of, or consists essentially of RNA.

Embodiment 64. The method of any one of embodiments 39 to 63, wherein the first oligonucleotide comprises at least one modified base.

Embodiment 65. The method of embodiment 64, where the at least one modified base independently comprises a 2′-O-methyl base, O-methoxy-ethyl (MOE) base, or a locked nucleic acid base.

Embodiment 66. The method of any one of embodiments 39 to 65, wherein the first oligonucleotide comprises at least one phosphorothioate bond.

Embodiment 67. The method of any one of embodiments 39 to 66, wherein the first oligonucleotide is linked to the capture object.

Embodiment 68. The method of any one of embodiments 39 to 66, wherein the first oligonucleotide is covalently bound to the capture object.

Embodiment 69. The method of embodiment 68, wherein the first oligonucleotide is linked to the capture object by streptavidin-biotin binding.

Embodiment 70. The method of any one of embodiments 39 to 69, wherein the second oligonucleotide is linked to the capture object.

Embodiment 71. The method of any one of embodiments 39 to 69, wherein the second oligonucleotide is covalently bound to the capture object.

Embodiment 72. The method of embodiment 70, wherein the second oligonucleotide is linked to the capture object by streptavidin-biotin binding.

Embodiment 73. The method of any one of embodiments 39 to 72, wherein each of the one or more barcoded cDNA sequences is associated with the capture object.

Embodiment 74. The method of any one of embodiments 39 to 73, wherein the one or more barcoded cDNA sequences are produced in the chamber.

Embodiment 75. The method of any one of embodiments 39 to 74, further comprising exporting the capture object from the chamber.

Embodiment 76. The method of any one of embodiments 39 to 75, further comprising storing the one or more barcoded cDNA sequences.

Embodiment 77. The method of any one of embodiments 39 to 76, wherein the one or more barcoded cDNA sequences are stored at a temperature of about 4° C.

Embodiment 78. The method of any one of embodiments 39 to 77, further comprising amplifying the one or more barcoded cDNA sequences.

Embodiment 79. The method of the embodiment 78, wherein amplifying the one or more barcoded cDNA sequences comprises using a single primer (e.g., a P1 primer).

Embodiment 80. The method of any one of embodiments 39 to 79, further comprising performing the method on a plurality of biological cells provided in a corresponding plurality of chambers.

Embodiment 81. The method of embodiment 80, wherein a plurality of capture objects are provided to the plurality of chambers, each capture object of the plurality having (i) a unique label selected from a plurality of unique labels (e.g., at least 12, 14, 16, 18, 20, 25, 30, 40, 50, 60, 70, 80, 90, 100, 150, 200, 250, 500, 1000, or more different labels), and (ii) a plurality of first oligonucleotides having a barcode sequence corresponding to the unique label.

Embodiment 82. The method of embodiment 81, further comprising: exporting the plurality of capture objects into a common receptacle; and amplifying the one or more barcoded cDNA sequences from each capture object of the plurality, thereby producing a plurality of barcoded cDNA sequences, each barcoded cDNA sequence having a barcode sequence corresponding to one of the plurality of unique labels.

Embodiment 83. The method of any one of embodiments 39 to 82, wherein providing one or more barcoded cDNA sequences comprises providing a plurality of barcoded cDNA sequences, each barcoded cDNA sequence of the plurality encoding a protein of interest, corresponding to any one of a plurality of different proteins, linked to a corresponding reverse complement barcode sequence; and the method further comprising: optionally amplifying the plurality of barcoded cDNA sequences; selectively amplifying the plurality of barcoded cDNA sequences (or amplified cDNA sequences) using a barcode-specific forward primer and a reverse primer specific to the protein of interest to produce an amplified cDNA product (or further amplified cDNA product) encoding the protein of interest or a fragment thereof; annealing a 5′ end of the amplified cDNA product (or further amplified cDNA product) to a 5′ corresponding end of a DNA fragment for transcriptionally-active PCR (TAP) to produce an annealed TAP product; and amplifying the annealed TAP product via overlap extension PCR using a TAP adapter primer to produce a construct for expression of the protein of interest.

Embodiment 84. The method of embodiment 83, wherein the reverse primer specific to the protein of interest comprises a sequence complementary to a sequence encoding a conserved region (e.g., a constant portion) of the protein of interest, or a sequence 3′ to the conserved region (e.g., a 3′ UTR sequence).

Embodiment 85. The method of embodiment 83 or 84, wherein a 3′ end of the amplified cDNA product (or further amplified cDNA product) comprises a region overlapping with a 3′ corresponding end of the DNA fragment for TAP.

Embodiment 86. The method of any one of embodiments 39 to 82, wherein providing one or more barcoded cDNA sequences comprises providing a plurality of barcoded cDNA sequences, each barcoded cDNA sequence of the plurality encoding a heavy chain or a light chain sequence corresponding to any one of a plurality of different antibodies, linked to a corresponding reverse complement barcode sequence; the method further comprising: optionally amplifying the plurality of barcoded cDNA sequences; selectively amplifying the plurality of barcoded cDNA sequences using a barcode-specific forward primer and a reverse primer targeting a conserved portion of the corresponding constant region sequence (e.g., a 5′ end, or sequence adjacent thereto, of the constant region) to produce an amplified cDNA product (or further amplified cDNA product) encoding the barcode-specific variable region; annealing ends of the amplified cDNA product (or further amplified cDNA product) to corresponding ends of a DNA fragment for TAP to produce an annealed TAP product; and amplifying the annealed TAP product via overlap extension PCR using TAP adapter primers to produce an expression construct for expression of an antibody heavy chain or light chain.

Embodiment 87. The method of any one of embodiments 83-86, wherein amplifying the plurality of barcoded cDNA sequences comprises using a single primer (e.g., a P1 primer).

Embodiment 88. The method of any one of embodiments 83-87, wherein amplifying the plurality of barcoded cDNA sequences comprises using different forward and reverse primers.

Embodiment 89. The method of any one of embodiments 39 to 82, wherein providing one or more barcoded cDNA sequence comprises providing a mixture of barcoded cDNA sequences, each barcoded cDNA sequence of the mixture encoding a heavy chain or a light chain sequence, corresponding to any one of a plurality of different antibodies, linked to a corresponding reverse complement barcode sequence.

Embodiment 90. The method of any one of embodiments 39 to 82, wherein the method comprises: providing a first barcoded cDNA sequence, comprising a nucleic acid encoding a heavy chain of an antibody, linked to a reverse complement of a first barcode sequence at a 5′ end; and providing a second barcoded cDNA sequence, comprising a nucleic acid encoding a light chain of the same antibody, linked to a reverse complement of a second barcode sequence at a 5′ end.

Embodiment 91. The method of embodiment 90, wherein the first and second barcode sequences are the same.

Embodiment 92. The method of embodiment 90, wherein the first and second barcode sequences are different.

Embodiment 93. The method of any one of embodiments 90 to 92, wherein the method comprises: providing a first DNA fragment for transcriptionally active PCR (TAP), the DNA fragment comprising: a promoter sequence, a constant domain sequence 3′ to a respective variable region of the heavy chain of the antibody, and a terminator sequence; providing a second DNA fragment for transcriptionally active PCR (TAP), the DNA fragment comprising: a promoter sequence, a constant domain sequence 3′ to a respective variable region of the light chain of the antibody, and a terminator sequence.

Embodiment 94. The method of any one of embodiments 90 to 93, wherein the method comprises: providing a first barcoded cDNA sequence, comprising a nucleic acid encoding a heavy chain of an antibody, linked to a first barcode sequence at a 5′ end; providing a second barcoded cDNA sequence, comprising a nucleic acid encoding a light chain of the same antibody, linked to a second barcode sequence at a 5′ end; amplifying at least a portion of the first barcoded cDNA sequence using a first barcode-specific primer; amplifying at least a portion of the second barcoded cDNA sequence using a second barcode-specific primer; providing a first DNA fragment for transcriptionally active PCR (TAP), the DNA fragment comprising: a promoter sequence, a constant domain sequence 3′ to a respective variable region of the heavy chain, and a terminator sequence; providing a second DNA fragment for transcriptionally active PCR (TAP), the DNA fragment comprising: a promoter sequence, a constant domain sequence 3′ to a respective variable region of the light chain, and a terminator sequence; incorporating the amplified cDNA products encoding the respective variable region into the DNA fragment 3′ to the promoter sequence and 5′ to the corresponding constant domain sequence, thereby producing a pair of expression constructs for the heavy chain and the light chain of an antibody.

Embodiment 95. The method of any one of embodiments 39 to 94, wherein providing the biological cell within the microwell or sequestration pen is performed before providing the capture object within the microwell or sequestration pen.

Embodiment 96. The method of any one of embodiments 39 to 94, wherein providing the capture object within the chamber is performed before providing the biological cell within the chamber.

Embodiment 97. The method of any one of embodiments 39 to 96, further comprising providing each of one or more capture objects to each of a corresponding one of one or more chambers within the microfluidic device.

Embodiment 98. The method of one of embodiments 39 to 97, further comprising providing each of one or more biological cells to each of a corresponding one or more chambers of the microfluidic device.

Embodiment 99. The method of embodiment 98, wherein each one of the one or more biological cells are provided in a different one of the one or more chambers.

Embodiment 100. The method of any one of embodiments 98 to 99, wherein the one or more biological cells are provided within isolation regions of the one or more chambers of the microfluidic device, when the chambers comprise sequestration pens.

Embodiment 101. The method of any one of embodiments 98 to 100, wherein at least one of the one or more biological cells is provided within a chamber having one of the one or more capture objects provided therein.

Embodiment 102. The method of any one of embodiments 39 to 101, wherein the one or more biological cells is a plurality of biological cells from a clonal population.

Embodiment 103. The method of any one of embodiments 39 to 102, wherein providing the one or more biological cells is performed before providing the one or more capture objects.

Embodiment 104. The method of any one of embodiments 39 to 103, wherein the biological cell is an immune cell.

Embodiment 105. The method of any one of embodiments 39 to 103, wherein the biological cell is a cancer cell.

Embodiment 106. The method of any one of embodiments 39 to 103, wherein the biological cell is a stem cell or progenitor cell.

Embodiment 107. The method of any one of embodiments 39 to 103, wherein the biological cell is an embryo.

Embodiment 108. The method of any one of embodiments 39 to 107, wherein the biological cell is a single biological cell.

Embodiment 109. The method of any one of embodiments 39 to 108, wherein providing the biological cell further comprises marking the biological cell.

Embodiment 110. The method of any one of embodiments 39 to 109, wherein the microfluidic device further comprises a flow region for containing a flow of a first fluidic medium, and a microfluidic channel comprising at least a portion of the flow region.

Embodiment 111. The method of any one of embodiments 39 to 109, wherein the microfluidic device further comprises a flow region for containing a flow of a first fluidic medium; and a sequestration pen comprising an isolation region for containing a second fluidic medium, the isolation region having a single opening, wherein the isolation region of the sequestration pen is an unswept region of the microfluidic device; and a connection region fluidically connecting the isolation region to the flow region.

Embodiment 112. The method of any one of embodiments 39 to 111, wherein each of the one or more chambers of the microfluidic device has at least one inner surface coated with a coating material that provides a layer of organic and/or hydrophilic molecules.

Embodiment 113. The method of embodiment 110 or 111, wherein the flow region or channel of the microfluidic device has at least one inner surface coated with coating materials.

Embodiment 114. The method of embodiment 112 or 113, wherein the at least one coated surface comprises a hydrophilic or a negatively charged coated surface.

Embodiment 115. The method of any one of embodiments 39 to 114, wherein the enclosure of the microfluidic device further comprises a dielectrophoretic (DEP) configuration.

Embodiment 116. The method of embodiment 115, wherein providing the biological cell and/or providing the capture object is performed by applying a dielectrophoretic (DEP) force on or proximal to the biological cell and/or the capture object.

Embodiment 117. A method of preparing a construct for expression of the protein of interest, comprising: providing a barcoded cDNA sequence produced by the method of any one of embodiments 39 to 82, wherein the barcoded cDNA sequence comprises a nucleic acid encoding a protein of interest linked to the reverse complement of the barcode sequence of the first oligonucleotide; amplifying at least a portion of the barcoded cDNA sequence using a barcode-specific primer and a primer specific to the nucleic acid encoding the protein of interest, thereby producing an amplified cDNA product; providing a DNA fragment for transcriptionally active PCR (TAP), the DNA fragment comprising: a promoter sequence, a nucleic acid sequence complementary to a 5′ end of the nucleic acid encoding the protein of interest (e.g., 5′ end of the amplified cDNA product), a nucleic acid sequence complementary to a 3′ end of the nucleic acid encoding the protein of interest (e.g., a 3′ end of the amplified cDNA product), and a terminator sequence; and incorporating the amplified cDNA product into the DNA fragment for TAP, thereby producing a construct for expression of the protein of interest.

Embodiment 118. A method of preparing a construct for expression of an antibody, comprising: providing a barcoded cDNA sequence produced by the method of any one of embodiments 39 to 117, wherein the barcoded cDNA sequence comprises a nucleic acid encoding a heavy chain or a light chain of an antibody, or a fragment thereof, linked to the reverse complement of the barcode sequence of the first oligonucleotide; amplifying at least a portion of the barcoded cDNA sequence using a barcode-specific primer and a primer specific to the nucleic acid encoding the heavy chain or the light chain of the antibody, thereby producing an amplified cDNA product; providing a DNA fragment for transcriptionally active PCR (TAP), the DNA fragment comprising: a promoter sequence, a nucleic acid sequence complementary to a 5′ end of the nucleic acid encoding the heavy chain or light chain sequence (e.g., 5′ end of the amplified cDNA product), a nucleic acid sequence complementary to a 3′ end of the nucleic acid encoding the heavy chain or light chain sequence (e.g., a 3′ end of the amplified cDNA product), a heavy or light chain constant domain sequence, and a terminator sequence; incorporating the amplified cDNA product into the DNA fragment for TAP, thereby producing a construct for expression of the heavy chain or light chain of the antibody comprising a variable domain and a constant domain.

Embodiment 119. The method of embodiment 118, wherein the barcoded cDNA sequence comprises a nucleic acid encoding a heavy chain variable domain or a light chain variable domain of an antibody linked to a barcode sequence at a 5′ end.

Embodiment 120. The method of any one of embodiments 118 to 119, wherein the amplified cDNA product comprises a heavy chain variable domain or light chain variable domain sequence.

Embodiment 121. The method of any one of embodiments 118 to 120, wherein the DNA fragment for TAP comprises an antibody sequence encoding a heavy or light chain constant domain sequence 3′ to a respective variable region.

Embodiment 122. The method of any one of embodiments 118 to 121, wherein incorporating the amplified cDNA product into the DNA fragment for TAP comprises incorporating the amplified cDNA product encoding the variable region into the DNA fragment 3′ to the promoter sequence and 5′ to the sequence encoding the heavy or light chain constant domain sequence.

Embodiment 123. The method of any one of embodiments 118 to 122, wherein the constant region sequence in the DNA fragment for TAP is a heavy chain constant region sequence.

Embodiment 124. The method of embodiment 123, wherein the heavy chain constant region sequence comprises one, two, or three tandem immunoglobulin domains.

Embodiment 125. The method of any one of embodiments 118 to 124, wherein the constant region sequence in the DNA fragment for TAP is a light chain constant region sequence.

Embodiment 126. The method of any one of embodiments 118 to 125, wherein the promoter sequence comprises a cytomegalovirus (CMV) promoter sequence.

Embodiment 127. The method of any one of embodiments 118 to 126, wherein the promoter sequence provides constitutive gene expression.

Embodiment 128. The method of any one of embodiments 118 to 127, wherein the DNA fragment for TAP further comprises a sequence encoding fluorescent reporter protein.

Embodiment 129. The method of embodiment 128, wherein the DNA fragment for TAP further comprises a sequence encoding a self-cleaving peptide 5′ to the sequence encoding fluorescent reporter protein.

Embodiment 130. The method of embodiment 129, wherein the self-cleaving peptide is T2A, P2A, E2A, or F2A.

Embodiment 131. The method of embodiment 129, wherein the self-cleaving peptide is T2A.

Embodiment 132. The method of any one of embodiments 118 to 131, wherein amplifying the barcoded cDNA sequence occurs by performing polymerase chain reaction (PCR) selective for barcoded cDNA sequences using the barcode-specific primer.

Embodiment 133. The method of any one of embodiments 118 to 132, wherein incorporating the amplified barcoded cDNA sequence into the DNA fragment for TAP occurs by using overlap extension PCR.

Embodiment 134. The method of any one of embodiments 118 to 133, further comprising amplifying the expression construct.

Embodiment 135. A capture object comprising a label, a plurality of first and second oligonucleotides wherein each first oligonucleotide of the plurality comprises a barcode sequence, and a sequence comprising at least three consecutive guanine nucleotides at a 3′ end of each first oligonucleotide and wherein each second oligonucleotide of the plurality comprises a capture sequence.

Embodiment 136. The capture object of embodiment 135, wherein the first oligonucleotide comprises a first priming sequence that corresponds to a first primer sequence and/or wherein the second oligonucleotide comprises a second priming sequence that corresponds to a second primer sequence.

Embodiment 137. The capture object of embodiment 136, wherein the first and second primer sequences are the same.

Embodiment 138. The capture object of any one of embodiments 135 to 137, wherein the barcode sequence of the first oligonucleotide corresponds to the label of the capture object.

Embodiment 139. The capture object of embodiment 138, wherein the label is an integral color of the capture object.

Embodiment 140. The capture object of any one of embodiments 135 to 139, wherein the barcode sequence of the first oligonucleotide is the label of the capture object.

Embodiment 141. The capture object of any one of embodiments 135 to 140, wherein the label of the capture object comprises one or more fluorophores.

Embodiment 142. The capture object of embodiment 141, wherein the label comprises a single fluorophore.

Embodiment 143. The capture object of embodiment 141, wherein the label comprises multiple fluorophores.

Embodiment 144. The capture object of any one of embodiments 135 to 143, wherein the first oligonucleotide comprises one or more uridine nucleotides 5′ to the barcode sequence and, if present, the first priming sequence.

Embodiment 145. The capture object of any one of embodiments 135 to 144, wherein the first oligonucleotide comprises three uridine nucleotides 5′ to the barcode sequence and, if present, the first priming sequence.

Embodiment 146. The capture object of embodiment 144 or 145, wherein the one or more uridine nucleotides are adjacent to or comprise the 5′-most nucleotide(s) of the first oligonucleotide.

Embodiment 147. The capture object of any one of embodiments 135 to 146, wherein the capture sequence of the second oligonucleotide of the plurality of capture objects comprises an oligo-dT sequence (e.g., a (T)xVN sequence or a (T)xVI sequence, wherein X is greater than 10, 15, 20, 25, or 30).

Embodiment 148. The capture object of any one of embodiments 135 to 147, wherein the ratio of the second oligonucleotide to the first oligonucleotide on the capture object ranges from 1:10 to 10:1.

Embodiment 149. The capture object of any one of embodiments 135 to 148, wherein the ratio of the second oligonucleotide to the first oligonucleotide on the capture object is about 1:1 (e.g., 95:100 to 100:95).

Embodiment 150. The capture object of any one of embodiments 135 to 149, wherein the first oligonucleotide comprises, consists of, or consists essentially of RNA.

Embodiment 151. The capture object of any one of embodiments 135 to 150, wherein the first oligonucleotide comprises at least one modified base.

Embodiment 152. The capture object of embodiment 151, where the at least one modified base independently comprises a 2′-O-methyl base, O-methoxy-ethyl (MOE) base, or a locked nucleic acid base.

Embodiment 153. The capture object of any one of embodiments 135 to 152, wherein the first oligonucleotide comprises at least one phosphorothioate bond.

Embodiment 154. The capture object of any one of embodiments 135 to 153, wherein the first oligonucleotide is linked to the capture object.

Embodiment 155. The capture object of any one of embodiments 135 to 154, wherein the first oligonucleotide is covalently bound to the capture object.

Embodiment 156. The capture object of embodiment 154, wherein the first oligonucleotide is linked to the capture object by streptavidin-biotin binding.

Embodiment 157. The capture object of any one of embodiments 135 to 156, wherein the second oligonucleotide is linked to the capture object.

Embodiment 158. The capture object of any one of embodiments 135 to 157, wherein the second oligonucleotide is covalently bound to the capture object.

Embodiment 159. The capture object of embodiment 157, wherein the second oligonucleotide is linked to the capture object by streptavidin-biotin binding.

Embodiment 160. The capture object of any one of embodiments 135 to 159, wherein each of the one or more barcoded cDNA sequences is associated with the capture object (e.g. a capture object associated with one or more barcoded cDNA sequence can be a produced obtained by the method of any one of embodiments 39 to 116).

Embodiment 161. A plurality of capture objects, wherein each capture object of the plurality is a capture object according to any one of embodiment 135 to 160, wherein the barcode sequence of the first oligonucleotide of each capture object of the plurality is different from the barcode sequence of the first oligonucleotide of a capture object of the plurality having a different label.

Embodiment 162. The plurality of capture objects of embodiment 161, wherein the plurality comprises at least 4 types of capture objects.

Embodiment 163. The plurality of capture objects of embodiment 161, wherein the plurality comprises at least 8 types of capture objects.

Embodiment 164. The plurality of capture objects of embodiment 161, wherein the plurality comprises at least 12 types of capture objects.

Embodiment 165. A kit comprising a plurality of capture objects according to any one of embodiments 161 to 164.

Embodiment 166. A kit comprising (i) a microfluidic device having a plurality of chambers, and (ii) a plurality of capture objects, each having a plurality of first and second oligonucleotides, according to any one of embodiments 161 to 164.

Embodiment167. The kit according to embodiment 165 or 166, wherein the plurality of capture objects includes capture objects having at least 4 different barcodes (e.g., at least 4, 5, 6, 7, 8, 9, 10, 11, 12, 14, 16, 18, 20, 25, 30, 40, 50, 60, 70, 80, 90, 100, 150, 200, 250, 500, 1000, or more different barcodes).

Embodiment 168. The kit according to any one of embodiments 165 to 166, further comprising reverse transcription enzyme, USER enzyme, a lytic agent (e.g., a lysis buffer), one or more surface conditioning agents (e.g., for conditioning the inner surfaces of the chip), or any combination thereof.

Embodiment 169. The kit according to any one of embodiments 165 to 168, wherein the plurality of capture objects are in a solution comprising an RNAse inhibitor.

Embodiment 170. The kit according to embodiment 169, wherein the RNAse inhibitor is a chemical base RNAse inhibitor.

Embodiment 171. The kit according to any one of embodiments 165 to 170, wherein the plurality of capture objects are stored at a temperature of about 4° C.

Embodiment 172. A method of introducing a micro-object into a chamber of a microfluidic device, comprising: introducing one or more micro-objects into a flow region of a microfluidic device; determining a condition of viability of the one or more micro-objects; selecting at least one micro-object having viability from the one or more micro-objects; and introducing the at least one micro-object into a chamber of the microfluidic device.

Embodiment 173. The method of embodiment 172, wherein introducing the at least one micro-object into the chamber comprises using DEP force.

Embodiment 174. The method of embodiment 172 or 173, wherein determining the condition of viability comprises employing a machine-learning algorithm to assign a probability of viability to each of the one or more micro-objects.

Embodiment 175. The method of embodiment 174, wherein the machine-learning algorithm comprises a trained machine-learning algorithm, wherein training the machine-learning algorithm comprises imaging micro-objects comprising a label demarking a condition of viability.

Embodiment 176. The method of embodiment 175, wherein the micro-objects comprising the label demarking viability are a same type of cells as the one or more micro-objects.

Embodiment 177. The method of embodiment 175 or 176, wherein the label comprises a live/dead stain comprising calcein, zombie violet stain, annexin, acridine orange, propidium iodide, or any combination thereof.

Embodiment 178. The method of any one of embodiments 175 to 177, wherein the training further comprises imaging the micro-objects comprising the label under brightfield conditions.

Embodiment 179. The method of any one of embodiments 172 to 178, wherein the one or more micro-objects comprises a plurality of micro-objects and the at least one micro-object introduced to the chamber comprises a sub-set of the plurality of micro-objects.

Embodiment 180. The method of any one of embodiments 172- 179, wherein the chamber comprises a sequestration pen.

Embodiment 181. A method for assembling a sequence from sequence fragments for a nucleic acid obtained from a biological micro-object or a clonal population thereof, comprising:obtaining a plurality of sequence fragments, wherein a subset of the plurality of sequence fragments is derived from the nucleic acid obtained from the biological micro-object or a clonal population thereof;
aligning the subset of sequence fragments with a reference sequence;
determining, from alignments between each sequence fragment of the subset of sequence fragments and the reference sequence, a matching frequency between each base of each sequence fragment of the subset of sequence fragments and each corresponding base of the reference sequence; and
constructing a sequence by selecting a base having the highest matching frequency at each position of the constructed sequence.

Embodiment 182. The method of embodiment 181, further comprising determining, from an alignment between each sequence fragment of the subset of sequence fragments and the reference sequence, a mismatching frequency between each base of each sequence fragment of the subset of sequence fragments and each corresponding base of the reference sequence.

Embodiment 183. The method of embodiment 181 or 182, further comprising determining an alternation and a corresponding alternation frequency from alignments between each sequence fragment of the subset of sequence fragments and the reference sequence; wherein the alternation comprises an insertion and/or a deletion.

Embodiment 184. The method of embodiment 183, wherein constructing the sequence comprises modifying the constructed sequence based on the alternation.

Embodiment 185. The method of embodiment 184, wherein the alternation comprises an insertion; and wherein the constructed sequence is modified with the insertion provided that the alternation frequency for the insertion is at least half a frequency value of a base prior to (e.g., immediately 5′ to) the insertion and a base following (e.g., immediately 3′ to) the insertion.

Embodiment 186. The method of embodiment 184 or 185, wherein the alternation comprises a deletion; and wherein the constructed sequence is modified with the deletion provided that the alternation frequency of the deletion is greater than the frequency of any base removed by the deletion.

Embodiment 187. The method of any one of embodiments 181 to 186, wherein the reference sequence comprises a plurality of reference sequences.

Embodiment 188. The method of any one of embodiments 181 to 187, wherein the subset of sequence fragments is derived from a heavy chain of an antibody; and further wherein the subset of heavy chain sequence fragments comprises a plurality of heavy V allele sequence fragments, a plurality of heavy D allele sequence fragments, and a plurality of heavy J allele sequence fragments.

Embodiment 189. The method of embodiment 188, wherein aligning the plurality of heavy chain sequence fragments with a reference sequence further comprises:
aligning the subset of sequence fragments with each of a set of heavy V reference sequences, thereby identifying one or more observed heavy V allele sequences, and
aligning the subset of sequence fragments with each of a set of heavy J reference sequence, thereby identifying one or more observed heavy J allele sequences.

Embodiment 190. The method of embodiment 189, wherein the set of heavy V reference sequence comprises more than one distinct heavy V reference sequence.

Embodiment 191.The method of embodiment 189 or 190, wherein the set of heavy J reference sequence comprises more than one distinct heavy J reference sequence.

Embodiment 192. The method of any one of embodiments 189 to 191, further comprising creating a set of heavy CDR3 reference sequences; wherein the set of heavy CDR3 reference sequences comprises at least one extended heavy CDR3 sequence region; and further wherein each of the at least one extended heavy CDR3 sequence region comprises a combination of:
a heavy V allele end sequence (e.g., 3′ end sequence) derived from one of the one or more observed heavy V allele sequences;
one of the plurality of heavy D allele sequence fragments; and
a heavy J allele starting sequence (e.g., 5′ start sequence) derived from one of the one or more observed heavy J allele sequences; wherein the combined sequences are provided in an order of V allele, D allele, J allele in each sequence, and optionally, wherein the set of heavy CDR3 reference sequences comprises a plurality (e.g., 2, 3, 4, 5 or more, 10 or more, 15 or more, 20 or more, or all possible) of combinations of the foregoing heavy V allele end sequences, plurality of heavy D allele sequence fragments, and heavy J allele start sequences.

Embodiment 193. The method of embodiment 192, wherein the heavy V allele ending sequence comprises at least the last 10, (or 15, 25, 30, 35, 40, 45, 50, 55, 60 or more) bases of one of the one or more plurality of observed heavy V allele sequences; and wherein the heavy J allele starting sequence comprises at least the first 10, (or 15, 25, 30, 35, 40, 45, 50, 55, 60 or more) bases of one of the one or more of observed heavy J allele sequences.

Embodiment 194. The method of any one of embodiments 192 to 193, wherein aligning the plurality of sequence fragments with a reference sequence comprises: aligning the plurality of sequence fragments with each sequence of the set of the heavy CDR3 reference sequences ; and constructing a sequence comprises assembling a set of observed extended heavy CDR3 sequences.

Embodiment 195. The method of embodiment 194, further comprising assembling a possible full-length variable heavy chain sequence, comprising:
aligning each of the one or more observed heavy V allele sequences with each sequence of the set of observed extended heavy CDR3 sequences, and thereby identifying one of the one or more observed heavy V allele sequences comprising a 3′ terminus sequence that most strongly overlaps with a 5′ end sequence of one of the set of observed extended heavy CDR3 sequences;
aligning each of one or more observed heavy J allele sequences with each of the set of observed extended heavy CDR3 sequences, and thereby identifying one of the one or more observed heavy J allele sequences comprising a 5′ terminus sequence that most strongly overlaps with a 3′ end sequence of one of the set of observed extended heavy CDR3 sequences; and
in accordance with the most strongly overlapping sequences, constructing the possible full-length variable heavy chain sequence from the identified one of the one or more observed heavy V allele sequences, the identified one of the one or more observed heavy J allele sequence, and the one of the set of observed extended heavy CDR3 sequences used for such identifying.

Embodiment 196. The method of any one of embodiments 181 to 195, wherein the subset of sequence fragments is derived from a light chain of an antibody and wherein the subset of sequence fragments comprises a plurality of light V allele sequence fragments, and a plurality of light J allele sequence fragments.

Embodiment 197. The method of embodiment 196, wherein aligning the subset of light chain sequence fragments with a reference sequence further comprises:
aligning the subset of sequence fragments with each of a set of light V reference sequences, thereby identifying one or more observed light V allele sequences, and
aligning the plurality of sequence fragments with each of a set of light J reference sequences, thereby identifying one or more observed light J allele sequences.

Embodiment 198. The method of embodiment 197, wherein the set of light V reference sequences comprises more than one distinct light V reference sequence.

Embodiment 199. The method of embodiment 197 or 198, wherein the set of light J reference sequences comprises more than one distinct light J reference sequence.

Embodiment 200. The method of any one of embodiments 196 to 199, further comprising creating a set of light CDR3 reference sequences; wherein the set of light CDR3 reference sequences comprises at least one extended light CDR3 sequence region; and further wherein each of the at least one extended light CDR3 sequence region comprises a combination of:
a light V allele end sequence (e.g., 3′ end sequence) derived from one of the one or more of observed light V allele sequences; and
a light J allele start sequence (e.g., 5′ start sequence) derived from one of the one or more of observed light J allele sequences; wherein the combined sequences are provided in an order of V allele, J allele in each sequence, and optionally wherein the set of light CDR3 reference sequences comprises a plurality of (e.g., 2, 3, 4, 5 or more, 10 or more, 15 or more, 20 or more, or all possible) combinations of the foregoing light V allele end sequences and light J allele start sequences.

Embodiment 201. The method of embodiment 200, wherein the light V allele end sequence comprises at least the last 10 (or 15, 25, 30, 35, 40, 45, 50, 55, 60 or more)bases of one of the plurality of observed light V allele sequences; and wherein the light J allele start sequence comprises at least the first 10 (or 15, 25, 30, 35, 40, 45, 50, 55, 60 or more) bases of one of the one or more of observed light J allele sequences.

Embodiment 202. The method of any one of embodiments 200 to 201, wherein aligning the plurality of sequence fragments with a reference sequence comprises: aligning the plurality of sequence fragments with each sequence of the set of light CDR3 reference sequences ; and constructing a sequence comprises assembling a set of observed extended light CDR3 sequences.

Embodiment 203. The method of embodiment 202, further comprising: assembling a possible full-length variable light chain sequence, comprising:
aligning each of the one or more observed light V allele sequences with each sequence of the set of observed extended light CDR3 sequences, and thereby identifying one of the one or more observed heavy V allele sequences comprising a 3′ terminus sequence that most strongly overlaps with a 5′ end sequence of one of the set of observed extended light CDR3 sequences;
aligning each of the one or more observed light J allele sequences with each sequence of the set of observed extended light CDR3 sequences, and thereby identifying one of the one or more observed light J allele sequences comprising a 5′ terminus sequence that most strongly overlaps with a 3′ end sequence of one of the set of observed extended light CDR3 sequences; and
in accordance with the most strongly overlapping sequences, constructing the possible full-length variable light chain sequence from the identified one of the one or more observed light V allele sequences, the identified one of the one or more observed light J allele sequences, and the one of the set of observed extended light CDR3 sequences used for such identifying

Embodiment 204. The method of embodiment 203, further comprising constructing a possible heavy and light chain sequences for the nucleic acid obtained from the biological micro-object or the clonal population thereof by obtaining a combined reference set, comprising the possible full-length variable heavy chain sequences and the possible full-length variable light chain sequences.

### XV. Description of Sequences

Table 8 provides a listing of certain sequences referenced herein

| Description | Sequence | SEQ ID No. |
|---|---|---|
| Exemplary barcode | TGGTAGGCTG | 1 |
| Exemplary barcode | GTTAGCTGCT | 2 |
| Exemplary barcode | TACATAAAGA | 3 |
| Exemplary barcode | AGCCCTATCA | 4 |
| Exemplary barcode | ACCTACCGCC | 5 |
| Exemplary barcode | TCTCCAAGAC | 6 |
| Exemplary barcode | GTATACATTA | 7 |
| Exemplary barcode | AGACTCGATT | 8 |
| Exemplary barcode | CCAGGATTAA | 9 |
| Exemplary barcode | CTCCTTCAAG | 10 |
| Exemplary barcode | ACTACTTCTG | 11 |
| Exemplary barcode | GCCTTGTTGT | 12 |
| Exemplary demultiplexing forward primer | CTTCCGATCTTGGTAGGCTG | 13 |
| Exemplary demultiplexing forward primer | CTTCCGATCTGTTAGCTGCT | 14 |
| Exemplary demultiplexing forward primer | CTTCCGATCTACATAAAGA | 15 |
| Exemplary demultiplexing forward primer | CTTCCGATCTAGCCCTATCA | 16 |
| Exemplary demultiplexing forward primer | CTTCCGATCTACCTACCGCC | 17 |
| Exemplary demultiplexing forward primer | CTTCCGATCTTCTCCAAGAC | 18 |
| Exemplary demultiplexing forward primer | CTTCCGATCTGTATACATTA | 19 |
| Exemplary demultiplexing forward primer | CTTCCGATCTAGACTCGATT | 20 |
| Exemplary demultiplexing forward primer | CTTCCGATCTCCAGGATTAA | 21 |
| Exemplary demultiplexing forward primer | CTTCCGATCTCTCCTTCAAG | 22 |
| Exemplary demultiplexing forward primer | CTTCCGATCTACTACTTCTG | 23 |
| Exemplary demultiplexing forward primer | CTTCCGATCTGCCTTGTTGT | 24 |
| Exemplary barcode-specific primer | CTCACACGACGCTCTTCCGATCTTGGTAGGCTG | 25 |
| Exemplary barcode-specific primer | CTCACACGACGCTCTTCCGATCTGTTAGCTGCT | 26 |
| Exemplary barcode-specific primer | CTCACACGACGCTCTTCCGATCTTACATAAAGA | 27 |
| Exemplary barcode-specific primer | CTCACACGACGCTCTTCCGATCTAGCCCTATCA | 28 |
| Exemplary barcode-specific primer | CTCACACGACGCTCTTCCGATCTACCTACCGCC | 29 |
| Exemplary barcode-specific primer | CTCACACGACGCTCTTCCGATCTTCTCCAAGAC | 30 |
| Exemplary barcode-specific primer | CTCACACGACGCTCTTCCGATCTGTATACATTA | 31 |
| Exemplary barcode-specific primer | CTCACACGACGCTCTTCCGATCTAGACTCGATT | 32 |
| Exemplary barcode-specific primer | CTCACACGACGCTCTTCCGATCTCCAGGATTAA | 33 |
| Exemplary barcode-specific primer | CTCACACGACGCTCTTCCGATCTCTCCTTCAAG | 34 |
| Exemplary barcode-specific primer | CTCACACGACGCTCTTCCGATCTACTACTTCTG | 35 |
| Exemplary barcode-specific primer | CTCACACGACGCTCTTCCGATCTGCCTTGTTGT | 36 |
| Exemplary first oligonucleotide | /52-Bio/TATATAUUUGTGGTATCAACGCAGAGTACACGAC GCTCTTCCGATCTTGGTAGGCTGmG*mG*mG* | 37 |
| Exemplary first oligonucleotide | /52-Bio/TATATAUUUGTGGTATCAACGCAGAGTACACGAC GCTCTTCCGATCTGTTAGCTGCTmG*mG*mG* | 38 |
| Exemplary first oligonucleotide | /52-Bio/TATATAUUUGTGGTATCAACGCAGAGTACACGAC GCTCTTCCGATCTTACATAAAGAmG*mG*mG* | 39 |
| Exemplary first oligonucleotide | /52-Bio/TATATAUUUGTGGTATCAACGCAGAGTACACGAC GCTCTTCCGATCTAGCCCTATCAmG*mG*mG* | 40 |
| Exemplary first oligonucleotide | /52-Bio/TATATAUUUGTGGTATCAACGCAGAGTACACGAC GCTCTTCCGATCTACCTACCGCCmG*mG*mG* | 41 |
| Exemplary first oligonucleotide | /52-Bio/TATATAUUUGTGGTATCAACGCAGAGTACACGAC GCTCTTCCGATCTTCTCCAAGACmG*mG*mG* | 42 |
| Exemplary first oligonucleotide | /52-Bio/TATATAUUUGTGGTATCAACGCAGAGTACACGAC GCTCTTCCGATCTGTATACATTAmG*mG*mG* | 43 |
| Exemplary first oligonucleotide | /52-Bio/TATATAUUUGTGGTATCAACGCAGAGTACACGAC GCTCTTCCGATCTAGACTCGATTmG*mG*mG* | 44 |
| Exemplary first oligonucleotide | /52-Bio/TATATAUUUGTGGTATCAACGCAGAGTACACGAC GCTCTTCCGATCTCCAGGATTAAmG*mG*mG* | 45 |
| Exemplary first oligonucleotide | /52-Bio/TATATAUUUGTGGTATCAACGCAGAGTACACGAC GCTCTTCCGATCTCTCCTTCAAGmG*mG*mG* | 46 |
| Exemplary first oligonucleotide | /52-Bio/TATATAUUUGTGGTATCAACGCAGAGTACACGAC GCTCTTCCGATCTACTACTTCTGmG*mG*mG* | 47 |
| Exemplary first oligonucleotide | /52-Bio/TATATAUUUGTGGTATCAACGCAGAGTACACGAC GCTCTTCCGATCTGCCTTGTTGTmG*mG*mG* | 48 |
| Exemplary second oligonucleotide including a capture sequence | /5Biosg/AAGCAGTGGTATCAACGCAGAGTACTTTTT TTTTTTTTTTTTTTTTTTTTTTTTTVI | 49 |
| Exemplary priming sequence | AAGCAGTGGTATCAACGCAGAGTAC | 50 |
| Exemplary priming sequence | ACACTCTTTCCCTACACGACGCTCTTCCGATC | 51 |
| Exemplary priming sequence | AATGATACGGCGACCACCGAGATCTACACTCTTTCCCT ACACGA | 52 |
| Exemplary priming sequence | CAAGCAGAAGACGGCATACGAGAT | 53 |
| Exemplary heavy chain constant region reverse primer | ACAGTCACTGAGCTGCT | 54 |
| Exemplary light chain constant region reverse primer | GACTGAGGCACCTCCAGATG | 55 |
| Not1 restriction site sequence | GCGGCCGC | 56 |
| Exemplary barcode-specific primer | cttccgatct tggtaggctg | 57 |
| Exemplary cDNA sequence | acacgacgct cttccgatct tggtaggctg | 58 |
| Exemplary cDNA sequence | cagcctacca agatcggaag agcgtcgtgt | 59 |
| Exemplary TAP adapter primer | agagtacacg acgctcttcc gatcttggta ggctg | 60 |
| Exemplary cDNA sequence | ctcttccgat cttggtaggc tg | 61 |
| Exemplary cDNA sequence | cagcctacca agatcggaag ag | 62 |
| Exemplary TAP backbone sequence | tatatatttg tggtatcaac gcagagtaca cgacgctctt ccgatct | 63 |
| Exemplary cDNA sequence | tctcatgtgc tgcgagaagg ctagaaccat ccgac | 64 |

^{∗}= PS linkage; m = 2′-O-methyl

What is claimed:

1-18. (canceled)

19. A method of providing one or more barcoded cDNA sequences from a biological cell, comprising:
providing the biological cell within a chamber;
providing a capture object in the chamber, the capture object comprising a label, a plurality of first oligonucleotides, and a plurality of second oligonucleotides,
wherein each first oligonucleotide of the plurality comprises a barcode sequence, and a sequence comprising at least three consecutive guanine nucleotides at a 3′ end of each first oligonucleotide,
wherein each second oligonucleotide of the plurality comprises a capture sequence, lysing the biological cell and allowing RNA released therefrom the lysed biological cell to be captured by the capture sequences of the plurality of second oligonucleotides, thereby forming captured RNA; and
reverse transcribing the captured RNA, thereby producing one or more barcoded cDNA sequences, each comprising an oligonucleotide sequence complementary to a corresponding one of the captured RNA and covalently linked to the reverse complement of the barcode sequence of the first oligonucleotide.

20. The method of claim 19, wherein the chamber comprises a microwell or a sequestration pen of a microfluidic device.

21. The method of claim 19, wherein a single capture object is provided in the chamber.

22. The method of claim 19, wherein the capture sequence binds to, and thereby, captures RNA and primes transcription from the captured RNA.

23. The method of claim 19, further comprising identifying the barcode sequence of the plurality of first oligonucleotides while the capture object is located within the chamber.

24. The method of claim 19, wherein the first oligonucleotide comprises one or more uridine nucleotides 5′ to the barcode sequence, wherein reverse transcribing the captured RNA is performed in the presence of an enzyme that cleaves a sequence containing one or more uridine nucleotides.

25. (canceled)

26. The method of claim 19, wherein the ratio of the second oligonucleotide to the first oligonucleotide on the capture object ranges from 1:10 to 10:1.

27-29. (canceled)

30. The method of claim 19, further comprising exporting the capture object from the chamber.

31. The method of claim 19, wherein providing one or more barcoded cDNA sequences comprises providing a plurality of barcoded cDNA sequences, each barcoded cDNA sequence of the plurality encoding a protein of interest, corresponding to any one of a plurality of different proteins, linked to a corresponding reverse complement barcode sequence; and the method further comprising:
selectively amplifying the plurality of barcoded cDNA sequences (or amplified cDNA sequences) using a barcode-specific forward primer and a reverse primer specific to the protein of interest to produce an amplified cDNA product (or further amplified cDNA product) encoding the protein of interest or a fragment thereof;
annealing a 5′ end of the amplified cDNA product (or further amplified cDNA product) to a 5′ corresponding end of a DNA fragment for transcriptionally-active PCR (TAP) to produce an annealed TAP product; and
amplifying the annealed TAP product via overlap extension PCR using a TAP adapter primer to produce a construct for expression of the protein of interest.

32. (canceled)

33. A method of preparing a construct for expression of an antibody, comprising:
providing a barcoded cDNA sequence produced by the method of claim 19, wherein the barcoded cDNA sequence comprises a nucleic acid encoding a heavy chain or a light chain of an antibody, or a fragment thereof, linked to the reverse complement of the barcode sequence of the first oligonucleotide;
amplifying at least a portion of the barcoded cDNA sequence using a barcode-specific primer and a primer specific to the nucleic acid encoding the heavy chain or the light chain of the antibody, thereby producing an amplified cDNA product;
providing a DNA fragment for transcriptionally active PCR (TAP), the DNA fragment comprising:
a promoter sequence,
a nucleic acid sequence complementary to a 5′ end of the nucleic acid encoding the heavy chain or light chain sequence,
a nucleic acid sequence complementary to a 3′ end of the nucleic acid encoding the heavy chain or light chain sequence,
a heavy or light chain constant domain sequence, and
a terminator sequence;
incorporating the amplified cDNA product into the DNA fragment for TAP, thereby producing a construct for expression of the heavy chain or light chain of the antibody
comprising a variable domain and a constant domain.

34. The method of claim 33, wherein the DNA fragment for TAP comprises an antibody sequence encoding a heavy or light chain constant domain sequence 3′ to a respective variable region.

35. The method of claim 33 wherein incorporating the amplified cDNA product into the DNA fragment for TAP comprises incorporating the amplified cDNA product encoding the variable region into the DNA fragment 3′ to the promoter sequence and 5′ to the sequence encoding the heavy or light chain constant domain sequence.

36. The method of claim 33, wherein incorporating the amplified barcoded cDNA sequence into the DNA fragment for TAP occurs by using overlap extension PCR.

37. A capture object comprising a label, a plurality of first and second oligonucleotides wherein each first oligonucleotide of the plurality comprises a barcode sequence, and a sequence comprising at least three consecutive guanine nucleotides at a 3′ end of each first oligonucleotide and wherein each second oligonucleotide of the plurality comprises a capture sequence; wherein the first oligonucleotide comprises a first priming sequence that corresponds to a first primer sequence and/or wherein the second oligonucleotide comprises a second priming sequence that corresponds to a second primer sequence; wherein the barcode sequence of the first oligonucleotide corresponds to the label of the capture object.

38. The capture object of claim 37, wherein the first and second primer sequences are the same.

39. The capture object of claim 37, wherein the label is an integral color of the capture object.

40. The capture object of claim 37, wherein the ratio of the second oligonucleotide to the first oligonucleotide on the capture object ranges from 1:10 to 10:1.

41. The capture object of claim 37, wherein the ratio of the second oligonucleotide to the first oligonucleotide on the capture object is about 1:1.

42. The capture object of claim 37, wherein the first oligonucleotide is linked to the capture object.

43. The capture object of claim 37, wherein the second oligonucleotide is linked to the capture object.

44-52. (canceled)


SEQUENCE LISTING (64 sequences)

SEQ ID NO: 1 - DNA, 10 bp, synthetic construct
  misc_feature 1..10: note = Exemplary barcode
  source 1..10: mol_type = other DNA
tggtaggctg

SEQ ID NO: 2 - DNA, 10 bp, synthetic construct
  misc_feature 1..10: note = Exemplary barcode
  source 1..10: mol_type = other DNA
gttagctgct

SEQ ID NO: 3 - DNA, 10 bp, synthetic construct
  misc_feature 1..10: note = Exemplary barcode
  source 1..10: mol_type = other DNA
tacataaaga

SEQ ID NO: 4 - DNA, 10 bp, synthetic construct
  misc_feature 1..10: note = Exemplary barcode
  source 1..10: mol_type = other DNA
agccctatca

SEQ ID NO: 5 - DNA, 10 bp, synthetic construct
  misc_feature 1..10: note = Exemplary barcode
  source 1..10: mol_type = other DNA
acctaccgcc

SEQ ID NO: 6 - DNA, 10 bp, synthetic construct
  misc_feature 1..10: note = Exemplary barcode
  source 1..10: mol_type = other DNA
tctccaagac

SEQ ID NO: 7 - DNA, 10 bp, synthetic construct
  misc_feature 1..10: note = Exemplary barcode
  source 1..10: mol_type = other DNA
gtatacatta

SEQ ID NO: 8 - DNA, 10 bp, synthetic construct
  misc_feature 1..10: note = Exemplary barcode
  source 1..10: mol_type = other DNA
agactcgatt

SEQ ID NO: 9 - DNA, 10 bp, synthetic construct
  misc_feature 1..10: note = Exemplary barcode
  source 1..10: mol_type = other DNA
ccaggattaa

SEQ ID NO: 10 - DNA, 10 bp, synthetic construct
  misc_feature 1..10: note = Exemplary barcode
  source 1..10: mol_type = other DNA
ctccttcaag

SEQ ID NO: 11 - DNA, 10 bp, synthetic construct
  misc_feature 1..10: note = Exemplary barcode
  source 1..10: mol_type = other DNA
actacttctg

SEQ ID NO: 12 - DNA, 10 bp, synthetic construct
  misc_feature 1..10: note = Exemplary barcode
  source 1..10: mol_type = other DNA
gccttgttgt

SEQ ID NO: 13 - DNA, 20 bp, synthetic construct
  misc_feature 1..20: note = Exemplary demultiplexing forward primer
  source 1..20: mol_type = other DNA
cttccgatcttggtaggctg

SEQ ID NO: 14 - DNA, 20 bp, synthetic construct
  misc_feature 1..20: note = Exemplary demultiplexing forward primer
  source 1..20: mol_type = other DNA
cttccgatctgttagctgct

SEQ ID NO: 15 - DNA, 20 bp, synthetic construct
  misc_feature 1..20: note = Exemplary demultiplexing forward primer
  source 1..20: mol_type = other DNA
cttccgatcttacataaaga

SEQ ID NO: 16 - DNA, 20 bp, synthetic construct
  misc_feature 1..20: note = Exemplary demultiplexing forward primer
  source 1..20: mol_type = other DNA
cttccgatctagccctatca

SEQ ID NO: 17 - DNA, 20 bp, synthetic construct
  misc_feature 1..20: note = Exemplary demultiplexing forward primer
  source 1..20: mol_type = other DNA
cttccgatctacctaccgcc

SEQ ID NO: 18 - DNA, 20 bp, synthetic construct
  misc_feature 1..20: note = Exemplary demultiplexing forward primer
  source 1..20: mol_type = other DNA
cttccgatcttctccaagac

SEQ ID NO: 19 - DNA, 20 bp, synthetic construct
  misc_feature 1..20: note = Exemplary demultiplexing forward primer
  source 1..20: mol_type = other DNA
cttccgatctgtatacatta

SEQ ID NO: 20 - DNA, 20 bp, synthetic construct
  misc_feature 1..20: note = Exemplary demultiplexing forward primer
  source 1..20: mol_type = other DNA
cttccgatctagactcgatt

SEQ ID NO: 21 - DNA, 20 bp, synthetic construct
  misc_feature 1..20: note = Exemplary demultiplexing forward primer
  source 1..20: mol_type = other DNA
cttccgatctccaggattaa

SEQ ID NO: 22 - DNA, 20 bp, synthetic construct
  misc_feature 1..20: note = Exemplary demultiplexing forward primer
  source 1..20: mol_type = other DNA
cttccgatctctccttcaag

SEQ ID NO: 23 - DNA, 20 bp, synthetic construct
  misc_feature 1..20: note = Exemplary demultiplexing forward primer
  source 1..20: mol_type = other DNA
cttccgatctactacttctg

SEQ ID NO: 24 - DNA, 20 bp, synthetic construct
  misc_feature 1..20: note = Exemplary demultiplexing forward primer
  source 1..20: mol_type = other DNA
cttccgatctgccttgttgt

SEQ ID NO: 25 - DNA, 33 bp, synthetic construct
  misc_feature 1..33: note = Exemplary barcode-specific primer
  source 1..33: mol_type = other DNA
ctcacacgacgctcttccgatcttggtaggctg

SEQ ID NO: 26 - DNA, 33 bp, synthetic construct
  misc_feature 1..33: note = Exemplary barcode-specific primer
  source 1..33: mol_type = other DNA
ctcacacgacgctcttccgatctgttagctgct

SEQ ID NO: 27 - DNA, 33 bp, synthetic construct
  misc_feature 1..33: note = Exemplary barcode-specific primer
  source 1..33: mol_type = other DNA
ctcacacgacgctcttccgatcttacataaaga

SEQ ID NO: 28 - DNA, 33 bp, synthetic construct
  misc_feature 1..33: note = Exemplary barcode-specific primer
  source 1..33: mol_type = other DNA
ctcacacgacgctcttccgatctagccctatca

SEQ ID NO: 29 - DNA, 33 bp, synthetic construct
  misc_feature 1..33: note = Exemplary barcode-specific primer
  source 1..33: mol_type = other DNA
ctcacacgacgctcttccgatctacctaccgcc

SEQ ID NO: 30 - DNA, 33 bp, synthetic construct
  misc_feature 1..33: note = Exemplary barcode-specific primer
  source 1..33: mol_type = other DNA
ctcacacgacgctcttccgatcttctccaagac

SEQ ID NO: 31 - DNA, 33 bp, synthetic construct
  misc_feature 1..33: note = Exemplary barcode-specific primer
  source 1..33: mol_type = other DNA
ctcacacgacgctcttccgatctgtatacatta

SEQ ID NO: 32 - DNA, 33 bp, synthetic construct
  misc_feature 1..33: note = Exemplary barcode-specific primer
  source 1..33: mol_type = other DNA
ctcacacgacgctcttccgatctagactcgatt

SEQ ID NO: 33 - DNA, 33 bp, synthetic construct
  misc_feature 1..33: note = Exemplary barcode-specific primer
  source 1..33: mol_type = other DNA
ctcacacgacgctcttccgatctccaggattaa

SEQ ID NO: 34 - DNA, 33 bp, synthetic construct
  misc_feature 1..33: note = Exemplary barcode-specific primer
  source 1..33: mol_type = other DNA
ctcacacgacgctcttccgatctctccttcaag

SEQ ID NO: 35 - DNA, 33 bp, synthetic construct
  misc_feature 1..33: note = Exemplary barcode-specific primer
  source 1..33: mol_type = other DNA
ctcacacgacgctcttccgatctactacttctg

SEQ ID NO: 36 - DNA, 33 bp, synthetic construct
  misc_feature 1..33: note = Exemplary barcode-specific primer
  source 1..33: mol_type = other DNA
ctcacacgacgctcttccgatctgccttgttgt

SEQ ID NO: 37 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatcttggtaggctgggg

SEQ ID NO: 38 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctgttagctgctggg

SEQ ID NO: 39 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatcttacataaagaggg

SEQ ID NO: 40 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctagccctatcaggg

SEQ ID NO: 41 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctacctaccgccggg

SEQ ID NO: 42 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatcttctccaagacggg

SEQ ID NO: 43 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctgtatacattaggg

SEQ ID NO: 44 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctagactcgattggg

SEQ ID NO: 45 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctccaggattaaggg

SEQ ID NO: 46 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctctccttcaagggg

SEQ ID NO: 47 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctactacttctgggg

SEQ ID NO: 48 - DNA, 60 bp, synthetic construct
  misc_feature 1..60: note = Exemplary first oligonucleotide
  misc_feature 1: note = 5' Dual Biotin Tag
  misc_feature 7..9: note = n is u
  modified_base 58..60: mod_base = OTHER; note = 2'-O-Me
  modified_base 58..60: mod_base = OTHER; note = PS linkage
  source 1..60: mol_type = other DNA
tatatannngtggtatcaacgcagagtacacgacgctcttccgatctgccttgttgtggg

SEQ ID NO: 49 - DNA, 57 bp, synthetic construct
  misc_feature 1..57: note = Exemplary second oligonucleotide including a capture sequence
  misc_feature 1: note = 5' Biotin Tag
  misc_feature 57: note = n is inosine
  source 1..57: mol_type = other DNA
aagcagtggtatcaacgcagagtacttttttttttttttttttttttttttttttvn

SEQ ID NO: 50 - DNA, 25 bp, synthetic construct
  misc_feature 1..25: note = Exemplary priming sequence
  source 1..25: mol_type = other DNA
aagcagtggtatcaacgcagagtac

SEQ ID NO: 51 - DNA, 32 bp, synthetic construct
  misc_feature 1..32: note = Exemplary priming sequence
  source 1..32: mol_type = other DNA
acactctttccctacacgacgctcttccgatc

SEQ ID NO: 52 - DNA, 44 bp, synthetic construct
  misc_feature 1..44: note = Exemplary priming sequence
  source 1..44: mol_type = other DNA
aatgatacggcgaccaccgagatctacactctttccctacacga

SEQ ID NO: 53 - DNA, 24 bp, synthetic construct
  misc_feature 1..24: note = Exemplary priming sequence
  source 1..24: mol_type = other DNA
caagcagaagacggcatacgagat

SEQ ID NO: 54 - DNA, 17 bp, synthetic construct
  misc_feature 1..17: note = Exemplary heavy chain constant region reverse primer
  source 1..17: mol_type = other DNA
acagtcactgagctgct

SEQ ID NO: 55 - DNA, 20 bp, synthetic construct
  misc_feature 1..20: note = Exemplary light chain constant region reverse primer
  source 1..20: mol_type = other DNA
gactgaggcacctccagatg

SEQ ID NO: 56 - skipped

SEQ ID NO: 57 - DNA, 20 bp, synthetic construct
  misc_feature 1..20: note = Exemplary barcode specific primer
  source 1..20: mol_type = other DNA
cttccgatcttggtaggctg

SEQ ID NO: 58 - DNA, 30 bp, synthetic construct
  misc_feature 1..30: note = Exemplary cDNA sequence
  source 1..30: mol_type = other DNA
acacgacgctcttccgatcttggtaggctg

SEQ ID NO: 59 - DNA, 30 bp, synthetic construct
  misc_feature 1..30: note = Exemplary cDNA sequence
  source 1..30: mol_type = other DNA
cagcctaccaagatcggaagagcgtcgtgt

SEQ ID NO: 60 - DNA, 35 bp, synthetic construct
  misc_feature 1..35: note = Exemplary TAP adapter primer
  source 1..35: mol_type = other DNA
agagtacacgacgctcttccgatcttggtaggctg

SEQ ID NO: 61 - DNA, 22 bp, synthetic construct
  misc_feature 1..22: note = Exemplary cDNA sequence
  source 1..22: mol_type = other DNA
ctcttccgatcttggtaggctg

SEQ ID NO: 62 - DNA, 22 bp, synthetic construct
  misc_feature 1..22: note = Exemplary cDNA sequence
  source 1..22: mol_type = other DNA
cagcctaccaagatcggaagag

SEQ ID NO: 63 - DNA, 47 bp, synthetic construct
  misc_feature 1..47: note = Exemplary TAP backbone
  source 1..47: mol_type = other DNA
tatatatttgtggtatcaacgcagagtacacgacgctcttccgatct

SEQ ID NO: 64 - DNA, 35 bp, synthetic construct
  misc_feature 1..35: note = Exemplary cDNA sequence
  source 1..35: mol_type = other DNA
tctcatgtgctgcgagaaggctagaaccatccgac